SEQUENCE LISTING (196 sequences)

SEQ ID NO: 1 - DNA, 1602 bp, Solanum lycopersicum
  source 1..1602: mol_type = other DNA
atggggaattgctgtgggacacctggtaattcttctgagaataagaagaagaagaacaaaccaaacccttttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcaaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcagatgtgtcatatgcatggagtaatgcatcgtgatctcaaacctgagaactttctgtttggtaacaagaaagaaactgctcctttgaaggctattgactttggattatcagtgttctttaaacccggggaacgctttaatgagattgtgggaagtccttattacatggctccagaggtcctaaaacgcaattatggcccggaggttgatgtctggagtgctggagttatcctttatattcttctttgtggtgttccacctttctgggcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttgagcacacctggttacaaaatataaagaaagcaccaaatgtctcattgggtgagactgtgaaagcaaggcttaaacaattttcagtaatgaacaagctcaagaaaagagctctaacgattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgcggatgttgatggagatggaagcttaaattatggggagtttgttgctgtatctgttcatctcagaaaaatggcaaatgatgagcacctgcacaaagcattttcagtttttgacagagatcagagtggttacatagaaatcgaggagctgcgcagtgccttgagtgatgaggatggtggcaacagtgaggaagtcatcaatgccattatgcatgatgttgacactgacaaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 2 - protein, 533 aa, Solanum lycopersicum
MGNCCGTPGNSSENKKKKNKPNPFALDYGATQASGGDGNKLVVLKDPTGHNIQEKYDLGCELGRGEFGVTYLCTDVDTGDKYACKSISKKKLRTAVDIDDVRREVEIMKHLPKHPNIVTLKDTYEDDNAVHIVMELCEGGELFDRIVARGHYTERAAAVIMKTIVEVVQMCHMHGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVVDFKRDPWPKVSDNAKDLVKKMLDPDPTRRLTAQQVLEHTWLQNIKKAPNVSLGETVKARLKQFSVMNKLKKRALTIMAEFLSAEEVAGMKDAFDMMDTGKKGKINLGELKNGLQKLGHQIPDVDLQILMEAADVDGDGSLNYGEFVAVSVHLRKMANDEHLHKAFSVFDRDQSGYIEIEELRSALSDEDGGNSEEVINAIMHDVDTDKDGRISYEEFAAMMKAGTDWRKASRQYSRERFNSLSLKLMRDGSIQVGKEEGR

SEQ ID NO: 3 - DNA, 4914 bp, Solanum lycopersicum
  source 1..4914: mol_type = genomic DNA
atggggaattgctgtgggacacctggtaattcttctgagaataagaagaagaagaacaaaccaaacccttttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcaaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcaggtacagttttactaattaatgcttgtctttgcttttcatctataattgttacttgtttgtttatttcatgtaatttaggggttgctaatttaggattatgttaatgaagtttgaattgacaatgccaactgaaagtgtcaatttagactagaaaggaatgttgagattgaagtttcatgtttgtaagcagaatgaatgttcttgtcgaagaatctcttttaagaatatctttcccacttcttcagtaaattttcctactttcttatgtcgataactgccactccttttctgagtttgcctgcactctcttggtagataggacactccctttcttagctcaggatctactttgcttgtgtaaatgagtgcttaatgtttttatttgattcattttacacgttgagatcctatagcatttaattagactagattttatcttccaatatcactaaacctagatgtcaaatatagtaaaaagttatgttgctcgaactcttcaaagatatcgacatgtgtgtgtcagatcctccaaaagtagtgcatttttggaggatccgacgggggtggggcaacaattttggagagttcgagcaacttaggtaaaaagtgtattcatgctctaaatgttgttaggttttctatctttcttatcttgtcttgtgaattttgctttcttggcaactgtttcattgtcctatatttttctcatgcaaatacctttaaaattatatactttcgtgccaaccatttaaatgaaacttaaggaagctaatcacacaggatttcaccatccaaatgtcgaaaatgaatcgcaatttgtgatagttttagacagagagcttatttttatagatgcaattagaataatagaaatgtaaaataactatcagtaaccagtaagatgccggtctttatggtctattttcagagggggagatctattgtagcttgctttcagcatcagttagtatagcataggactgttgctaacagggttcaattttaaactgtatttggtgaacataatgacatgagagctcaacttacataactacagattagagagagacattcattttatggaatggatttaagaagttcatgtttctacatttagttgttataagaacagggtggtaactggtaaccgacgcaaacatgatggctaattatcccaagtttatacaatttaatctatgtggttgctcatacatctttggtcaatgttcactttacagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaactgctcctttgaaggctattgactttggattatcagtgttctttaaacccggtaatgcaataagtaatgatagtgtgttgtaactttttttcatgctaaaaaagattaatgttggaatcatcttttaatatgtttaacaggggaacgctttaatgagattgtgggaagtccttattacatggctccagaggtcctaaaacgcaattatggcccggaggttgatgtctggagtgctggagttatcctttatattcttctttgtggtgttccacctttctgggcaggtccgtttcttttatttgccttctttaggtaacaagtaatacctcggttcaccgacaaataacaaagtaaatcaaccttggtgcatactattcatttcttagaacttggtaatttattaatgagtgtcaaaatgaaatggaatttgaaacatattgatgcgttgttaacatcgtcgatgactaggtgagggtggctgcaagagtgaatcccacctcttcctggaccaattcaaaatcaaagttcctttttttccttttggcctgtgatagtcagtactttgtgcttccattaatttactcacagctttctcagtaattgcctaaaattctactgaatatgtgcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttggtaatgtttgttaatccttgagatgctatactttcatttgcttgctgtttgttctctcttagtttcataagttcaattttgtgacttttttggggctttcgtgcatatacatgaatgcagagcacacctggttacaaaatataaagaaagcaccaaatgtctcattgggtgagactgtgaaagcaaggcttaaacaattttcagtaatgaacaagctcaagaaaagagctctaacggtaaaatattcccttttcatttgcagttacatatcattagcgttctgcaccttctcagaaggatatcttcatatgcctaattcttaaagcccaaaataaggacaaaagattttctgccttctcacaaaagtttcgggtgttatattctttcttcattgcttgcacagattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgtaagcattctcatgctcttcatttcacttgattggattttctttcaactttacgaagactaaatatatatatttataagtcaagcgtctaatattattataattgttcccttttttatcttctgcttcacttttataggcggatgttgatggagatggaagcttaaattatggggagtttgttgctgtatctgttcatctcagaaaaatggcaaatgatgagcacctgcacaaagcattttcagtttttgacagagatcagagtggttacatagaaatcgaggagctgcgcagtgccttgagtgatgaggatggtggcaacagtgaggaagtcatcaatgccattatgcatgatgttgacactgacaaggttagaattttttttgtgtccattcatgtatatctactggatttcatactagtgcatatacttcttttataaatgtctctttaattgcttgtggaaccataaataccccccttatcttgttccgcagctccaactacacacgaaactatgttgaagacgtattacccccttgtacttgtaaaatgtgtatcttccgtcatcctgagaggcatatatgatgtaaccaaatgtgaacatttaagtatatgcatgtgctgcctcatgtcatttacccctttttcccttttcacttctcttccatctccgcaacatgttaatgtcctctagcaaatctgaaaaaagatggagtgagcttctcaacggaaaaaatcggattcaccctagttttagttttctggcaacataaacagctcaagtcgatacaaaaaaacaaatgttcctatcaaaaaactcaaattagtagaaaggaaaagaagggtcataggtaagggaagaacaaaaaggaatagaaagacccttcggggtggcccagttatttgggcttggggcttggggcttgggacttccatgttggaggtctcaagttcaaaaccccttgccaatgaaagcaacgggttttccttctacgttgactgtcgagctcgtcgcaccgggcttacctagtgcggtttacctctcctttgcgtgctataggaataggggttttaccctgtgtgcactcaaagggtagcgactgcggatttcccaaaagaaaaaggaagagaaaggtaaagaaaacattttaaaaaagagtaggatggttataatagaagaaatgctacaatattttttcacctcaggcgctttaagagagtaaatactctacgttacatggcgtcccaatgttgtcgtagttacattttggacaagaacagggagtaataggcctcccgcaaagttaaaatgtagtggggaatgggggaataattgaggaaggtatttatatattttctcctttctaaatcatattttcttttccattcactgctcttctgttcaaccttatcattcatttggactaaatgttgaattgattttgagcttttagtttgttaccacagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 4 - DNA, 19 bp, Solanum lycopersicum
  source 1..19: mol_type = other DNA
ttggttgtgagctaggaag

SEQ ID NO: 5 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 6 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 7 - DNA, 20 bp, synthetic construct
  source 1..20: mol_type = other DNA
atggggaattgctgtgggac

SEQ ID NO: 8 - DNA, 27 bp, synthetic construct
  source 1..27: mol_type = other DNA
ttcataataaccgcagctgctctctct

SEQ ID NO: 9 - DNA, 1602 bp, Solanum lycopersicum
  source 1..1602: mol_type = other DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcatgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcgtggaagtcgttcagatgtgtcacaggcaaggggtaatgcatcgtgatctcaaacctgagaattttctttttggtaacaagaaagaaacagctccactgaaggctattgactttgggttgtctgttttctttaaacctggggaacgctttaatgagatagtgggaagtccttattacatggctcctgaggtcctaaagcgcaattatggaccagaggtcgatgtctggagtgctggagttatactttacattcttctatgcggtgttccacctttctgggcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttgagcatccctggttacataatataaagaaagcaccaaacgtctcattgggtgagactgttaaagcaagactcaagcagttttcagtaatgaacaagctcaagaaaaaagctctgacggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 10 - protein, 533 aa, Solanum lycopersicum
MGNCCVKPGKSAEKKNKKNNSKPNPFSIDYGGTKHASGSGNKLVVLKEPTGQNIHDKYDLGHELGRGEFGVTYLCTDLEGGEKYACKSISKKKLRTAVDIDDVRREVEIMKHLPVHPNIVTLKDTYEDDNAVHIVMELCEGGELFDRIVARGHYTERAAAGILKTVVEVVQMCHRQGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVIDFKRDPWPKVSDNAKDLVKKMLDPDPTRRLTAHQVLEHPWLHNIKKAPNVSLGETVKARLKQFSVMNKLKKKALTVIAEFLSAEEVAGMKEAFEMMDTGKKGKINLNELKDGLQKLGHQIPDADLHILMEAADVDGDGSLNYPEFVAVSIHLRKMANDEHLHKAFSFFDKNQSGFIEIEELRSALRDEDDSNSEEVTNAIMHDVDTDKDGRISYEEFAAMMKAGTDWRKASRQYSRERFNSLSLKLMREGSLQVENKV

SEQ ID NO: 11 - DNA, 5526 bp, Solanum lycopersicum
  source 1..5526: mol_type = genomic DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcatgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcgtggaagtcgttcaggtatagtttttatcaacttcttagttctcagttacctactgcttttattaccttttgcttaggttatcttattatatccattattttcagcatagcttcttcattactgtatttccttttcatattgtttttatatatgtcttacttaggctgagggtccattggaaatagcctctctaccttcacaaggtaggagtaaggctgcgtacacactacccttcccacaccccacttgtgggactatactaagtatgttgttgttttagttctcaattgatgctttttggtttttatgtagtaacttgtggtgtttcttttactgagttcttcaaagcttttgtatgggttttatatcatgacggatgtatcctgtattgttgtttcgggtttttattaaagaagtctggcaatttcaagttaaaaagaaaatcatatcttaaaagagaagcctaatcttgagcaaatcttgagcagatagcaatttcagggtgaaatgtgaatattcttggtgaagttgcatatgaactggtaaccattattcaattcatttctagtgttgatctttgcaatcaatggtttcattctagtgtacgttcatgctgtagcatttaccttgatatcctcatggaatttcctttactgccaaaattataatgagaagtggttatcttttttaataatggtaaagtatgagaagaggttttcttatgttctcagtctttgctagttctatttctttactcgttttacttaacttttttagtttcttgtgaactgtagtattgctctatatcttctcactcaaattcatgttctaaatcttaaccattctagtgatataaatgaagttaattggtgatataggatatccccctccaaaatctaacaatctttcaactcatcatagtcttgggcagatagctagttagaaccgtaacaaatatttaggaaagtagatcattcgaaatagttatccttgtgaaatattattctttatcttcattattgaccattggttttcaaactctagtactcttaagtggaagactcacttcttgtttctgtggagtattagctatagcggcctacaacagaatttgattgttgcactcaaagttaaacagagttctacagtttagggaaatgataatggaatttaaatttagtttgtatactgaagtaaatatggttgacactatcagaaacaagtgctttttacttcaaatattgatctttatcctcattgcgaaccattgatgtccaaactgttttagatggaagagcaatttgttgttcatttgagtggaatagtcaccaacaacagctaattaactacgtatagcttagagagatgcgaaccaatggtaaatttaagttgtctcctgaagtagttactgattgttcatgataaaggtcattaaagttgaattctctatttagtcagtgtccttttctgcatcgactatatagtggcctgaatgacattacaaaatatttatgagttggtaggaatctttttttcatatgtgaatcatgaatgtgttcacaaatgatcaatcagagagtgattattgtattatgtggtgttgataacttttgaacttttcttcatttggtttgtgtttaactgttatctgaaaggcttggttggtggtactaaagctattggattagcagaaaactttatacatgtttatggtcatgtttcgctctccagtccccgtttttatttgtcaaaggtcagttcatatatttccaatggttttgcagatgtgtcacaggcaaggggtaatgcatcgtgatctcaaacctgagaattttctttttggtaacaagaaagaaacagctccactgaaggctattgactttgggttgtctgttttctttaaacctggtaatggaattggaaaaaaaacagtgagtttaagacttttatcatgctaaacaaaggtttaacatgttgattattttaacaggggaacgctttaatgagatagtgggaagtccttattacatggctcctgaggtcctaaagcgcaattatggaccagaggtcgatgtctggagtgctggagttatactttacattcttctatgcggtgttccacctttctgggcaggtctgcttattttatttcttcgttttttataaccatggaacgaacctcaatgcagatattttcatgtgcaatttttggaacttgataattgattaactaagattgtgaatatgaagtagaacttgattcatgttagaatatgttgttgaaatattagatgatgaggcaagtgaggtgaaagactgtccctgctttaatcaatgtagatcatgttcaattattttggtttctcttaatggttcttatgggttggactttgctaaataacttgcataaatatgttaacatattcttgatacttcccttaagttcctctgaatatttgcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttggtaatgtttgtggaatattgagatattgtactttattcattcgttgttttactctctctctctctctctctctctctctatctatctctgataagtatttttttccggggctttgactcttgtggtgcagagcatccctggttacataatataaagaaagcaccaaacgtctcattgggtgagactgttaaagcaagactcaagcagttttcagtaatgaacaagctcaagaaaaaagctctgacggtaagcacacacttagtctgcaatcatatttataagctctggattcttcactgtccactaaagatgaattcatatgcttttcctcatagatcgaaatgaaggagagaaggttgtgtcttaattatttactgcactgccttgtgctatttaataaaaacttaattctcatcgtaaaaaatgagaggcggttctcaaaattttgatcaagtttcgtttttttatcttgatatatgtactttcttgtattccaaacaggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggtaagcggtctcatgccctgcttcttggctcgatcagatttcttatagctctaccgggggtaacataatatttgaaagttagctgctaaacttttctgcctcaaagagaaaacaggtgcctgagatgatatggccatgtcttacgtagaactgtaaattacatcaatggacattattttaaaatgaccgaaaactacatggaatccatgtgaacttagtaaagaaaatattcaatggaagtaaaggataccaagtagttggattaaggctagattgttggtggtacactgacattagtgtctcaatgtttactaagagtctcagtttgattagagatttgcacgtagatgcagggataaccgtgagattaagaaaattttaagaaattcacggagatgaaaaagatctgtaatactcatatagcttagggccaagcccaactagtttgggatcaaatcgtagttgttgttgataagaatttagcattagacttttgtctgtcaccctactgtcaaaaaattgattgattacaaaatatattttgtcaatataagttgtaacatttttctaccatttcaatgtagaaaaaaggaaaatctgtagcaaaattagcttgacatttcgcttgtcttagcatccctggttcaatgcttattattatccagaaaggaaaaaaaaaacatttctcttgatccttttgcatcttcgacctgaaggatgcttatcttttttattattttatttccttgaacttttgttttatgtttttcttattctgtatcggttttacaggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggtcagtatgtccatttatgtgcacgtacaccatttagtaacttttcgttctttggttcatacttcatagtgcaaatattatgcattgttgctaatgtttacatcttaatgagttctcattttgaagttctcttctgaaccctcgtttgagtttagagctcaaatactccacctagttgtgacttattgtatctctaaagtccgtcatattatataaacgtattatactttgatggcatgcacactttctagcacatatgtgttgctaacgaaactctagtgctgtgacaaatgaaatacatgtatacattgatggcatctgttcttcccatcacatatgtactaccggtcattactctctagtgctgtgacattatgctctccctgttcgcttcatgagttacacggttggttaacctgctgctcgttacatgtctgctacagaattcaagttttgttagctgttttccatctcatctctaacaacatccccactcatttgtattagcttcttatctggagttttgtgagctcagtcttgtatcgtcttgtgtcaaaacgtgtgcaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 12 - DNA, 19 bp, Solanum lycopersicum
  source 1..19: mol_type = other DNA
tggggcatgagctcggaag

SEQ ID NO: 13 - RNA, 76 nt, synthetic construct; identical to SEQ ID NO: 5
  source 1..76: mol_type = other RNA

SEQ ID NO: 14 - DNA, 76 bp, synthetic construct; identical to SEQ ID NO: 6
  source 1..76: mol_type = other DNA

SEQ ID NO: 15 - DNA, 22 bp, synthetic construct
  source 1..22: mol_type = other DNA
agaacaacagcaaacctaaccc

SEQ ID NO: 16 - DNA, 20 bp, synthetic construct
  source 1..20: mol_type = other DNA
tccagcagctgctctctctg

SEQ ID NO: 17 - DNA, 2418 bp, Solanum lycopersicum
  source 1..2418: mol_type = other DNA
atggctcaacgtgttctaactcgtgttcacagtcttcgtgaacgtcttgatgctactttggatgctcatcgcaatgaaattttgctctttctttcaaggatcgaaagccacgggaaagggatcttgaaacctcaccagctactggctgagtttgaatcaattcagaaagaagacaaagacaaactgaatgatcatgcctttgaagaagtcctgaaatccactcaggaagcaattgttttgcccccatgggttgcacttgctattcgtttgaggcccggtgtgtgggaatatgtccgtgtgaatgttaatgctcttagtgttgaggagctgactgtgcctgagtttttgcaattcaaggaagaacttgttaacggaacttccagtgataactttgttcttgaattggattttgagcccttcactgcatcatttccaaaaccaaccctcacgaaatcaattggaaatggagttgaattcctcaacaggcacctctctgctaaaatgttccatgacaaggaaagcatgacccctcttctcgagtttcttcgagttcaccactacaatggaaagtcaatgatgctgaatgatagaattcagaatttgtatactctccaaaaagtcctgaggaaggccgaggaatacctcaccaccctttcgccagaaacttcatactcctcatttgagcacaagttccaagaaattggcttggagagaggttggggtgacaccgcagagcgtgttctagagatgatctgcatgctcctggatctccttgaggctcctgactcatgtactcttgagaagttccttagtagaattcctatggttttcaatgtagttatactttcacctcatggatatttcgcccaggaaaatgtcttgggttaccccgacactggtggtcaggttgtctatattttggatcaagttcctgccttggagcgtgagatgctcaagcgcataaaggagcaaggacttgatatcaaaccgcgtattcttattgttactcggcttctccctgatgcagttggtaccacttgtggtcagcgactcgagaaggtatttggaactgagcattcacatattcttagggtcccctttaggactgaaaagggcattgttcgcaaatggatctctcgttttgaagtctggccatacatggagactttcattgaggatgtggggaaagaaataaccgcagaactgcaagctaagccagatcttattattggaaactatagtgagggaaaccttgcagcctccttgttggctcacaagttaggtgtaacacagtgcaccattgctcatgcattggagaaaaccaaatatcctgattctgacatttacttgaacaaatttgacgagaaataccacttctcagctcagttcacagctgatcttatagcaatgaatcatactgatttcattatcaccagcaccttccaggagatagcaggaagcaaggacactgttggacagtatgagagccacatggccttcacaatgcctggattgtatagagttgttcatggcattgatgtgttcgaccccaaattcaacattgtgtcaccaggagctgatgtgaatctctatttcccatactccgaaaaggaaaagagattgacaacttttcaccctgaaattgaagacttgctgtttagcgatgttgagaacgaagaacacctgtgtgtgttgaaggacaggaataagcccatcatattcaccatggcaagattggaccgagtgaagaacttaactggacttgtcgagtggtatgctaagaatccacgactaagggagttggttaaccttgtagtggttggtggagaccgaagaaaggaatccaaagacttggaagagcaggcagagatgaagaagatgtatgaacttataaagactcacaatttgaatggccagttccgatggatttcttcccagatgaaccgcgtgaggaatggggaactctacaggtacattgctgacacaaggggagctttcgtgcagcctgcattctacgaggctttcggtctgactgttgttgaggccatgagctgcggtttgcctacatttgcaactaatcaaggtggtccagctgagatcatcgttcatggaaagtctggtttccaaattgatccataccatggcgagcaggctgctgatctcctcgctgagttcttcgagaaatgtaaggtagacccttcacattgggaagccatttccaagggtggccttaagcgtatacaggagaagtacacatggcaaatctactccgaccggctgttgacactagctgctgtttacgggttctggaagcacgtttccaagcttgatcgtcttgaaattcgtcgttatcttgagatgttttacgctctcaaattccgcaagctggctgaacttgtcccattggctgttgagtaa

SEQ ID NO: 18 - protein, 805 aa, Solanum lycopersicum
MAQRVLTRVHSLRERLDATLDAHRNEILLFLSRIESHGKGILKPHQLLAEFESIQKEDKDKLNDHAFEEVLKSTQEAIVLPPWVALAIRLRPGVWEYVRVNVNALSVEELTVPEFLQFKEELVNGTSSDNFVLELDFEPFTASFPKPTLTKSIGNGVEFLNRHLSAKMFHDKESMTPLLEFLRVHHYNGKSMMLNDRIQNLYTLQKVLRKAEEYLTTLSPETSYSSFEHKFQEIGLERGWGDTAERVLEMICMLLDLLEAPDSCTLEKFLSRIPMVFNVVILSPHGYFAQENVLGYPDTGGQVVYILDQVPALEREMLKRIKEQGLDIKPRILIVTRLLPDAVGTTCGQRLEKVFGTEHSHILRVPFRTEKGIVRKWISRFEVWPYMETFIEDVGKEITAELQAKPDLIIGNYSEGNLAASLLAHKLGVTQCTIAHALEKTKYPDSDIYLNKFDEKYHFSAQFTADLIAMNHTDFIITSTFQEIAGSKDTVGQYESHMAFTMPGLYRVVHGIDVFDPKFNIVSPGADVNLYFPYSEKEKRLTTFHPEIEDLLFSDVENEEHLCVLKDRNKPIIFTMARLDRVKNLTGLVEWYAKNPRLRELVNLVVVGGDRRKESKDLEEQAEMKKMYELIKTHNLNGQFRWISSQMNRVRNGELYRYIADTRGAFVQPAFYEAFGLTVVEAMSCGLPTFATNQGGPAEIIVHGKSGFQIDPYHGEQAADLLAEFFEKCKVDPSHWEAISKGGLKRIQEKYTWQIYSDRLLTLAAVYGFWKHVSKLDRLEIRRYLEMFYALKFRKLAELVPLAVE

SEQ ID NO: 19 - DNA, 3869 bp, Solanum lycopersicum
  source 1..3869: mol_type = genomic DNA
atggctcaacgtgttctaactcgtgttcacagtcttcgtgaacgtcttgatgctactttggatgctcatcgcaatgaaattttgctctttctttcaaggtatggtcttacagccatgttttcgctttttgtaattttcgtttggtatgtttcgcaagcctaaatagttttggggatacaaacttagtactgccttattcctttgtttggttattgatctcggaataagttatatcagaattagtaattagtacctcgataagttatctctgatggaataatagtactgttaactagtaatcctggaacaatacaatgaaaatgacaaaaatcccccagaggtcccttagatccttttttacttaataaggtggaaggtattctcgtaaacaaataactttcgttcttaaaaattatgcaatgaatgtttgtcctagtaaaacttgtcttcaaaccaaacgacccctaattgacggacaaatatatggttccatcaagtacatcatgagttattcttgctgtttttggctgtatcaggatcgaaagccacgggaaagggatcttgaaacctcaccagctactggctgagtttgaatcaattcagaaagaagacaaagacaaactgaatgatcatgcctttgaagaagtcctgaaatccactcaggtaacttggttttgttgttaggttgattatatttaattttcttactaagctaacatgtgtatctgtttgtacttgcatttgaaattttcttttgcgttgttcgtcatactaggaagcaattgttttgcccccatgggttgcacttgctattcgtttgaggcccggtgtgtgggaatatgtccgtgtgaatgttaatgctcttagtgttgaggagctgactgtgcctgagtttttgcaattcaaggaagaacttgttaacggaacgtaagttttacgtttgaatttgatgatgagttatctaatcaacatgttcttagaatctttttgattaatgttgtgattttccctgatgcagttccagtgataactttgttcttgaattggattttgagcccttcactgcatcatttccaaaaccaaccctcacgaaatcaattggaaatggagttgaattcctcaacaggcacctctctgctaaaatgttccatgacaaggaaagcatgacccctcttctcgagtttcttcgagttcaccactacaatggaaaggtaaatttagtgttatggtggtttttctccgataaatttcgagtatggagttactgatttttgcatccaacaaatgattaacatgttcgaacaatcattttctgtgcagtcaatgatgctgaatgatagaattcagaatttgtatactctccaaaaagtcctgaggaaggccgaggaatacctcaccaccctttcgccagaaacttcatactcctcatttgagcacaagttccaagaaattggcttggagagaggttggggtgacaccgcagagcgtgttctagagatgatctgcatgctcctggatctccttgaggctcctgactcatgtactcttgagaagttccttagtagaattcctatggttttcaatgtagttatactttcacctcatggatatttcgcccaggaaaatgtcttgggttaccccgacactggtggtcaggtgcattgcttatttgtgatcactttgtcttactccttagaaacctattggggcaagtgctgagttcccgttcctttaatttgcaggttgtctatattttggatcaagttcctgccttggagcgtgagatgctcaagcgcataaaggagcaaggacttgatatcaaaccgcgtattcttattgttagtattctcagagattgtgcttatagattatgattatgcaggattttgatttgttctaatgcaacatctgatctttgtttaaattctcaggttactcggcttctccctgatgcagttggtaccacttgtggtcagcgactcgagaaggtatttggaactgagcattcacatattcttagggtcccctttaggactgaaaagggcattgttcgcaaatggatctctcgttttgaagtctggccatacatggagactttcattgaggtgaagcaacttttctgtattcatttttcagtcttctagttgatttttgcagcaattttctacttacactaaaactgtgactttaatacattaggatgtggggaaagaaataaccgcagaactgcaagctaagccagatcttattattggaaactatagtgagggaaaccttgcagcctccttgttggctcacaagttaggtgtaacacaggtctgtaacattggtcacatgtataagattgactttgcatttcctttcatttggaactcggagttattaagaattctcttttgcgttgatctgcagtgcaccattgctcatgcattggagaaaaccaaatatcctgattctgacatttacttgaacaaatttgacgagaaataccacttctcagctcagttcacagctgatcttatagcaatgaatcatactgatttcattatcaccagcaccttccaggagatagcaggaaggtatgacatcaattcgctctatgattaaattgttcttcttcctgtttcattgtgtttgatcctaaaacatttttcaattttctttagcaaggacactgttggacagtatgagagccacatggccttcacaatgcctggattgtatagagttgttcatggcattgatgtgttcgaccccaaattcaacattgtgtcaccaggagctgatgtgaatctctatttcccatactccgaaaaggaaaagagattgacaacttttcaccctgaaattgaagacttgctgtttagcgatgttgagaacgaagaacacctgtatgtttctataatagtgcaacattgtgtttactgatataaacaagcttgtactgaaatagatttgtgttcctatatcaggtgtgtgttgaaggacaggaataagcccatcatattcaccatggcaagattggaccgagtgaagaacttaactggacttgtcgagtggtatgctaagaatccacgactaagggagttggttaaccttgtagtggttggtggagaccgaagaaaggaatccaaagacttggaagagcaggcagagatgaagaagatgtatgaacttataaagactcacaatttgaatggccagttccgatggatttcttcccagatgaaccgcgtgaggaatggggaactctacaggtacattgctgacacaaggggagctttcgtgcagcctgcattctacgaggctttcggtctgactgttgttgaggccatgagctgcggtttgcctacatttgcaactaatcaaggtggtccagctgagatcatcgttcatggaaagtctggtttccaaattgatccataccatggcgagcaggctgctgatctcctcgctgagttcttcgagaaatgtaaggtagacccttcacattgggaagccatttccaagggtggccttaagcgtatacaggagaagtaagctgctcatcttttcgtcctatcacgtgatcactattgagtgcattaattcaaagtgattctaatctcctgttgctgtaggtacacatggcaaatctactccgaccggctgttgacactagctgctgtttacgggttctggaagcacgtttccaagcttgatcgtcttgaaattcgtcgttatcttgagatgttttacgctctcaaattccgcaagctggtatgtctctctgctttctgcacttgtccaatagtctaaagcaactgttaatggagcttctgtattttgttttgattcaggctgaacttgtcccattggctgttgagtaa

SEQ ID NO: 20 - DNA, 21 bp, synthetic construct
  source 1..21: mol_type = other DNA
atggcatcctcatattgagat

SEQ ID NO: 21 - DNA, 20 bp, synthetic construct
  source 1..20: mol_type = other DNA
tattcctcggccttcctcag

SEQ ID NO: 22 - DNA, 12 bp, synthetic construct
  source 1..12: mol_type = other DNA
ttgttgttgttg

SEQ ID NO: 23 - DNA, 4897 bp, Solanum lycopersicum
  source 1..4897: mol_type = genomic DNA
atggggaattgctgtgggacacctggtaattcttctgagaataagaagaagaacaaaccaaacccgtttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcgaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttgttatgaagactatagtagaagttgttcaggtacagttttactaattgatgcttgtctttgcttttcatctataattgttacttgtttgtttatttcatgtaatttaggggttgctaatttaggattatgttaatgaagtttgaattgacaatgccaactgaaagtatcaatttagactagaaaggaatgttgagattgaagtttcatgtttgtaagcagaatgaatgttcttgtcgaagaatctcttttaagaatatctttcccacttcttcagtaaattttcctactttcttatgtcgataactgccactccttttctgagtttgcctgcactctcttggtagataggacactccctttcttagctcaggatttactttgcttgtgtaaatgagtgcttaatgtttttatttgattcattttacacgttgagatcctatagcatttaattagactagattttatcttccaatatcactaaacctagatgtcaaatatagtaaaaagctatgttgctcgaactcttcaaagatatcgacatgtgtgtgtcagatcctccaaaagtagtgcatttttggaggatccgacgggggtggggcaacaattttggagagttcgagcaacttaggtaaaaagtgtattcatgctctaaatgttgttaggttttctatctttcttatcttgtcttgtgaattttgctttcttggcaactgtttcattgtcctatatttttctcatgcaaatacctttaaaattatatactttcgtgccaaccatttaaatgaaacttaaggaagctaatcacacaggatttcaccatccaaatgtcgaaaatgaatcgcaatttgtgatagttttagacagagagcttatttttatagatgcaattagaataatagaaatgtaaaataactatcagtaaccagtaagatgccggtctttatggtctattttcagagggggagatctattgtagcttgctttcagcatcagttagtatagcataggactgttgctaacagggttcaattttaaactgtatttggtgaacataatgacatgagagctcaacttacataactacagattagagagagacattcattttatggaatggatttaagaagttcatgtttctacatttagttgttataagaacagggtggtaactggtaaccgacgcaaacatgatggctaattatcccaagtttatacaatttaatctatgtggttgctcatacatctttggtcaatgttcactttacagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaattgctcctttgaaggctattgactttggattatcagtgttctttaaacccggtaatgcaataagtaatgatagtgtgttgtaactttttttcatgctaaaaaagattaatgttggaatcatcttttaatatgtttaacaggggaacggtttaatgagattgtgggaagtccttattacatggctccagaggtcctaaaacgcaattatggcccggaggttgatgtctggagtgctggagttatcctttatattcttctttgtggtgttccacctttctgggcaggtccgtttcttttatttgccttctttaggtaacaagtaatacctcggttcaccgacaaataacaaattaaatcaaccttggtgcatactattcatttcttagaacttggtaatttattaatgagtgtcaaaatgaaatggaatttgaaacatattgatgcgttgttaacatcgtcgatgactaggtgagggtggctgcaagagtgaatcccacctcttcctggaccaattcaaaatcaaagttcctttttttccttttggcctgtgatagtcagtactttgtgcttccattaatttactcacagctttctcagtaattgcctaaaattctactgaatatgtgcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttggtaatgtttgttaatccttgagatgctatactttcatttgcttgctgtttgttctctcttagtttcataagttcaattttgtgacttttttggggctttcgtgcatatacatgaatgcagagcacacctggttacaaaatataaagaaagcaccaaatgtctcattgggtgagactgtgaaagcaaggcttaaacaattttcagtaatgaacaagctcaagaaaagagctctaacggtaaaatattcccttttcatttgcagttacatatcattagcgttctgcaccttctcagaaggatatcttcatatgcctaattcttaaagcccaaaataaggacaaaagattttctgccttctcacaaaagtttcgggtgttatattctttcttcattgcttgcacagattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgtaagcattctcatgctcttcatttcacttgattggattttctttcaactttacgaagactaaatatatatatttataagtcaagcgtctaatattattataattgttccctttttgatcttctgcttcacttttataggcggatgttgatggagatggaagcttaaattatggggagtttgttgctgtatctgttcatctcagaaaaatggcaaatgatgagcacctgcacaaagcattttcagttttcgacagagatcagagtggttacatagaaatcgaggagctgcgcagtgccttgagtgatgaggatggtggcaacagtgaggaagtcatcaatgccattatgcatgatgttgacactgacaaggttagaattttttttgtgtccattcatgtatatctactggatttcatactagtgcatatacttctcttataaatgtctctttaattgcttgtggaaccataaataccccccttatcttgttccgcagttccaactacacacgaaactatgttgaagacgtattacccccttgtacttgtaaaatgtgtatcttccgtcatcctgagaggcatatatgatgtaaccaaatgtgaacatttaagtatatgcatgtgctgcctcatgtcatttacccctttttcccttttcacttctcttccatctccgcaacatgttaatgtcctctagcaaatctgaaaaaagatggagtgagcttctcaacggaaaaaatcggattcaccctagttttagttttctggcaacataaacagctcaagtcgatacaaaaaaacaaatgttcctatcaaaaaactcaaattagtagaaaggaaaagaagggtcataggtaagggaagaacaaaaaggaatagaaagacccttcggggtggcccagttatttgggcttggggcttccatgttggaggtctcaagttcaaaaccccttgccaatgaaagcaacgggttttccttctacgttgactgtcgagctcgtcgcaccgggcttacctagtgcggtttacctctcctttgcgtgctataggaataggggttttaccctgtgtgcactcaaagggtagcgactgcggatttcccaaaagaaaaaggaagagaaaggtaaagaaaacattttaaaaaagagtaggatggttataatagaagaaatgctataatattttttcacctcaggcgctttaagagagtaaatactctacgttacatggcgtcccaatgttgtcgtagttacattttggacaagaacagggagtaataggcctcccgcaaagttaaaatgtagtagggaatgggggaataattgaggaaggtatttatatattttctcctttctaaatcatattttcttttccattcactgctcttctgttcaaccttatcattcatttggactaaatgttgaattgattttgagcttttagtttgttaccacagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 24 - DNA, 4914 bp, Solanum lycopersicum
  source 1..4914: mol_type = genomic DNA
atggggaattgctgtgggacacctggtaattcttctgagaataagaagaagaagaacaaaccaaacccttttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcaaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcaggtacagttttactaattaatgcttgtctttgcttttcatctataattgttacttgtttgtttatttcatgtaatttaggggttgctaatttaggattatgttaatgaagtttgaattgacaatgccaactgaaagtgtcaatttagactagaaaggaatgttgagattgaagtttcatgtttgtaagcagaatgaatgttcttgtcgaagaatctcttttaagaatatctttcccacttcttcagtaaattttcctactttcttatgtcgataactgccactccttttctgagtttgcctgcactctcttggtagataggacactccctttcttagctcaggatctactttgcttgtgtaaatgagtgcttaatgtttttatttgattcattttacacgttgagatcctatagcatttaattagactagattttatcttccaatatcactaaacctagatgtcaaatatagtaaaaagttatgttgctcgaactcttcaaagatatcgacatgtgtgtgtcagatcctccaaaagtagtgcatttttggaggatccgacgggggtggggcaacaattttggagagttcgagcaacttaggtaaaaagtgtattcatgctctaaatgttgttaggttttctatctttcttatcttgtcttgtgaattttgctttcttggcaactgtttcattgtcctatatttttctcatgcaaatacctttaaaattatatactttcgtgccaaccatttaaatgaaacttaaggaagctaatcacacaggatttcaccatccaaatgtcgaaaatgaatcgcaatttgtgatagttttagacagagagcttatttttatagatgcaattagaataatagaaatgtaaaataactatcagtaaccagtaagttgccggtctttatggtctattttcagagggggagatctattgtagcttgctttcagcatcagttagtatagcataggactgttgctaacagggttcaattttaaactgtatttggtgaacataatgacatgagagctcaacttacacaactacagattagagagagacattcattttatggaatggatttaagaagttcatgtttctacatttagttgttataagaacagggtggtaactggtaaccgacgcaaacatgatggctaattatcccaagtttatacaatttaatctatgtggttgctcatacatctttggtcaatgttcactttacagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaactgctcctttgaaggctattgactttggattatcagtgttctttaaacccggtaatgcaataagtaatgatagtgtgttgtaactttttttcatgctaaaaaagattaatgttggaatcatcttttaatatgtttaacaggggaacgctttaatgagattgtgggaagtccttattacatggctccagaggtcctaaaacgcaattatggcccggaggttgatgtctggagtgctggagttatcctttatattcttctttgtggtgttccacctttctgggcaggtccgtttcttttatttgccttctttaggtaacaagtaatacctcggttcaccgacaaataacaaagtaaatcaaccttggtgcatactattcatttcttagaacttggtaatttattaatgagtgtcaaaatgaaatggaatttgaaacatattgatgcgttgttaacatcgtcgatgactaggtgagggtggctgcaagagtgaatcccacctcttcctggaccaattcaaaatcaaagttcctttttttccttttggcctgtgatagtcagtactttgtgcttccattaatttactcacagctttctcagtaattgcctaaaattctactgaatatgtgcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttggtaatgtttgttaatccttgagatgctatactttcatttgcttgctgtttgttctctcttagtttcataagttcaattttgtgacttttttggggctttcgtgcatatacatgaatgcagagcacacctggttacaaaatataaagaaagcaccaaatgtctcattgggtgagactgtgaaagcaaggcttaaacaattttcagtaatgaacaagctcaagaaaagagctctaacggtaaaatattcccttttcatttgcagttacatatcattagcgttctgcaccttctcagaaggatatcttcatatgcctaattcttaaagcccaaaataaggacaaaagattttctgccttctcacaaaagtttcgggtgttatattctttcttcattgcttgcacagattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgtaagcattctcatgctcttcatttcacttgattggattttctttcaactttacgaagactaaatatatatatttataagtcaagcgtctaatattattataattgttcccttttttatcttctgcttcacttttataggcggatgttgatggagatggaagcttaaattatggggagtttgttgctgtatctgttcatctcagaaaaatggcaaatgatgagcacctgcacaaagcattttcagtttttgacagagatcagagtggttacatagaaatcgaggagctgcgcagtgccttgagtgatgaggatggtggcaacagtgaggaagtcatcaatgccattatgcatgatgttgacactgacaaggttagaattttttttgtgtccattcatgtatatctactggatttcatactagtgcatatacttctcttataaatgtctctttaattgcttgtggaaccataaataccccccttatcttgttccgcagctccaactacacacgaaactatgttgaagacgtattacccccttgtacttgtaaaatgtgtatcttccgtcatcctgagaggcatatatgatgtaaccaaatgtgaacatttaagtatatgcatgtgctgcctcatgtcatttacccctttttcccttttcacttctcttccatctccgcaacatgttaatgtcctctagcaaatctgaaaaaagatggagtgagcttctcaacggaaaaaatcggattcaccctagttttagttttctggcaacataaacagctcaagtcgatacaaaaaaacaaatgttcctatcaaaaaattcaaattagtagaaaggaaaagaagggtcataggtaagggaagaacaaaaaggaatagaaagacccttcggggtggcccagttatttgggcttggggcttggggcttgggacttccatgttggaggtctcaagttcaaaaccccttgccaatgaaagcaacgggttttccttctacgttgactgtcgagctcgtcgcaccgggcttacctagtgcggtttacctctcctttgcgtgctataggaataggggttttaccctgtgtgcactcaaagggtagcgactgcggatttcccaaaagaaaaaggaagagaaaggtaaagaaaacattttaaaaaagagtaggatggttataatagaagaaatgctacaatattttttcacctcaggcgctttaagagagtaaatactctacgttacatggcgtcccaatgttgtcgtagttacattttggacaagaacagggagtaataggcctcccgcaaagttaaaatgtagtggggaatgggggaataattgaggaaggtatttatatattttctcctttctaaatcatattttcttttccattcactgctcttctgttcaaccttatcattcatttggactaaatgttgaattgattttgagcttttagtttgttaccacagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 25 - DNA, 4897 bp, Solanum lycopersicum
  source 1..4897: mol_type = genomic DNA
atggggaattgctgtgggacacctggtaattcttctgagaataagaagaagaacaaaccaaacccgtttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcgaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcaggtacagttttactaattgatgcttgtctttgcttttcatctataattgttacttgtttgtttatttcatgtaatttaggggttgctaatttaggattatgttaatgaagtttgaattgacaatgccaactgaaagtatcaatttagactagaaaggaatgttgagattgaagtttcatgtttgtaagcagaatgaatgttcttgtcgaagaatctcttttaagaatatctttcccacttcttcagtaaattttcctactttcttatgtcgataactgccactccttttctgagtttgcctgcactctcttggtagataggacactccctttcttagctcaggatttactttgcttgtgtaaatgagtgcttaatgtttttatttgattcattttacacgttgagatcctatagcatttaattagactagattttatcttccaatatcactaaacctagatgtcaaatatagtaaaaagctatgttgctcgaactcttcaaagatatcgacatgtgtgtgtcagatcctccaaaagtagtgcatttttggaggatccgacgggggtggggcaacaattttggagagttcgagcaacttaggtaaaaagtgtattcatgctctaaatgttgttaggttttctatctttcttatcttgtcttgtgaattttgctttcttggcaactgtttcattgtcctatatttttctcatgcaaatacctttaaaattatatactttcgtgccaaccatttaaatgaaacttaaggaagctaatcacacaggatttcaccatccaaatgtcgaaaatgaatcgcaatttgtgatagttttagacagagagcttatttttatagatgcaattagaataatagaaatgtaaaataactatcagtaaccagtaagatgccggtctttatggtctattttcagagggggagatctattgtagcttgctttcagcatcagttagtatagcataggactgttgctaacagggttcaattttaaactgtatttggtgaacataatgacatgagagctcaacttacataactacagattagagagagacattcattttatggaatggatttaagaagttcatgtttctacatttagttgttataagaacagggtggtaactggtaaccgacgcaaacatgatggctaattatcccaagtttatacaatttaatctatgtggttgctcatacatctttggtcaatgttcactttacagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaattgctcctttgaaggctattgactttggattatcagtgttctttaaacccggtaatgcaataagtaatgatagtgtgttgtaactttttttcatgctaaaaaagattaatgttggaatcatcttttaatatgtttaacaggggaacggtttaatgagattgtgggaagtccttattacatggctccagaggtcctaaaacgcaattatggcccggaggttgatgtctggagtgctggagttatcctttatattcttctttgtggtgttccacctttctgggcaggtccgtttcttttatttgccttctttaggtaacaagtaatacctcggttcaccgacaaataacaaattaaatcaaccttggtgcatactattcatttcttagaacttggtaatttattaatgagtgtcaaaatgaaatggaatttgaaacatattgatgcgttgttaacatcgtcgatgactaggtgagggtggctgcaagagtgaatcccacctcttcctggaccaattcaaaatcaaagttcctttttttccttttggcctgtgatagtcagtactttgtgcttccattaatttactcacagctttctcagtaattgcctaaaattctactgaatatgtgcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttggtaatgtttgttaatccttgagatgctatactttcatttgcttgctgtttgttctctcttagtttcataagttcaattttgtgacttttttggggctttcgtgcatatacatgaatgcagagcacacctggttacaaaatataaagaaagcaccaaatgtctcattgggtgagactgtgaaagcaaggcttaaacaattttcagtaatgaacaagctcaagaaaagagctctaacggtaaaatattcccttttcatttgcagttacatatcattagcgttctgcaccttctcagaaggatatcttcatatgcctaattcttaaagcccaaaataaggacaaaagattttctgccttctcacaaaagtttcgggtgttatattctttcttcattgcttgcacagattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgtaagcattctcatgctcttcatttcacttgattggattttctttcaactttacgaagactaaatatatatatttataagtcaagcgtctaatattattataattgttccctttttgatcttctgcttcacttttataggcggatgttgatggagatggaagcttaaattatggggagtttgttgctgtatctgttcatctcagaaaaatggcaaatgatgagcacctgcacaaagcattttcagttttcgacagagatcagagtggttacatagaaatcgaggagctgcgcagtgccttgagtgatgaggatggtggcaacagtgaggaagtcatcaatgccattatgcatgatgttgacactgacaaggttagaattttttttgtgtccattcatgtatatctactggatttcatactagtgcatatacttctcttataaatgtctctttaattgcttgtggaaccataaataccccccttatcttgttccgcagttccaactacacacgaaactatgttgaagacgtattacccccttgtacttgtaaaatgtgtatcttccgtcatcctgagaggcatatatgatgtaaccaaatgtgaacatttaagtatatgcatgtgctgcctcatgtcatttacccctttttcccttttcacttctcttccatctccgcaacatgttaatgtcctctagcaaatctgaaaaaagatggagtgagcttctcaacggaaaaaatcggattcaccctagttttagttttctggcaacataaacagctcaagtcgatacaaaaaaacaaatgttcctatcaaaaaactcaaattagtagaaaggaaaagaagggtcataggtaagggaagaacaaaaaggaatagaaagacccttcggggtggcccagttatttgggcttggggcttccatgttggaggtctcaagttcaaaaccccttgccaatgaaagcaacgggttttccttctacgttgactgtcgagctcgtcgcaccgggcttacctagtgcggtttacctctcctttgcgtgctataggaataggggttttaccctgtgtgcactcaaagggtagcgactgcggatttcccaaaagaaaaaggaagagaaaggtaaagaaaacattttaaaaaagagtaggatggttataatagaagaaatgctataatattttttcacctcaggcgctttaagagagtaaatactctacgttacatggcgtcccaatgttgtcgtagttacattttggacaagaacagggagtaataggcctcccgcaaagttaaaatgtagtagggaatgggggaataattgaggaaggtatttatatattttctcctttctaaatcatattttcttttccattcactgctcttctgttcaaccttatcattcatttggactaaatgttgaattgattttgagcttttagtttgttaccacagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 26 - DNA, 4913 bp, Solanum lycopersicum
  source 1..4913: mol_type = genomic DNA
atggggaattgctgtgggacacctggtaattcttctgagaataagaagaagaagaacaaaccaaacccttttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcaaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcaggtacagttttactaattaatgcttgtctttgcttttcatctataattgttacttgtttgtttatttcatgtaatttaggggttgctaatttaggattatgttaatgaagtttgaattgacaatgccaactgaaagtgtcaatttagactagaaaggaatgttgagattgaagtttcatgtttgtaagcagaatgaatgttcttgtcgaagaatctcttttaagaatatctttcccacttcttcagtaaattttcctactttcttatgtcgataactgccactccttttctgagtttgcctgcactctcttggtagataggacactccctttcttagctcaggatctactttgcttgtgtaaatgagtgcttaatgtttttatttgattcattttacacgttgagatcctatagcatttaattagactagattttatcttccaatatcactaaacctagatgtcaaatatagtaaaaagttatgttgctcgaactcttcaaagatatcgacatgtgtgtgtcagatcctccaaaagtagtgcatttttggaggatccgacgggggtggggcaacaattttggagagttcgagcaacttaggtaaaaagtgtattcatgctctaaatgttgttaggttttctatctttcttatcttgtcttgtgaattttgctttcttggcaactgtttcattgtcctatatttttctcatgcaaatacctttaaaattatatactttcgtgccaaccatttaaatgaaacttaaggaagctaatcacacaggatttcaccatccaaatgtcgaaaatgaatcgcaatttgtgatagttttagacagagagcttatttttatagatgcaattagaataatagaaatgtaaaataactatcagtaaccagtaagttgccggtctttatggtctattttcagagggggagatctattgtagcttgctttcagcatcagttagtatagcataggactgttgctaacagggttcaattttaaactgtatttggtgaacataatgacatgagagctcaacttacacaactacagattagagagagacattcattttatggaatggatttaagaagttcatgtttctacatttagttgttataagaacagggtggtaactggtaaccgacgcaaacatgatggctaattatcccaagtttatacaatttaatctatgtggttgctcatacatctttggtcaatgttcactttacagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaactgctcctttgaaggctattgactttggattatcagtgttctttaaacccggtaatgcaataagtaatgatagtgtgttgtaactttttttcatgctaaaaaagattaatgttggaatcatcttttaatatgtttaacaggggaacgctttaatgagattgtgggaagtccttattacatggctccagaggtcctaaaacgcaattatggcccggaggttgatgtctggagtgctggagttatcctttatattcttctttgtggtgttccacctttctgggcaggtccgtttcttttatttgccttctttaggtaacaagtaatacctcggttcaccgacaaataacaaagtaaatcaaccttggtgcatactattcatttcttagaacttggtaatttattaatgagtgtcaaaatgaaatggaatttgaaacatattgatgcgttgttaacatcgtcgatgactaggtgagggtggctgcaagagtgaatcccacctcttcctggaccaattcaaaatcaaagttcctttttttccttttggcctgtgatagtcagtacattgtgcttccattaatttactcacagctttctcagtaattgcctaaaattctactgaatatgtgcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttggtaatgtttgttaatccttgagatgctatactttcatttgcttgctgtttgttctctcttagtttcataagttcaattttgtgacttttttggggctttcgtgcatatacatgaatgcagagcacacctggttacaaaatataaagaaagcaccaaatgtctcattgggtgagactgtgaaagcaaggcttaaacaattttcagtaatgaacaagctcaagaaaagagctctaacggtaaaatattcccttttcatttgcagttacatatcattagcgttctgcaccttctcagaaggatatcttcatatgcctaattcttaaagcccaaaataaggacaaaagattttctgccttctcacaaaagtttcgggtgttatattctttcttcattgcttgcacagattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgtaagcattctcatgctcttcatttcacttgattggattttctttcaactttacgaagactaaatatatatatttataagtcaagcgtctaatattattataattgttcccttttttatcttctgcttcacttttataggcggatgttgatggagatggaagcttaaattatggggagtttgttgctgtatctgttcatctcagaaaaatggcaaatgatgagcacctgcacaaagcattttcagtttttgacagagatcagagtggttacatagaaatcgaggagctgcgcagtgccttgagtgatgaggatggtggcaacagtgaggaagtcatcaatgccattatgcatgatgttgacactgacaaggttagaattttttttgtgtccattcatgtatatctactggatttcatactagtgcatatacttctcttataaatgtctctttaattgcttgtggaaccataaataccccccttatcttgttccgcagctccaactacacacgaaactatgttgaagacgtattacccccttgtacttgtaaaatgtgtatcttccgtcatcctgagaggcatatatgatgtaaccaaatgtgaacatttaagtatatgcatgtgctgcctcatgtcatttacccctttttcccttttcacttctcttccatctccgcaacatgttaatgtcctctagcaaatctgaaaaaagatggagtgagcttctcaacggaaaaaatcggattcaccctagttttagttttctggcaacataaacagctcaagtcgatacaaaaaaacaaatgttcctatcaaaaaattcaaattagtagaaaggaaaagaagggtcataggtaagggaagaacaaaaaggaatagaaagacccttcggggtggcccagttatttgggcttggggcttggggcttgggacttccatgttggaggtctcaagttcaaaaccccttgccaatgaaagcaacgggttttccttctacgttgactgtcgagctcgtcgcaccgggcttacctagtgcggtttacctctcctttgcgtgctataggaataggggttttaccctgtgtacactcaaagggtagcgactgcggatttcccaaaagaaaaaggaagagaaaggtaaagaaaacattttaaaaaagagtaggatggttataatagaagaaatgctacaatattttttcacctcaggcgctttaagagagtaaatactctacgttacatggcgtcccaatgttgtcgtagttacattttggacaagaacagggagtaataggcctcccgcaaagttaaaatgtagtggggaatgggggaataattgaggaaggtatttatatattttctccttctaaatcatattttcttttccattcactgctcttctgttcaaccttatcattcatttggactaaatgttgaattgattttgagcttttagtttgttaccacagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 27 - DNA, 4914 bp, Solanum lycopersicum
  source 1..4914: mol_type = genomic DNA
atggggaattgctgtgggacacctggtaattcttctgagaataagaagaagaagaacaaaccaaacccttttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcaaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcaggtacagttttactaattaatgcttgtctttgcttttcatctataattgttacttgtttgtttatttcatgtaatttaggggttgctaatttaggattatgttaatgaagtttgaattgacaatgccaactgaaagtgtcaatttagactagaaaggaatgttgagattgaagtttcatgtttgtaagcagaatgaatgttcttgtcgaagaatctcttttaagaatatctttcccacttcttcagtaaattttcctactttcttatgtcgataactgccactccttttctgagtttgcctgcactctcttggtagataggacactccctttcttagctcaggatctactttgcttgtgtaaatgagtgcttaatgtttttatttgattcattttacacgttgagatcctatagcatttaattagactagattttatcttccaatatcactaaacctagatgtcaaatatagtaaaaagttatgttgctcgaactcttcaaagatatcgacatgtgtgtgtcagatcctccaaaagtagtgcatttttggaggatccgacgggggtggggcaacaattttggagagttcgagcaacttaggtaaaaagtgtattcatgctctaaatgttgttaggttttctatctttcttatcttgtcttgtgaattttgctttcttggcaactgtttcattgtcctatatttttctcatgcaaatacctttaaaattatatactttcgtgccaaccatttaaatgaaacttaaggaagctaatcacacaggatttcaccatccaaatgtcgaaaatgaatcgcaatttgtgatagttttagacagagagcttatttttatagatgcaattagaataatagaaatgtaaaataactatcagtaaccagtaagatgccggtctttatggtctattttcagagggggagatctattgtagcttgctttcagcatcagttagtatagcataggactgttgctaacagggttcaattttaaactgtatttggtgaacataatgacatgagagctcaacttacataactacagattagagagagacattcattttatggaatggatttaagaagttcatgtttctacatttagttgttataagaacagggtggtaactggtaaccgacgcaaacatgatggctaattatcccaagtttatacaatttaatctatgtggttgctcatacatctttggtcaatgttcactttacagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaactgctcctttgaaggctattgactttggattatcagtgttctttaaacccggtaatgcaataagtaatgatagtgtgttgtaactttttttcatgctaaaaaagattaatgttggaatcatcttttaatatgtttaacaggggaacgctttaatgagattgtgggaagtccttattacatggctccagaggtcctaaaacgcaattatggcccggaggttgatgtctggagtgctggagttatcctttatattcttctttgtggtgttccacctttctgggcaggtccgtttcttttatttgccttctttaggtaacaagtaatacctcggttcaccgacaaataacaaagtaaatcaaccttggtgcatactattcatttcttagaacttggtaatttattaatgagtgtcaaaatgaaatggaatttgaaacatattgatgcgttgttaacatcgtcgatgactaggtgagggtggctgcaagagtgaatcccacctcttcctggaccaattcaaaatcaaagttcctttttttccttttggcctgtgatagtcagtactttgtgcttccattaatttactcacagctttctcagtaattgcctaaaattctactgaatatgtgcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttggtaatgtttgttaatccttgagatgctatactttcatttgcttgctgtttgttctctcttagtttcataagttcaattttgtgacttttttggggctttcgtgcatatacatgaatgcagagcacacctggttacaaaatataaagaaagcaccaaatgtctcattgggtgagactgtgaaagcaaggcttaaacaattttcagtaatgaacaagctcaagaaaagagctctaacggtaaaatattcccttttcatttgcagttacatatcattagcgttctgcaccttctcagaaggatatcttcatatgcctaattcttaaagcccaaaataaggacaaaagattttctgccttctcacaaaagtttcgggtgttatattctttcttcattgcttgcacagattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgtaagcattctcatgctcttcatttcacttgattggattttctttcaactttacgaagactaaatatatatatttataagtcaagcgtctaatattattataattgttcccttttttatcttctgcttcacttttataggcggatgttgatggagatggaagcttaaattatggggagtttgttgctgtatctgttcatctcagaaaaatggcaaatgatgagcacctgcacaaagcattttcagtttttgacagagatcagagtggttacatagaaatcgaggagctgcgcagtgccttgagtgatgaggatggtggcaacagtgaggaagtcatcaatgccattatgcatgatgttgacactgacaaggttagaattttttttgtgtccattcatgtatatctactggatttcatactagtgcatatacttcttttataaatgtctctttaattgcttgtggaaccataaataccccccttatcttgttccgcagctccaactacacacgaaactatgttgaagacgtattacccccttgtacttgtaaaatgtgtatcttccgtcatcctgagaggcatatatgatgtaaccaaatgtgaacatttaagtatatgcatgtgctgcctcatgtcatttacccctttttcccttttcacttctcttccatctccacaacatgttaatgtcctctagcaaatctgaaaaaagatggagtgagcttctcaacggaaaaaatcggattcaccctagttttagttttctggcaacataaacagctcaagtcgatacaaaaaaacaaatgttcctatcaaaaaactcaaattagtagaaaggaaaagaagggtcataggtaagggaagaacaaaaaggaatagaaagacccttcggggtggcccagttatttgggcttggggcttggggcttgggacttccatgttggaggtctcaagttcaaaaccccttgccaatgaaagcaacgggttttccttctacgttgactgtcgagctcgtcgcaccgggcttacctagtgcggtttacctctcctttgcgtgctataggaataggggttttaccctgtgtgcactcaaagggtagcgactgcggatttcccaaaagaaaaaggaagagaaaggtaaagaaaacattttaaaaaagagtaggatggttataatagaagaaatgctacaatattttttcacctcaggcgctttaagagagtaaatactctacgttacatggcgtcccaatgttgtcgtagttacattttggacaagaacagggagtaataggcctcccgcaaagttaaaatgtagtggggaatgggggaataattgaggaaggtatttatatattttctcctttctaaatcatattttcttttccattcactgctcttctgttcaaccttatcattcatttggactaaatgttgaattgattttgagcttttagtttgttaccacagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 28 - DNA, 4897 bp, Solanum lycopersicum
  source 1..4897: mol_type = genomic DNA
atggggaattgctgtgggacacctggtaattcttctgagaataagaagaagaacaaaccaaacccgtttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcgaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcaggtacagttttactaattgatgcttgtctttgcttttcatctataattgttacttgtttgtttatttcatgtaatttaggggttgctaatttaggattatgttaatgaagtttgaattgacaatgccaactgaaagtatcaatttagactagaaaggaatgttgagattgaagtttcatgtttgtaagcagaatgaatgttcttgtcgaagaatctcttttaagaatatctttcccacttcttcagtaaattttcctactttcttatgtcgataactgccactccttttctgagtttgcctgcactctcttggtagataggacactccctttcttagctcaggatttactttgcttgtgtaaatgagtgcttaatgtttttatttgattcattttacacgttgagatcctatagcatttaattagactagattttatcttccaatatcactaaacctagatgtcaaatatagtaaaaagttatgttgctcgaactcttcaaagatatcgacatgtgtgtgtcagatcctccaaaagtagtgcatttttggaggatccgacgggggtggggcaacaattttggagagttcgagcaacttaggtaaaaagtgtattcatgctctaaatgttgttaggttttctatctttcttatcttgtcttgtgaattttgctttcttggcaactgtttcattgtcctatatttttctcatgcaaatacctttaaaattatatactttcgtgccaaccatttaaatgaaacttaaggaagctaatcacacaggatttcaccatccaaatgtcgaaaatgaatcgcaatttgtgatagttttagacagagagcttatttttatagatgcaattagaataatagaaatgtaaaataactatcagtaaccagtaagatgccggtctttatggtctattttcagagggggagatctattgtagcttgctttcagcattagttagtatagcataggactgttgctaacagggttcaattttaaactgtatttggtgaacataatgacatgagagctcaacttacataactacagattagagagagacattcattttatggaatggatttaagaagttcatgtttctacatttagttgttataagaacagggtggtaactggtaaccgacgcaaacatgatggctaattatcccaagtttatacaatttaatctatgtggttgctcatacatctttggtcaatgttcactttacagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaattgctcctttgaaggctattgactttggattatcagtgttctttaaacccggtaatgcaataagtaatgatagtgtgttgtaactttttttcatgctaaaaaagattaatgttggaatcatcttttaatatgtttaacaggggaacggtttaatgagattgtgggaagtccttattacatggctccagaggtcctaaaacgcaattatggcccggaggttgatgtctggagtgctggagttatcctttatattcttctttgtggtgttccacctttctgggcaggtccgtttcttttatttgccttctttaggtaacaagtaatacctcggttcaccgacaaataacaaattaaatcaaccttggtgcatactattcatttcttagaacttggtaatttattaatgagtgtcaaaatgaaatggaatttgaaacatattgatgcgttgttaacatcgtcgatgactaggtgagggtggctgcaagagtgaatcccacctcttcctggaccaattcaaaatcaaagttcctttttttccttttggcctgtgatagtcagtactttgtgcttccattaatttactcacagctttctcagtaattgcctaaaattctactgaatatgtgcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttggtaatgtttgttaatccttgagatgctatactttcatttgcttgctgtttgttctctcttagtttcataagttcaattttgtgactcttttggggctttcgtgcatatacatgaatgcagagcacacctggttacaaaatataaagaaagcaccaaatgtctcattgggtgagactgtgaaagcaaggcttaaacaattttcagtaatgaacaagctcaagaaaagagctctaacggtaaaatattcccttttcatttgcagttacatatcattagcgttctgcaccttctcagaaggatatcttcatatgcctaattcttaaagcccaaaataaggacaaaagattttctgccttctcacaaaagtttcgggtgttatattctttcttcattgcttgcacagattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgtaagcattctcatgctcttcatttcacttgattggattctctttcaactttacgaagactaaatatatatatttataagtcaagcgtctaatattattataattgttccctttttgatcttctgcttcacttttataggcggatgttgatggagatggaagcttaaattatggggagtttgttgctgtatctgttcatctcagaaaaatggcaaatgatgagcacctgcacaaagcattttcagttttcgacagagatcagagtggttacatagaaatcgaggagctgcgcagtgccttgagtgatgaggatggtggcaacagtgaggaagtcatcaatgccattatgcatgatgttgacactgacaaggttagaattttttttgtgtccattcatgtatatctactggatttcatactagtgcatatacttctcttataaatgtctctttaattgcttgtggaaccataaataccccccttatcttgttccgcagttccaactacacacgaaactatgttgaagacgtattacccccttgtacttgtaaaatgtgtatcttccgtcatcctgagaggcatatatgatgtaaccaaatgtgaacatttaagtatatgcatgtgctgcctcatgtcatttacccctttttcccttttcacttctcttccatctccgcaacatgttaatgtcctctagcaaatctgaaaaaagatggagtgagcttctcaacggaaaaaatcggattcaccctagttttagttttctggcaacataaacagctcaagtcgatacaaaaaaacaaatgttcctatcaaaaaactcaaattagtagaaaggaaaagaagggtcataggtaagggaagaacaaaaaggaatagaaagacccttcggggtggcccagttatttgggcttggggcttccatgttggaggtctcaagttcaaaaccccttgccaatgaaagcaacgggttttccttctacgttgactgtcgagctcgtcgcaccgggcttacctagtgcggtttacctctcctttgcgtgctataggaataggggttttaccctgtgtgcactcaaagggtagcgactgcggatttcccaaaagaaaaaggaagagaaaggtaaagaaaacattttaaaaaagagtaggatggttataatagaagaaatgctataatattttttcacctcaggcgctttaagagagtaaatactctacgttacatggcgtcccaatgttgtcgtagttacattttggacaagaacagggagtaataggcctcccgcaaagttaaaatgtagtagggaatgggggaataattgaggaaggtatttatatattttctcctttctaaatcatattttcttttccattcactgctcttctgttcaaccttatcattcatttggactaaatgttgaattgattttgagcttttagtttgttaccacagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 29 - DNA, 4897 bp, Solanum lycopersicum
  source 1..4897: mol_type = genomic DNA
atggggaattgctgtgggacacctggtaattcttctgagaataagaagaagaacaaaccaaacccgtttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcgaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcaggtacagttttactaattgatgcttgtctttgcttttcatctataattgttacttgtttgtttatttcatgtaatttaggggttgctaatttaggattatgttaatgaagtttgaattgacaatgccaactgaaagtatcaatttagactagaaaggaatgttgagattgaagtttcatgtttgtaagcagaatgaatgttcttgtcgaagaatctcttttaagaatatctttcccacttcttcagtaaattttcctactttcttatgtcgataactgccactccttttctgagtttgcctgcactctcttggtagataggacactccctttcttagctcaggatttactttgcttgtgtaaatgagtgcttaatgtttttatttgattcattttacacgttgagatcctatagcatttaattagactagattttatcttccaatatcactaaacctagatgtcaaatatagtaaaaagttatgttgctcgaactcttcaaagatatcgacatgtgtgtgtcagatcctccaaaagtagtgcatttttggaggatccgacgggggtggggcaacaattttggagagttcgagcaacttaggtaaaaagtgtattcatgctctaaatgttgttaggttttctatctttcttatcttgtcttgtgaattttgctttcttggcaactgtttcattgtcctatatttttctcatgcaaatacctttaaaattatatactttcgtgccaaccatttaaatgaaacttaaggaagctaatcacacaggatttcaccatccaaatgtcgaaaatgaatcgcaatttgtgatagttttagacagagagcttatttttatagatgcaattagaataatagaaatgtaaaataactatcagtaaccagtaagatgccggtctttatggtctattttcagagggggagatctattgtagcttgctttcagcatcagttagtatagcataggactgttgctaacagggttcaattttaaactgtatttggtgaacataatgacatgagagctcaacttacataactacagattagagagagacattcattttatggaatggatttaagaagttcatgtttctacatttagttgttataagaacagggtggtaactggtaaccgacgcaaacatgatggctaattatcccaagtttatacaatttaatctatgtggttgctcatacatctttggtcaatgttcactttacagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaattgctcctttgaaggctattgactttggattatcagtgttctttaaacccggtaatgcaataagtaatgatagtgtgttgtaactttttttcatgctaaaaaagattaatgttggaatcatcttttaatatgtttaacaggggaacggtttaatgagattgtgggaagtccttattacatggctccagaggtcctaaaacgcaattatggcccggaggttgatgtctggagtgctggagttatcctttatattcttctttgtggtgttccacctttctgggcaggtccgtttcttttatttgccttctttaggtaacaagtaatacctcggttcaccgacaaataacaaattaaatcaaccttggtgcatactattcatttcttagaacttggtaatttattaatgagtgtcaaaatgaaatggaatttgaaacatattgatgcgttgttaacatcgtcgatgactaggtgagggtggctgcaagagtgaatcccacctcttcctggaccaattcaaaatcaaagttcctttttttccttttggcctgtgatagtcagtactttgtgcttccattaatttactcacagctttctcagtaattgcctaaaattctactgaatatgtgcagagactgaacaaggagtagcccaagcaattattcggtctgtggttgatttcaagagagatccatggcctaaggtttctgataatgcaaaggatcttgtaaagaagatgcttgacccagatccaactcgacgactcacagctcagcaggttcttggtaatgtttgttaatccttgagatgctatactttcatttgcttgctgtttgttctctcttagtttcataagttcaattttgtgacttttttggggctttcgtgcatatacatgaatgcagagcacacctggttacaaaatataaagaaagcaccaaatgtctcattgggtgagactgtgaaagcaaggcttaaacaattttcagtaatgaacaagctcaagaaaagagctctaacggtaaaatattcccttttcatttgcagttacatatcattagcgttctgcaccttctcagaaggatatcttcatatgcctaattcttaaagcccaaaataaggacaaaagattttctgccttctcacaaaagtttcgggtgttatattctttcttcattgcttgcacagattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgtaagcattctcatgctcttcatttcacttgattggattttctttcaactttacgaagactaaatatatatatttataagtcaagcgtctaatattattataattgttccctttttgatcttctgcttcacttttataggcggatgttgatggagatggaagcttaaattatggggagtttgttgctgtatctgttcatctcagaaaaatggcaaatgatgagcacctgcacaaagcattttcagttttcgacagagatcagagtggttacatagaaatcgaggagctgcgcagtgccttgagtgatgaggatggtggcaacagtgaggaagtcatcaatgccattatgcatgatgttgacactgacaaggttagaattttttttgtgtccattcatgtatatctactggatttcatactagtgcatatacttctcttataaatgtctctttaattgcttgtggaaccataaataccccccttatcttgttccgcagttccaactacacacgaaactatgttgaagacgtattacccccttgtacttgtaaaatgtgtatcttccgtcatcctgagaggcatatatgatgtaaccaaatgtgaacatttaagtatatgcatgtgctgcctcatgtcatttacccctttttcccttttcacttctcttccatctccgcaacatgttaatgtcctctagcaaatctgaaaaaagatggagtgagcttctcaacggaaaaaatcggattcaccctagttttagttttctggcaacataaacagctcaagtcgatacaaaaaaacaaatgttcctatcaaaaaactcaaattagtagaaaggaaaagaagggtcataggtaagggaagaacaaaaaggaatagaaagacccttcggggtggcccagttatttgggcttggggcttccatgttggaggtctcaagttcaaaaccccttgccaatgaaagcaacgggttttccttctacgttgactgtcgagctcgtcgcaccgggcttacctagtgcggtttacctctcctttgcgtgctataggaataggggttttaccctgtgtgcactcaaagggtagcgactgcggatttcccaaaagaaaaaggaagagaaaggtaaagaaaacattttaaaaaagagtaggatggttataatagaagaaatgctataatattttttcacctcaggcgctttaagagagtaaatactctacgttacatggcgtcccaatgttgtcgtagttacattttggacaagaacagggagtaataggcctcccgcaaagttaaaatgtagtagggaatgggggaataattgaggaaggtatttatatattttctcctttctaaatcatattttcttttccattcactgctcttctgttcaaccttatcattcatttggactaaatgttgaattgattttgagcttttagtttgttaccacagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 30 - DNA, 4897 bp, Solanum lycopersicum
  source 1..4897: mol_type = genomic DNA
atggggaattgctgtgggacacctggtaattcttctgagaataagaagaagaacaaaccaaacccgtttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcgaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcaggtacagttttactaattgatgcttgtctttgcttttcatctataattgttacttgtttgtttatttcatgtaatttaggggttgctaatttaggattatgttaatgaagtttgaattgacaatgccaactgaaagtatcaatttagactagaaaggaatgttgagattgaagtttcatgtttgtaagcagaatgaatgttcttgtcgaagaatctcttttaagaatatctttcccacttcttcagtaaattttcctactttcttatgtcgataactgccactccttttctgagtttgcctgcactctcttggtagataggacactccctttcttagctcaggatttactttgcttgtgtaaatgagtgcttaatgtttttatttgattcattttacacgttgagatcctatagcatttaattagactagattttatcttccaatatcactaaacctagatgtcaaatatagtaaaaagttatgttgctcgaactcttcaaagatatcgacatgtgtgtgtcagatcctccaaaagtagtgcatttttggaggatccgacgggggtggggcaacaattttggagagttcgagcaacttaggtaaaaagtgtattcatgctctaaatgttgttaggttttctatctttcttatcttgtcttgtgaattttgctttcttggcaactgtttcattgtcctatatttttctcatgcaaatacctttaaaattatatactttcgtgccaaccatttaaatgaaacttaaggaagctaatcacacaggatttcaccatccaaatgtcgaaaatgaatcgcaatttgtgatagttttagacagagagcttatttttatagatgcaattagaataatagaaatgtaaaataactatcagtaaccagtaagatgccggtctttatggtctattttcagagggggagatctattgtagcttgctttcagcatcagttagtatagcataggactgttgctaacagggttcaattttaaactgtatttggtgaacataatgacatgagagctcaacttacataactacagattagagagagacattcattttatggaatggatttaagaagttcatgtttctacatttagttgttataagaacagggtggtaactggtaaccgacgcaaacatgatggctaattatcccaagtttatacaatttaatctatgtggttgctcatacatctttggtcaatgttcactttacagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaattgctcctttgaaggctattgactttggattatcagtgttctttaaacccggtaatgcaataagtaatgatagtgtgttgtaactttttttcatgctaaaaaagattaatgttggaatcatcttttaatatgtttaacaggggaacggtttaatgagattgtgggaagtccttattacatggctccagaggtcctaaaacgcaattatggcccggaggttgatgtctggagtgctggagttatcctttatattcttctttgtggtgttccacctttctgggcaggtccgtttcttttatttgccttctttaggtaacaagtaatacctcggttcaccgacaaataacaaattaaatcaaccttggtgcatactattcatttcttagaacttggtaatttattaatgagtgtcaaaatgaaatggaatttgaaacatattgatgcgttgttaacatcgtcgatgactaggtgagggtggctgcaagagtgaatcccacctcttcctggaccaattcaaaatcaaagttcctttttttccttttggcctgtgatagtcagtactttgtgcttccattaatttactcacagctttctcagtaattgcctaaaattctactgaatatgtgcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttggtaatgtttgttaatccttgagatgctatactttcatttgcttgctgtttgttctctcttagtttcataagttcaattttgtgacttttttggggctttcgtgcatatacatgaatgcagagcacacctggttacaaaatataaagaaagcaccaaatgtctcattgggtgagactgtgaaagcaaggcttaaacaattttcagtaatgaacaagctcaagaaaagagctctaacggtaaaatattcccttttcatttgcagttacatatcattagcgttctgcaccttctcagaaggatatcttcatatgcctaattcttaaagcccaaaataaggacaaaagattttctgccttctcacaaaagtttcgggtgttatattctttcttcattgcttgcacagattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgtaagcattctcatgctcttcatttcacttgattggattttctttcaactttacgaagactaaatatatatatttataagtcaagcgtctaatattattataattgttccctttttgatcttctgcttcacttttataggcggatgttgatggagatggaagcttaaattatggggagtttgttgctgtatctgttcatctcagaaaaatggcaaatgatgagcacctgcacaaagcattttcagttttcgacagagatcagagtggttacatagaaatcgaggagctgcgcagtgccttgagtgatgaggatggtggcaacagtgaggaagtcatcaatgccattatgcatgatgttgacactgacaaggttagaattttttttgtgtccattcatgtatatctactggatttcatactagtgcatatacttctcttataaatgtctctttaattgcttgtggaaccataaataccccccttatcttgttccgcagttccaactacacacgaaactatgttgaagacgtattacccccttgtacttgtaaaatgtgtatcttccgtcatcctgagaggcatatatgatgtaaccaaatgtgaacatttaagtatatgcatgtgctgcctcatgtcatttacccctttttcccttttcacttctcttccatctccgcaacatgttaatgtcctctagcaaatctgaaaaaagatggagtgagcttctcaacggaaaaaatcggattcaccctagttttagttttctggcaacataaacagctcaagtcgatacaaaaaaacaaatgttcctatcaaaaaactcaaattagtagaaaggaaaagaagggtcataggtaagggaagaacaaaaaggaatagaaagacccttcggggtggcccagttatttgggcttggggcttccatgttggaggtctcaagttcaaaaccccttgccaatgaaagcaacgggttttccttctacgttgactgtcgagctcgtcgcaccgggcttacctagtgcggtttacctctcctttgcgtgctataggaataggggttttaccctgtgtgcactcaaagggtagcgactgcggatttcccaaaagaaaaaggaagagaaaggtaaagaaaacattttaaaaaagagtaggatggttataatagaagaaatgctataatattttttcacctcaggcgctttaagagagtaaatactctacgttacatggcgtcccaatgttgtcgtagttacattttggacaagaacagggagtaataggcctcccgcaaagttaaaatgtagtagggaatgggggaataattgaggaaggtatttatatattttctcctttctaaatcatattttcttttccattcactgctcttctgttcaaccttatcattcatttggactaaatgttgaattgattttgagcttttagtttgttaccacagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 31 - DNA, 4914 bp, Solanum lycopersicum; identical to SEQ ID NO: 3
  source 1..4914: mol_type = genomic DNA

SEQ ID NO: 32 - DNA, 4914 bp, Solanum lycopersicum
  source 1..4914: mol_type = genomic DNA
atggggaattgctgtgggacacctggtaattcttctgagaataagaagaagaagaacaaaccaaacccttttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcaaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcaggtacagttttactaattaatgcttgtctttgcttttcatctataattgttacttgtttgtttatttcatgtaatttaggggttgctaatttaggattatgttaatgaagtttgaattgacaatgccaactgaaagtgtcaatttagactagaaaggaatgttgagattgaagtttcatgtttgtaagcagaatgaatgttcttgtcgaagaatctcttttaagaatatctttcccacttcttcagtaaattttcctactttcttatgtcgataactgccactccttttctgagtttgcctgcactctcttggtagataggacactccctttcttagctcaggatctactttgcttgtgtaaatgagtgcttaatgtttttatttgattcattttacacgttgagatcctatagcatttaattagactagattttatcttccaatatcactaaacctagatgtcaaatatagtaaaaagttatgttgctcgaactcttcaaagatatcgacatgtgtgtgtcagatcctccaaaagtagtgcatttttggaggatccgacgggggtggggcaacaattttggagagttcgagcaacttaggtaaaaagtgtattcatgctctaaatgttgttaggttttctatctttcttatcttgtcttgtgaattttgctttcttggcaactgtttcattgtcctatatttttctcatgcaaatacctttaaaattatatactttcgtgccaaccatttaaatgaaacttaaggaagctaatcacacaggatttcaccatccaaatgtcgaaaatgaatcgcaatttgtgatagttttagacagagagcttatttttatagatgcaattagaataatagaaatgtaaaataactatcagtaaccagtaagatgccggtctttatggtctattttcagagggggagatctattgtagcttgctttcagcatcagttagtatagcataggactgttgctaacagggttcaattttaaactgtatttggtgaacataatgacatgagagctcaacttacataactacagattagagagagacattcattttatggaatggatttaagaagttcatgtttctacatttagttgttataagaacagggtggtaactggtaaccgacgcaaacatgatggctaattatcccaagtttatacaatttaatctatgtggttgctcatacatctttggtcaatgttcactttacagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaactgctcctttgaaggctattgactttggattatcagtgttctttaaacccggtaatgcaataagtaatgatagtgtgttgtaactttttttcatgctaaaaaagattaatgttggaatcatcttttaatatgtttaacaggggaacgctttaatgagattgtgggaagtccttattacatggctccagaggtcctaaaacgcaattatggcccggaggttgatgtctggagtgctggagttatcctttatattcttctttgtggtgttccacctttctgggcaggtccgtttcttttatttgccttctttaggtaacaagtaatacctcggttcaccgacaaataacaaagtaaatcaaccttggtgcatactattcatttcttagaacttggtaatttattaatgagtgtcaaaatgaaatggaatttgaaacatattgatgcgttgttaacatcgtcgatgactaggtgagggtggctgcaagagtgaatcccacctcttcctggaccaattcaaaatcaaagttcctttttttccttttggcctgtgatagtcagtactttgtgcttccattaatttactcacagctttctcagtaattgcctaaagttctactgaatatgtgcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttggtaatgtttgttaatccttgagatgctatactttcatttgcttgctgtttgttctctcttagtttcataagttcaattttgtgacttttttggggctttcgtgcatatacatgaatgcagagcacacctggttacaaaatataaagaaagcaccaaatgtctcattgggtgagactgtgaaagcaaggcttaaacaattttcagtaatgaacaagctcaagaaaagagctctaacggtaaaatattcccttttcatttgcagttacatatcattagcgttctgcaccttctcagaaggatatcttcatatgcctaattcttaaagcccaaaataaggacaaaagattttctgccttctcacaaaagtttcgggtgttatattctttcttcattgcttgcacagattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgtaagcattctcatgctcttcatttcacttgattggattttctttcaactttacgaagactaaatatatatatttataagtcaagcgtctaatattattataattgttcccttttttatcttctgcttcacttttataggcggatgttgatggagatggaagcttaaattatggggagtttgttgctgtatctgttcatctcagaaaaatggcaaatgatgagcacctgcacaaagcattttcagtttttgacagagatcagagtggttacatagaaatcgaggagctgcgcagtgccttgagtgatgaggatggtggcaacagtgcggaagtcatcaatgccattatgcatgatgttgacactgacaaggttagaattttttttgtgtccattcatgtatatctactggatttcatactagtgcatatacttctcttataaatgtctctttaattgcttgtggaaccataaataccccccttatcttgttccgcagctccaactacacacgaaactatgttgaagacgtattacccccttgtacttgtaaaatgtgtatcttccgtcatcctgagaggcatatatgatgtaaccaaatgtgaacatttaagtatatgcatgtgctgcctcatgtcatttacccctttttcccttttcacttctcttccatctccgcaacatgttaatgtcctctagcaaatctgaaaaaagatggagtgagcttctcaacggaaaaaatcggattcaccctagttttagttttctggcaacataaacagctcaagtcgatacaaaaaaacaaatgttcctatcaaaaaactcaaattagtagaaaggaaaagaagggtcataagtaagggaagaacaaaaaggaatagaaagacccttcggggtggcccagttatttgggcttggggcttggggcttgggacttccatgttggaggtctcaagttcaaaaccccttgccaatgaaagcaacgggttttccttctacgttgactgtcgagctcgtcgcaccgggcttacctagtgcggtttacctctcctttgcgtgctataggaataggggttttaccctgtgtgcactcaaagggtagcgactgcggatttcccaaaagaaaaaggaagagaaaggtaaagaaaacattttaaaaaagagtaggatggttataatagaagaaatgctacaatattttttcacctcaggcgctttaagagagtaaatactctacgttacatggcgtcccaatgttgtcgtagttacattttggacaagaacagggagtaataggcctcccgcaaagttaaaatgtagtggggaatgggggaataattgaggaaggtatttatatattttctcctttctaaatcatattttcttttccattcactgctcttctgttcaaccttatcattcatttggactaaatgttgaattgattttgagcttttagtttgttaccacagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 33 - DNA, 4914 bp, Solanum lycopersicum; identical to SEQ ID NO: 3
  source 1..4914: mol_type = genomic DNA

SEQ ID NO: 34 - DNA, 4897 bp, Solanum lycopersicum
  source 1..4897: mol_type = genomic DNA
atggggaattgctgtgggacacctggtaattcttctgagaataagaagaagaacaaaccaaacccgtttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcgaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcaggtacagttttactaattgatgcttgtctttgcttttcatctataattgttacttgtttgtttatttcatgtaatttaggggttgctaatttaggattatgttaatgaagtttgaattgacaatgccaactgaaagtatcaatttagactagaaaggaatgttgagattgaagtttcatgtttgtaagcagaatgaatgttcttgtcgaagaatctcttttaagaatatctttcccacttcttcagtaaattttcctactttcttatgtcgataactgccactccttttctgagtttgcctgcactctcttggtagataggacactccctttcttagctcaggatttactttgcttgtgtaaatgagtgcttaatgtttttatttgattcattttacacgttgagatcctatagcatttaattagactagattttatcttccaatatcactaaacctagatgtcaaatatagtaaaaagttatgttgctcgaactcttcaaagatatcgacatgtgtgtgtcagatcctccaaaagtagtgcatttttggaggatccgacgggggtggggcaacaattttggagagttcgagcaacttaggtaaaaagtgtattcatgctctaaatgttgttaggttttctatctttcttatcttgtcttgtgaattttgctttcttggcaactgtttcattgtcctatatttttctcatgcaaatacctttaaaattatattctttcgtgccaaccatttaaatgaaacttaaggaagctaatcacacaggatttcaccatccaaatgtcgaaaatgaatcgcaatttgtgatagttttagacagagagcttatttttatagatgcaattagaataatagaaatgtaaaataactatcagtaaccagtaagatgccggtctttatggtctattttcagagggggagatctattgtagcttgctttcagcatcagttagtatagcataggactgttgctaacagggttcaattttaaactgtatttggtgaacataatgacatgagagctcaacttacataactacagattagagagagacattcattttatggaatggatttaagaagttcatgtttctacatttagttgttataagaacagggtggtaactggtaaccgacgcaaacatgatggctaattatcccaagtttatacaatttaatctatgtggttgctcatacatctttggtcaatgttcactttacagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaattgctcctttgaaggctattgactttggattatcagtgttctttaaacccggtaatgcaataagtaatgatagtgtgttgtaactttttttcatgctaaaaaagattaatgttggaatcatcttttaatatgtttaacaggggaacggtttaatgagattgtgggaagtccttattacatggctccagaggtcctaaaacgcaattatggcccggaggttgatgtctggagtgctggagttatcctttatattcttctttgtggtgttccacctttctgggcaggtccgtttcttttatttgccttctttaggtaacaagtaatacctcggttcaccgacaaataacaaattaaatcaaccttggtgcatactattcatttcttagaacttggtaatttattaatgagtgtcaaaatgaaatggaatttgaaacatattgatgcgttgttaacatcgtcgatgactaggtgagggtggctgcaagagtgaatcccacctcttcctggaccaattcaaaatcaaagttcctttttttccttttggcctgtgatagtcagtactttgtgcttccattaatttactcacagctttctcagtaattgcctaaaattctactgaatatgtgcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttggtaatgtttgttaatccttgagatgctatactttcatttgcttgctgtttgttctctcttagtttcataagttcaattttgtgacttttttggggctttcgtgcatatacatgaatgcagagcacacctggttacaaaatataaagaaagcaccaaatgtctcattgggtgagactgtgaaagcaaggcttaaacaattttcagtaatgaacaagctcaagaaaagagctctaacggtaaaatattcccttttcatttgcagttacatatcattagcgttctgcaccttctcagaaggatatcttcatatgcctaattcttaaagcccaaaataaggacaaaagattttctgccttctcacaaaagtttcgggtgttatattctttcttcattgcttgcacagattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgtaagcattctcatgctcttcatttcacttgattggattttctttcaactttacgaagactaaatatatatatttataagtcaagcgtctaatattattataattgttccctttttgatcttctgcttcacttttataggcggatgttgatggagatggaagcttaaattatggggagtttgttgctgtatctgttcatctcagaaaaatggcaaatgatgagcacctgcacaaagcattttcagttttcgacagagatcagagtggttacatagaaatcgaggagctgcgcagtgccttgagtgatgaggatggtggcaacagtgaggaagtcatcaatgccattatgcatgatgttgacactgacaaggttagaattttttttgtgtccattcatgtatatctactggatttcatactagtgcatatacttctcttataaatgtctctttaattgcttgtggaaccataaataccccccttatcttgttccgcagttccaactacacacgaaactatgttgaagacgtattacccccttgtacttgtaaaatgtgtatcttccgtcatcctgagaggcatatatgatgtaaccaaatgtgaacatttaagtatatgcatgtgctgcctcatgtcatttacccctttttcccttttcacttctcttccatctccgcaacatgttaatgtcctctagcaaatctgaaaaaagatggagtgagcttctcaacggaaaaaatcggattcaccctagttttagttttctggcaacataaacagctcaagtcgatacaaaaaaacaaatgttcctatcaaaaaactcaaattagtagaaaggaaaagaagggtcataggtaagggaagaacaaaaaggaatagaaagacccttcggggtggcccagttatttgggcttggggcttccatgttggaggtctcaagttcaaaaccccttgccaatgaaagcaacgggttttccttctacgttgactgtcgagctcgtcgcaccgggcttacctagtgcggtttacctctcctttgcgtgctataggaataggggttttaccctgtgtgcactcaaagggtagcgactgcggatttcccaaaagaaaaaggaagagaaaggtaaagaaaacattttaaaaaagagtaggatggttataatagaagaaatgctataatattttttcacctcaggcgctttaagagagtaaatactctacgttacatggcgtcccaatgttgtcgtagttacattttggacaagaacagggagtaataggcctcccgcaaagttaaaatgtagtagggaatgggggaataattgaggaaggtatttatatattttctcctttctaaatcatattttcttttccattcactgctcttctgttcaaccttatcattcatttggactaaatgttgaattgattttgagcttttagtttgttaccacagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 35 - DNA, 4897 bp, Solanum lycopersicum; identical to SEQ ID NO: 34
  source 1..4897: mol_type = genomic DNA

SEQ ID NO: 36 - DNA, 4914 bp, Solanum lycopersicum; identical to SEQ ID NO: 3
  source 1..4914: mol_type = genomic DNA

SEQ ID NO: 37 - DNA, 1602 bp, Solanum lycopersicum; identical to SEQ ID NO: 1
  source 1..1602: mol_type = other DNA

SEQ ID NO: 38 - DNA, 1638 bp, Solanum lycopersicum
  source 1..1638: mol_type = other DNA
atggggaattgctgtgggacacctggtaattcttctgagaataagaagaagaacaaaccaaacccgtttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcgaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttgttatgaagactatagtagaagttgttcagatgtgtcatatgcatggagtaatgcatcgtgatctcaaacctgagaactttctgtttggtaacaagaaagaaattgctcctttgaaggctattgactttggattatcagtgttctttaaacccggggaacggtttaatgagattgtgggaagtccttattacatggctccagaggtcctaaaacgcaattatggcccggaggttgatgtctggagtgctggagttatcctttatattcttctttgtggtgttccacctttctgggcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttgagcacacctggttacaaaatataaagaaagcaccaaatgtctcattgggtgagactgtgaaagcaaggcttaaacaattttcagtaatgaacaagctcaagaaaagagctctaacgattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgcggatgttgatggagatggaagcttaaattatggggagtttgttgctgtatctgttcatctcagaaaaatggcaaatgatgagcacctgcacaaagcattttcagttttcgacagagatcagagtggttacatagaaatcgaggagctgcgcagtgccttgagtgatgaggatggtggcaacagtgaggaagtcatcaatgccattatgcatgatgttgacactgacaagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 39 - DNA, 1641 bp, Solanum lycopersicum
  source 1..1641: mol_type = other DNA
atggggaattgctgtgggacacctggtaattcttctgagaataagaagaagaagaacaaaccaaacccttttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcaaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcagatgtgtcatatgcatggagtaatgcatcgtgatctcaaacctgagaactttctgtttggtaacaagaaagaaactgctcctttgaaggctattgactttggattatcagtgttctttaaacccggggaacgctttaatgagattgtgggaagtccttattacatggctccagaggtcctaaaacgcaattatggcccggaggttgatgtctggagtgctggagttatcctttatattcttctttgtggtgttccacctttctgggcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttgagcacacctggttacaaaatataaagaaagcaccaaatgtctcattgggtgagactgtgaaagcaaggcttaaacaattttcagtaatgaacaagctcaagaaaagagctctaacgattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgcggatgttgatggagatggaagcttaaattatggggagtttgttgctgtatctgttcatctcagaaaaatggcaaatgatgagcacctgcacaaagcattttcagtttttgacagagatcagagtggttacatagaaatcgaggagctgcgcagtgccttgagtgatgaggatggtggcaacagtgaggaagtcatcaatgccattatgcatgatgttgacactgacaagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 40 - DNA, 1641 bp, Solanum lycopersicum; identical to SEQ ID NO: 39
  source 1..1641: mol_type = other DNA

SEQ ID NO: 41 - DNA, 1602 bp, Solanum lycopersicum; identical to SEQ ID NO: 1
  source 1..1602: mol_type = other DNA

SEQ ID NO: 42 - DNA, 1713 bp, Solanum lycopersicum
  source 1..1713: mol_type = other DNA
atggggaattgctgtgggacacctggtaattcttctgagaataagaagaagaacaaaccaaacccgtttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcgaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaattgctcctttgaaggctattgactttggattatcagtgttctttaaacccggggaacggtttaatgagattgtgggaagtccttattacatggctccagaggtcctaaaacgcaattatggcccggaggttgatgtctggagtgctggagttatcctttatattcttctttgtggtgttccacctttctgggcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttgagcacacctggttacaaaatataaagaaagcaccaaatgtctcattgggtgagactgtgaaagcaaggcttaaacaattttcagtaatgaacaagctcaagaaaagagctctaacgattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgcggatgttgatggagatggaagcttaaattatggggagtttgttgctgtatctgttcatctcagaaaaatggcaaatgatgagcacctgcacaaagcattttcagttttcgacagagatcagagtggttacatagaaatcgaggagctgcgcagtgccttgagtgatgaggatggtggcaacagtgaggaagtcatcaatgccattatgcatgatgttgacactgacaagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 43 - DNA, 1641 bp, Solanum lycopersicum
  source 1..1641: mol_type = other DNA
atggggaattgctgtgggacacctggtaattcttctgagaataagaagaagaagaacaaaccaaacccttttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcaaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcagatgtgtcatatgcatggagtaatgcatcgtgatctcaaacctgagaactttctgtttggtaacaagaaagaaactgctcctttgaaggctattgactttggattatcagtgttctttaaacccggggaacgctttaatgagattgtgggaagtccttattacatggctccagaggtcctaaaacgcaattatggcccggaggttgatgtctggagtgctggagttatcctttatattcttctttgtggtgttccacctttctgggcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttgagcacacctggttacaaaatataaagaaagcaccaaatgtctcattgggtgagactgtgaaagcaaggcttaaacaattttcagtaatgaacaagctcaagaaaagagctctaacgattatggccgagtttttatcagcggaagaagtggctggaatgaaggacgcatttgatatgatggatacaggaaagaaaggcaagattaaccttggagaacttaaaaatggtctgcaaaagcttggccatcagatacctgatgttgatcttcagattcttatggaagctgcggatgttgatggagatggaagcttaaattatggggagtttgttgctgtatctgttcatctcagaaaaatggcaaatgatgagcacctgcacaaagcattttcagtttttgacagagatcagagtggttacatagaaatcgaggagctgcgcagtgccttgagtgatgaggatggtggcaacagtgaggaagtcatcaatgccattatgcatgatgttgacactgacaagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 44 - DNA, 1602 bp, Solanum lycopersicum; identical to SEQ ID NO: 1
  source 1..1602: mol_type = other DNA

SEQ ID NO: 45 - DNA, 1602 bp, Solanum lycopersicum; identical to SEQ ID NO: 1
  source 1..1602: mol_type = other DNA

SEQ ID NO: 46 - DNA, 1599 bp, Solanum lycopersicum
  source 1..1599: mol_type = other DNA
atggggaattgctgtgggacacctggtaattcttctgagaataagaagaagaacaaaccaaacccgtttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcgaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcagatgtgtcatatgcatggagtaatgcatcgtgatctcaaacctgagaactttctgtttggtaacaagaaagaaattgctcctttgaaggctattgactttggattatcagtgttctttaaacccggggaacggtttaatgagattgtgggaagtccttattacatggctccagaggtcctaaaacgcaattatggcccggaggttgatgtctggagtgctggagttatcctttatattcttctttgtggtgttccacctttctgggcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttgagcacacctggttacaaaatataaagaaagcaccaaatgtctcattgggtgagactgtgaaagcaaggcttaaacaattttcagtaatgaacaagctcaagaaaagagctctaacgattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgcggatgttgatggagatggaagcttaaattatggggagtttgttgctgtatctgttcatctcagaaaaatggcaaatgatgagcacctgcacaaagcattttcagttttcgacagagatcagagtggttacatagaaatcgaggagctgcgcagtgccttgagtgatgaggatggtggcaacagtgaggaagtcatcaatgccattatgcatgatgttgacactgacaaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 47 - DNA, 1599 bp, Solanum lycopersicum
  source 1..1599: mol_type = other DNA
atggggaattgctgtgggacacctggtaattcttctgagaataagaagaagaacaaaccaaacccgtttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcgaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcagatgtgtcatatgcatggagtaatgcatcgtgatctcaaacctgagaactttctgtttggtaacaagaaagaaattgctcctttgaaggctattgactttggattatcagtgttctttaaacccggggaacggtttaatgagattgtgggaagtccttattacatggctccagaggtcctaaaacgcaattatggcccggaggttgatgtctggagtgctggagttatcctttatattcttctttgtggtgttccacctttctgggcagagactgaacaaggagtagcccaagcaattattcggtctgtggttgatttcaagagagatccatggcctaaggtttctgataatgcaaaggatcttgtaaagaagatgcttgacccagatccaactcgacgactcacagctcagcaggttcttgagcacacctggttacaaaatataaagaaagcaccaaatgtctcattgggtgagactgtgaaagcaaggcttaaacaattttcagtaatgaacaagctcaagaaaagagctctaacgattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgcggatgttgatggagatggaagcttaaattatggggagtttgttgctgtatctgttcatctcagaaaaatggcaaatgatgagcacctgcacaaagcattttcagttttcgacagagatcagagtggttacatagaaatcgaggagctgcgcagtgccttgagtgatgaggatggtggcaacagtgaggaagtcatcaatgccattatgcatgatgttgacactgacaaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 48 - DNA, 1713 bp, Solanum lycopersicum; identical to SEQ ID NO: 42
  source 1..1713: mol_type = other DNA

SEQ ID NO: 49 - DNA, 1677 bp, Solanum lycopersicum
  source 1..1677: mol_type = other DNA
atggggaattgctgtgggacacctggtaattcttctgagaataagaagaagaagaacaaaccaaacccttttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcaaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaactgctcctttgaaggctattgactttggattatcagtgttctttaaacccggggaacgctttaatgagattgtgggaagtccttattacatggctccagaggtcctaaaacgcaattatggcccggaggttgatgtctggagtgctggagttatcctttatattcttctttgtggtgttccacctttctgggcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttgagcacacctggttacaaaatataaagaaagcaccaaatgtctcattgggtgagactgtgaaagcaaggcttaaacaattttcagtaatgaacaagctcaagaaaagagctctaacgattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgcggatgttgatggagatggaagcttaaattatggggagtttgttgctgtatctgttcatctcagaaaaatggcaaatgatgagcacctgcacaaagcattttcagtttttgacagagatcagagtggttacatagaaatcgaggagctgcgcagtgccttgagtgatgaggatggtggcaacagtgcggaagtcatcaatgccattatgcatgatgttgacactgacaaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 50 - DNA, 1716 bp, Solanum lycopersicum
  source 1..1716: mol_type = other DNA
atggggaattgctgtgggacacctggtaattcttctgagaataagaagaagaagaacaaaccaaacccttttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcaaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaactgctcctttgaaggctattgactttggattatcagtgttctttaaacccggggaacgctttaatgagattgtgggaagtccttattacatggctccagaggtcctaaaacgcaattatggcccggaggttgatgtctggagtgctggagttatcctttatattcttctttgtggtgttccacctttctgggcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttgagcacacctggttacaaaatataaagaaagcaccaaatgtctcattgggtgagactgtgaaagcaaggcttaaacaattttcagtaatgaacaagctcaagaaaagagctctaacgattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgcggatgttgatggagatggaagcttaaattatggggagtttgttgctgtatctgttcatctcagaaaaatggcaaatgatgagcacctgcacaaagcattttcagtttttgacagagatcagagtggttacatagaaatcgaggagctgcgcagtgccttgagtgatgaggatggtggcaacagtgaggaagtcatcaatgccattatgcatgatgttgacactgacaagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 51 - DNA, 1641 bp, Solanum lycopersicum; identical to SEQ ID NO: 39
  source 1..1641: mol_type = other DNA

SEQ ID NO: 52 - DNA, 1677 bp, Solanum lycopersicum
  source 1..1677: mol_type = other DNA
atggggaattgctgtgggacacctggtaattcttctgagaataagaagaagaagaacaaaccaaacccttttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcaaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaactgctcctttgaaggctattgactttggattatcagtgttctttaaacccggggaacgctttaatgagattgtgggaagtccttattacatggctccagaggtcctaaaacgcaattatggcccggaggttgatgtctggagtgctggagttatcctttatattcttctttgtggtgttccacctttctgggcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttgagcacacctggttacaaaatataaagaaagcaccaaatgtctcattgggtgagactgtgaaagcaaggcttaaacaattttcagtaatgaacaagctcaagaaaagagctctaacgattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgcggatgttgatggagatggaagcttaaattatggggagtttgttgctgtatctgttcatctcagaaaaatggcaaatgatgagcacctgcacaaagcattttcagtttttgacagagatcagagtggttacatagaaatcgaggagctgcgcagtgccttgagtgatgaggatggtggcaacagtgaggaagtcatcaatgccattatgcatgatgttgacactgacaaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 53 - DNA, 1677 bp, Solanum lycopersicum; identical to SEQ ID NO: 52
  source 1..1677: mol_type = other DNA

SEQ ID NO: 54 - DNA, 1674 bp, Solanum lycopersicum
  source 1..1674: mol_type = other DNA
atggggaattgctgtgggacacctggtaattcttctgagaataagaagaagaacaaaccaaacccgtttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcgaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcagttgcattatgtttcctcaagaaatggctgttcttctttcaattttcaacttacggagtttcaattgacattgcagatgtgtcatatgcatggagtaatgcatcgtgatctcaaacctgagaactttctgtttggtaacaagaaagaaattgctcctttgaaggctattgactttggattatcagtgttctttaaacccggggaacggtttaatgagattgtgggaagtccttattacatggctccagaggtcctaaaacgcaattatggcccggaggttgatgtctggagtgctggagttatcctttatattcttctttgtggtgttccacctttctgggcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttgagcacacctggttacaaaatataaagaaagcaccaaatgtctcattgggtgagactgtgaaagcaaggcttaaacaattttcagtaatgaacaagctcaagaaaagagctctaacgattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgcggatgttgatggagatggaagcttaaattatggggagtttgttgctgtatctgttcatctcagaaaaatggcaaatgatgagcacctgcacaaagcattttcagttttcgacagagatcagagtggttacatagaaatcgaggagctgcgcagtgccttgagtgatgaggatggtggcaacagtgaggaagtcatcaatgccattatgcatgatgttgacactgacaaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 55 - DNA, 1677 bp, Solanum lycopersicum; identical to SEQ ID NO: 52
  source 1..1677: mol_type = other DNA

SEQ ID NO: 56 - DNA, 1713 bp, Solanum lycopersicum; identical to SEQ ID NO: 42
  source 1..1713: mol_type = other DNA

SEQ ID NO: 57 - DNA, 1641 bp, Solanum lycopersicum
  source 1..1641: mol_type = other DNA
atggggaattgctgtgggacacctggtaattcttctgagaataagaagaagaagaacaaaccaaacccttttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcaaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcagatgtgtcatatgcatggagtaatgcatcgtgatctcaaacctgagaactttctgtttggtaacaagaaagaaactgctcctttgaaggctattgactttggattatcagtgttctttaaacccggggaacgctttaatgagattgtgggaagtccttattacatggctccagaggtcctaaaacgcaattatggcccggaggttgatgtctggagtgctggagttatcctttatattcttctttgtggtgttccacctttctgggcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttgagcacacctggttacaaaatataaagaaagcaccaaatgtctcattgggtgagactgtgaaagcaaggcttaaacaattttcagtaatgaacaagctcaagaaaagagctctaacgattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgcggatgttgatggagatggaagcttaaattatggggagtttgttgctgtatctgttcatctcagaaaaatggcaaatgatgagcacctgcacaaagcattttcagtttttgacagagatcagagtggttacatagaaatcgaggagctgcgcagtgccttgagtgatgaggatggtggcaacagtgcggaagtcatcaatgccattatgcatgatgttgacactgacaagcttcaaagttcatcagttacattgtctataaccatgcaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 58 - DNA, 1677 bp, Solanum lycopersicum; identical to SEQ ID NO: 52
  source 1..1677: mol_type = other DNA

SEQ ID NO: 59 - DNA, 1602 bp, Solanum lycopersicum
  source 1..1602: mol_type = other DNA
atggggaattgctgtgggacacctggtaattcttctgagaataagaagaagaagaacaaaccaaacccttttgctcttgattatggtgcaactcaagcatctggaggtgatggaaacaagcttgttgtgttgaaagatccaacaggacacaatattcaagaaaaatatgatcttggttgtgagctaggaagaggagaatttggggttacatatttatgtactgatgttgatacaggggacaaatatgcttgcaaatcgatatcaaagaagaaacttaggactgctgtagatatagatgatgttaggagagaagttgagatcatgaagcatttgcctaaacatcctaatattgtgaccttgaaggacacttatgaggatgataatgcagtgcatattgtgatggaactttgtgaggggggtgagttgtttgataggattgttgccaggggacattatacagagagagcagctgcggttattatgaagactatagtagaagttgttcagatgtgtcatatgcatggagtaatgcatcgtgatctcaaacctgagaactttctgtttggtaacaagaaagaaactgctcctttgaaggctattgactttggattatcagtgttctttaaacccggggaacgctttaatgagattgtgggaagtccttattacatggctccagaggtcctaaaacgcaattatggcccggaggttgatgtctggagtgctggagttatcctttatattcttctttgtggtgttccacctttctgggcagagactgaacaaggagtagcccaagcaattattcggtctgtggttgatttcaagagagatccatggcctaaggtttctgataatgcaaaggatcttgtaaagaagatgcttgacccggatccaactcgacgactcacagctcagcaggttcttgagcacacctggttacaaaatataaagaaagcaccaaatgtctcattgggtgagactgtgaaagcaaggcttaaacaattttcagtaatgaacaagctcaagaaaagagctctaacgattatggccgagtttttatcagcggaagaagtggctggaatgaaggacgcatttgatatgatggatacaggaaagaaaggcaagattaaccttggagaacttaaaaatggtctgcaaaagcttggccatcagatccctgatgttgatcttcagattcttatggaagctgcggatgttgatggagatggaagcttaaattatggggagtttgttgctgtatctgttcatctcagaaaaatggcaaatgatgagcacctgcacaaagcattttcagtttttgacagagatcagagtggttacatagaaatcgaggagctgcgcagtgccttgagtgatgaggatggtggcaacagtgcggaagtcatcaatgccattatgcatgatgttgacactgacaaggatggccgcattagttacgaggaatttgctgcaatgatgaaggctggcacagattggagaaaagcatcgagacagtattctcgtgaaagattcaacagtctcagcttaaaattgatgagagatggctcgatacaagtcggaaaggaagaaggtagatga

SEQ ID NO: 60 - DNA, 1713 bp, Solanum lycopersicum; identical to SEQ ID NO: 42
  source 1..1713: mol_type = other DNA

SEQ ID NO: 61 - DNA, 1641 bp, Solanum lycopersicum; identical to SEQ ID NO: 39
  source 1..1641: mol_type = other DNA

SEQ ID NO: 62 - DNA, 1713 bp, Solanum lycopersicum; identical to SEQ ID NO: 42
  source 1..1713: mol_type = other DNA

SEQ ID NO: 63 - DNA, 1677 bp, Solanum lycopersicum; identical to SEQ ID NO: 52
  source 1..1677: mol_type = other DNA

SEQ ID NO: 64 - DNA, 1713 bp, Solanum lycopersicum; identical to SEQ ID NO: 42
  source 1..1713: mol_type = other DNA

SEQ ID NO: 65 - DNA, 1677 bp, Solanum lycopersicum; identical to SEQ ID NO: 52
  source 1..1677: mol_type = other DNA

SEQ ID NO: 66 - protein, 545 aa, Solanum lycopersicum
MGNCCGTPGNSSENKKKNKPNPFALDYGATQASGGDGNKLVVLKDPTGHNIQEKYDLGCELGRGEFGVTYLCTDVDTGDKYACKSISKKKLRTAVDIDDVRREVEIMKHLPKHPNIVTLKDTYEDDNAVHIVMELCEGGELFDRIVARGHYTERAAAVVMKTIVEVVQMCHMHGVMHRDLKPENFLFGNKKEIAPLKAIDFGLSVFFKPGERFNEIVGSPYYMAPEVLKRNYGPEVDVWSAGVILYILLCGVPPFWAETEQGVAQAIIRSVVDFKRDPWPKVSDNAKDLVKKMLDPDPTRRLTAQQVLEHTWLQNIKKAPNVSLGETVKARLKQFSVMNKLKKRALTIMAEFLSAEEVAGMKDAFDMMDTGKKGKINLGELKNGLQKLGHQIPDVDLQILMEAADVDGDGSLNYGEFVAVSVHLRKMANDEHLHKAFSVFDRDQSGYIEIEELRSALSDEDGGNSEEVINAIMHDVDTDKLQSSSVTLSITMQDGRISYEEFAAMMKAGTDWRKASRQYSRERFNSLSLKLMRDGSIQVGKEEGR

SEQ ID NO: 67 - protein, 571 aa, Solanum lycopersicum
MGNCCGTPGNSSENKKKKNKPNPFALDYGATQASGGDGNKLVVLKDPTGHNIQEKYDLGCELGRGEFGVTYLCTDVDTGDKYACKSISKKKLRTAVDIDDVRREVEIMKHLPKHPNIVTLKDTYEDDNAVHIVMELCEGGELFDRIVARGHYTERAAAVIMKTIVEVVQLHYVSSRNGCSSFNFQLTEFQLTLQMCHMHGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVVDFKRDPWPKVSDNAKDLVKKMLDPDPTRRLTAQQVLEHTWLQNIKKAPNVSLGETVKARLKQFSVMNKLKKRALTIMAEFLSAEEVAGMKDAFDMMDTGKKGKINLGELKNGLQKLGHQIPDVDLQILMEAADVDGDGSLNYGEFVAVSVHLRKMANDEHLHKAFSVFDRDQSGYIEIEELRSALSDEDGGNSEEVINAIMHDVDTDKLQSSSVTLSITMQDGRISYEEFAAMMKAGTDWRKASRQYSRERFNSLSLKLMRDGSIQVGKEEGR

SEQ ID NO: 68 - protein, 571 aa, Solanum lycopersicum; identical to SEQ ID NO: 67

SEQ ID NO: 69 - protein, 533 aa, Solanum lycopersicum; identical to SEQ ID NO: 2

SEQ ID NO: 70 - protein, 570 aa, Solanum lycopersicum
MGNCCGTPGNSSENKKKNKPNPFALDYGATQASGGDGNKLVVLKDPTGHNIQEKYDLGCELGRGEFGVTYLCTDVDTGDKYACKSISKKKLRTAVDIDDVRREVEIMKHLPKHPNIVTLKDTYEDDNAVHIVMELCEGGELFDRIVARGHYTERAAAVIMKTIVEVVQLHYVSSRNGCSSFNFQLTEFQLTLQMCHMHGVMHRDLKPENFLFGNKKEIAPLKAIDFGLSVFFKPGERFNEIVGSPYYMAPEVLKRNYGPEVDVWSAGVILYILLCGVPPFWAETEQGVAQAIIRSVVDFKRDPWPKVSDNAKDLVKKMLDPDPTRRLTAQQVLEHTWLQNIKKAPNVSLGETVKARLKQFSVMNKLKKRALTIMAEFLSAEEVAGMKDAFDMMDTGKKGKINLGELKNGLQKLGHQIPDVDLQILMEAADVDGDGSLNYGEFVAVSVHLRKMANDEHLHKAFSVFDRDQSGYIEIEELRSALSDEDGGNSEEVINAIMHDVDTDKLQSSSVTLSITMQDGRISYEEFAAMMKAGTDWRKASRQYSRERFNSLSLKLMRDGSIQVGKEEGR

SEQ ID NO: 71 - protein, 546 aa, Solanum lycopersicum
MGNCCGTPGNSSENKKKKNKPNPFALDYGATQASGGDGNKLVVLKDPTGHNIQEKYDLGCELGRGEFGVTYLCTDVDTGDKYACKSISKKKLRTAVDIDDVRREVEIMKHLPKHPNIVTLKDTYEDDNAVHIVMELCEGGELFDRIVARGHYTERAAAVIMKTIVEVVQMCHMHGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVVDFKRDPWPKVSDNAKDLVKKMLDPDPTRRLTAQQVLEHTWLQNIKKAPNVSLGETVKARLKQFSVMNKLKKRALTIMAEFLSAEEVAGMKDAFDMMDTGKKGKINLGELKNGLQKLGHQIPDVDLQILMEAADVDGDGSLNYGEFVAVSVHLRKMANDEHLHKAFSVFDRDQSGYIEIEELRSALSDEDGGNSEEVINAIMHDVDTDKLQSSSVTLSITMQDGRISYEEFAAMMKAGTDWRKASRQYSRERFNSLSLKLMRDGSIQVGKEEGR

SEQ ID NO: 72 - protein, 571 aa, Solanum lycopersicum; identical to SEQ ID NO: 67

SEQ ID NO: 73 - protein, 533 aa, Solanum lycopersicum; identical to SEQ ID NO: 2

SEQ ID NO: 74 - protein, 532 aa, Solanum lycopersicum
MGNCCGTPGNSSENKKKNKPNPFALDYGATQASGGDGNKLVVLKDPTGHNIQEKYDLGCELGRGEFGVTYLCTDVDTGDKYACKSISKKKLRTAVDIDDVRREVEIMKHLPKHPNIVTLKDTYEDDNAVHIVMELCEGGELFDRIVARGHYTERAAAVIMKTIVEVVQMCHMHGVMHRDLKPENFLFGNKKEIAPLKAIDFGLSVFFKPGERFNEIVGSPYYMAPEVLKRNYGPEVDVWSAGVILYILLCGVPPFWAETEQGVAQAIIRSVVDFKRDPWPKVSDNAKDLVKKMLDPDPTRRLTAQQVLEHTWLQNIKKAPNVSLGETVKARLKQFSVMNKLKKRALTIMAEFLSAEEVAGMKDAFDMMDTGKKGKINLGELKNGLQKLGHQIPDVDLQILMEAADVDGDGSLNYGEFVAVSVHLRKMANDEHLHKAFSVFDRDQSGYIEIEELRSALSDEDGGNSEEVINAIMHDVDTDKDGRISYEEFAAMMKAGTDWRKASRQYSRERFNSLSLKLMRDGSIQVGKEEGR

SEQ ID NO: 75 - protein, 571 aa, Solanum lycopersicum; identical to SEQ ID NO: 67

SEQ ID NO: 76 - protein, 532 aa, Solanum lycopersicum; identical to SEQ ID NO: 74

SEQ ID NO: 77 - protein, 570 aa, Solanum lycopersicum; identical to SEQ ID NO: 70

SEQ ID NO: 78 - protein, 558 aa, Solanum lycopersicum
MGNCCGTPGNSSENKKKKNKPNPFALDYGATQASGGDGNKLVVLKDPTGHNIQEKYDLGCELGRGEFGVTYLCTDVDTGDKYACKSISKKKLRTAVDIDDVRREVEIMKHLPKHPNIVTLKDTYEDDNAVHIVMELCEGGELFDRIVARGHYTERAAAVIMKTIVEVVQLHYVSSRNGCSSFNFQLTEFQLTLQMCHMHGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVVDFKRDPWPKVSDNAKDLVKKMLDPDPTRRLTAQQVLEHTWLQNIKKAPNVSLGETVKARLKQFSVMNKLKKRALTIMAEFLSAEEVAGMKDAFDMMDTGKKGKINLGELKNGLQKLGHQIPDVDLQILMEAADVDGDGSLNYGEFVAVSVHLRKMANDEHLHKAFSVFDRDQSGYIEIEELRSALSDEDGGNSAEVINAIMHDVDTDKDGRISYEEFAAMMKAGTDWRKASRQYSRERFNSLSLKLMRDGSIQVGKEEGR

SEQ ID NO: 79 - protein, 571 aa, Solanum lycopersicum; identical to SEQ ID NO: 67

SEQ ID NO: 80 - protein, 546 aa, Solanum lycopersicum; identical to SEQ ID NO: 71

SEQ ID NO: 81 - protein, 558 aa, Solanum lycopersicum
MGNCCGTPGNSSENKKKKNKPNPFALDYGATQASGGDGNKLVVLKDPTGHNIQEKYDLGCELGRGEFGVTYLCTDVDTGDKYACKSISKKKLRTAVDIDDVRREVEIMKHLPKHPNIVTLKDTYEDDNAVHIVMELCEGGELFDRIVARGHYTERAAAVIMKTIVEVVQLHYVSSRNGCSSFNFQLTEFQLTLQMCHMHGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVVDFKRDPWPKVSDNAKDLVKKMLDPDPTRRLTAQQVLEHTWLQNIKKAPNVSLGETVKARLKQFSVMNKLKKRALTIMAEFLSAEEVAGMKDAFDMMDTGKKGKINLGELKNGLQKLGHQIPDVDLQILMEAADVDGDGSLNYGEFVAVSVHLRKMANDEHLHKAFSVFDRDQSGYIEIEELRSALSDEDGGNSEEVINAIMHDVDTDKDGRISYEEFAAMMKAGTDWRKASRQYSRERFNSLSLKLMRDGSIQVGKEEGR

SEQ ID NO: 82 - protein, 558 aa, Solanum lycopersicum; identical to SEQ ID NO: 81

SEQ ID NO: 83 - protein, 557 aa, Solanum lycopersicum
MGNCCGTPGNSSENKKKNKPNPFALDYGATQASGGDGNKLVVLKDPTGHNIQEKYDLGCELGRGEFGVTYLCTDVDTGDKYACKSISKKKLRTAVDIDDVRREVEIMKHLPKHPNIVTLKDTYEDDNAVHIVMELCEGGELFDRIVARGHYTERAAAVIMKTIVEVVQLHYVSSRNGCSSFNFQLTEFQLTLQMCHMHGVMHRDLKPENFLFGNKKEIAPLKAIDFGLSVFFKPGERFNEIVGSPYYMAPEVLKRNYGPEVDVWSAGVILYILLCGVPPFWAETEQGVAQAIIRSVVDFKRDPWPKVSDNAKDLVKKMLDPDPTRRLTAQQVLEHTWLQNIKKAPNVSLGETVKARLKQFSVMNKLKKRALTIMAEFLSAEEVAGMKDAFDMMDTGKKGKINLGELKNGLQKLGHQIPDVDLQILMEAADVDGDGSLNYGEFVAVSVHLRKMANDEHLHKAFSVFDRDQSGYIEIEELRSALSDEDGGNSEEVINAIMHDVDTDKDGRISYEEFAAMMKAGTDWRKASRQYSRERFNSLSLKLMRDGSIQVGKEEGR

SEQ ID NO: 84 - protein, 558 aa, Solanum lycopersicum; identical to SEQ ID NO: 81

SEQ ID NO: 85 - protein, 570 aa, Solanum lycopersicum; identical to SEQ ID NO: 70

SEQ ID NO: 86 - protein, 546 aa, Solanum lycopersicum
MGNCCGTPGNSSENKKKKNKPNPFALDYGATQASGGDGNKLVVLKDPTGHNIQEKYDLGCELGRGEFGVTYLCTDVDTGDKYACKSISKKKLRTAVDIDDVRREVEIMKHLPKHPNIVTLKDTYEDDNAVHIVMELCEGGELFDRIVARGHYTERAAAVIMKTIVEVVQMCHMHGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVVDFKRDPWPKVSDNAKDLVKKMLDPDPTRRLTAQQVLEHTWLQNIKKAPNVSLGETVKARLKQFSVMNKLKKRALTIMAEFLSAEEVAGMKDAFDMMDTGKKGKINLGELKNGLQKLGHQIPDVDLQILMEAADVDGDGSLNYGEFVAVSVHLRKMANDEHLHKAFSVFDRDQSGYIEIEELRSALSDEDGGNSAEVINAIMHDVDTDKLQSSSVTLSITMQDGRISYEEFAAMMKAGTDWRKASRQYSRERFNSLSLKLMRDGSIQVGKEEGR

SEQ ID NO: 87 - protein, 558 aa, Solanum lycopersicum; identical to SEQ ID NO: 81

SEQ ID NO: 88 - protein, 533 aa, Solanum lycopersicum
MGNCCGTPGNSSENKKKKNKPNPFALDYGATQASGGDGNKLVVLKDPTGHNIQEKYDLGCELGRGEFGVTYLCTDVDTGDKYACKSISKKKLRTAVDIDDVRREVEIMKHLPKHPNIVTLKDTYEDDNAVHIVMELCEGGELFDRIVARGHYTERAAAVIMKTIVEVVQMCHMHGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVVDFKRDPWPKVSDNAKDLVKKMLDPDPTRRLTAQQVLEHTWLQNIKKAPNVSLGETVKARLKQFSVMNKLKKRALTIMAEFLSAEEVAGMKDAFDMMDTGKKGKINLGELKNGLQKLGHQIPDVDLQILMEAADVDGDGSLNYGEFVAVSVHLRKMANDEHLHKAFSVFDRDQSGYIEIEELRSALSDEDGGNSAEVINAIMHDVDTDKDGRISYEEFAAMMKAGTDWRKASRQYSRERFNSLSLKLMRDGSIQVGKEEGR

SEQ ID NO: 89 - protein, 570 aa, Solanum lycopersicum; identical to SEQ ID NO: 70

SEQ ID NO: 90 - protein, 546 aa, Solanum lycopersicum; identical to SEQ ID NO: 71

SEQ ID NO: 91 - protein, 570 aa, Solanum lycopersicum; identical to SEQ ID NO: 70

SEQ ID NO: 92 - protein, 558 aa, Solanum lycopersicum; identical to SEQ ID NO: 81

SEQ ID NO: 93 - protein, 570 aa, Solanum lycopersicum; identical to SEQ ID NO: 70

SEQ ID NO: 94 - protein, 558 aa, Solanum lycopersicum; identical to SEQ ID NO: 81

SEQ ID NO: 95 - DNA, 5520 bp, Solanum lycopersicum
  source 1..5520: mol_type = genomic DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcgtggaagtcgttcaggtatagtttttatcagcttcttagttctcagttacctactgcttttattaccttttgcttaggttatcttattatatccattattttcagcatagcttcttcattactgtatttccttttcatattgtttttatatatgtcttacttaggctgagggtccattggaaatagcctctctaccttcacaaggtaggagtaaggctgcgtacacactacccttcccacaccccacttgtgggactatactaagtatgttgttgttttagttctcaattgatgctttttggtttttatgtagtaacttgtggtgtttcttttactgagttcttcaaagcttttgtatgggttttatatcatgacggatgtatcctgtattgttgtttcgggtttttattaaagaagtctggcaatttcaagttaaaaagaaaatcatatcttaaaagagaagcctaatcttgagcaaatcttgagcagatagcaatttcagggtgaaatgtgaatattcttggtgaagttgcatatgaactggtaaccattattcaattcatttctagtgttgatctttgcaatcaatggtttcattctagtgtacgttcatgctgtagcatttaccttgatatcctcatggaatttcctttaccgccaaaattataatgagaagtggttatcttttttaataatggtaaagtatgagaagaggttttcttatgttctcagtctttgctagttctatttctttactcgttttacttaacttttttagtttcttgtgaactgtagtattgctctatatcttctcactcaaattcatgttctaaatcttaaccattctagtgatataaatgaagttaattggtgatataggatatccccctccaaaatctaacaatctttcaactcatcatagtcttgggcagatagctagttagaaccgtaacaaatatttaggaaagtagatcattcgaaatagttatccttgtgaaatattattctttatcttcattattgaccattggttttcaaactctagtactcttaagtggaagattcacttcttgtttctgtggagtattaactatagcggcctacaacagaatttgattgttgcactcaaagttaaacagagttctacagtttagggaaatgataatggaatttaaatttagtttgtatactgaagtaaatatggttgacactatcagaaacaagtgctttttacttcaaatattgatctttatcctcattgcgaaccattgatgtccaaactgttttagatggaagagcaatttgttgttcatttgagtggaatagtcaccaacaacagctaattaactacgtatagcttagagagatgcgaaccaatggtaaatttaagttgtctcctgaagtagttactgattgttcatgataaaggtcattaaagttgaattctctatttagtcagtgtccttttctgcatcgactatatagtggcctgaatgacattacaaaatatttatgagttggtaggaatctttttttcatatgtgaatcatgaatgtgttcacaaatgatcaatcagagagtgattattgtattatgtggtgttgataacttttgaacttttcttcatttggtttgtgtttaactgttatctgaaaggcttggttggtggtactaaagctattggattagcagaaaactttatacatgtttatggtcatgtttcgctctccagtccccgtttttatttgtcaaaggtcagttcatatatttccaatggttttgcagatgtgtcacaggcaaggggtaatgcatcgtgatctcaaacctgagaattttctttttggtaacaagaaagaaacagctccactgaaggctattgactttgggttgtctgttttctttaaacctggtaatggaattggaaaaaaaacagtgagtttaagacttttatcatgctaaacaaaggtttaacatgttgattattttaacaggggaacgctttaatgagatagtgggaagtccttattacatggctcctgaggtcctaaagcgcaattatggaccagaggtcgatgtctggagtgctggagttatactttacattcttctatgcggtgttccacctttctgggcaggtctgcttattttatttcttcgttttttataaccatggaacgaacctcaatgcagatattttcatgtgcaatttttggaacttgataattgattaactaagattgtgaatatgaagtagaacttgattcatgttagaatatgtcgttgaaatattagatgatgaggcaagtgaggtgaaagactgtccctgctttaatcaatgtagatcatgttcaattattttggtttctcttaatggttcttatgggttggactttgctaaataacttgcataaatatgttaacatattcttgatacttcccttaagttcctctgaatatttgcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttggtaatgtttgtggaatattgagatattgtactttattcattcgttgttttactctctctctctctctctctctatctatctctgataagtatttttttccggggctttgactcttgtggtgcagagcatccctggttacataatataaagaaagcaccaaatgtctcattgggtgagactgttaaagcaagactcaagcagttttcagtaatgaacaagctcaagaaaaaagctctgacggtaagcacacacttagtctgcaatcatatttataagctctggattcttcactgtccactaaagatgaattcatatgcttttcctcatagatcgaaatgaaggagagaagattgtgtcttaattatttactgcactgccttgtgctatttaataaaaacttaattctcatcgtaaaaaatgagaggaggttctcaaaattttgatcaagtttcgtttttttatcttgatatatgtactttcttgtattccaaacaggttatagctgagtttttgtctgcggaggaagtcgctggaatgaaggaagcatttgaaatgatggataccggaaagaagggtaagataaacctgaatgaacttaaagatggcttgcagaagcttggccatcaaatccctgatgctgatcttcatattctcatggaagcggtaagcggtctcatgccctgcttcttggctcgatcagatttcttatagctctaccgggggtaacataatatttgaaagttagctgctaaacttttctgcctcaaagagaaaacaggtgcctgagatgatatggccatgtcttacgtagaactgtaaattacatcaatggacattattttaaaatgaccgaaaactacatggaatccatgtgaacttagtaaagaaaatattcaatggaagtaaaggataccaagtagttggattaaggctagattgttggtggtacactgacattagtgtctcaatgtttactaagagtctcagtttgattagagatttgcacgtagatgcagggataaccgtgagattaagaaaattttaagaaattcacggagatgaaaaagatctgtaatactcatatagcttagggccaagcccaactagtttgggatcaaatcgtagttgttgttgataagaatttagcattagacttttgtctgtcaccctactgtcaaaaaattgattgattacaaaatatattttgtcaatataagttgtaacatttttctaccatttcaatgtagaaaaaaggaaaatctgtagcaaaattagcttgacatttcgcttgtcttagcatccctggttcaatgcttattattatccagaaaggaaaaaaaaaacatttctcttgatccttttgcatcttcgacctgaaggatgcttatcttttttattattttatttccttgaacttttgttttatgtttttcttattctgtatcggttttacaggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggtcagtatgtccatttatgtgcacgtacaccatttagtaacttttcgttctttggttcatacttcatagtgcaaatattatgcattgttgctaatgtttacatcttaatgagttctcattttgaagttctcttctgaaccctcgtttgagtttagagctcaaatactccacctagttgtgacttattgtatctctaaagtccgtcatattatataaacgtattatactttgatggcatgcacactttctagcacatatgtgttgctaacgaaactctagtgctgtgacaaatgaaatacatgtatacattgatggcatctgttcttcccatcacatatgtactaccggtcattactctctagtgctgtgacattatgctctccctgttcgcttcatgagttacacggttggttaacctgctgctcgttacatgtctgctacagaattcaagttttgttagctgttttccatctcatctctaacaacatccccactcatttgtattagcttcttatctggagttttgtgagctcagtcttgtatcgtcttgtgtcaaaacgtgtgcaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 96 - DNA, 5526 bp, Solanum lycopersicum
  source 1..5526: mol_type = genomic DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcatgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcgtggaagtcgttcaggtatagtttttatcaacttcttagttctcagttacctactgcttttattaccttttgcttaggttatcttattatatccattattttcagcatagcttcttcattactgtatttccttttcatattgtttttatatatgtcttacttaggctgagggtccattggaaatagcctctctaccttcacaaggtaggagtaaggctgcgtacacactacccttcccacaccccacttgtgggactatactaagtatgttgttgttttagttctcaattgatgctttttggtttttatgtagtaacttgtggtgtttcttttactgagttcttcaaagcttttgtatgggttttatatcatgacggatgtatcctgtattgttgtttcgggtttttattaaagaagtctggcaatttcaagttaaaaagaaaatcatatcttaaaagagaagcctaatcttgagcaaatcttgagcagatagcaatttcagggtgaaatgtgaatattcttggtgaagttgcatatgaactggtaaccattattcaattcatttctagtgttgatctttgcaatcaatggtttcattctagtgtacgttcatgctgtagcatttaccttgatatcctcatggaatttcctttactgccaaaattataatgagaagtggttatcttttttaataatggtaaagtatgagaagaggttttcttatgttctcagtctttgctagttctatttctttactcgttttacttaacttttttagtttcttgtgaactgtagtattgctctatatcttctcactcaaattcatgttctaaatcttaaccattctagtgatataaatgaagttaattggtgatataggatatccccctccaaaatctaacaatctttcaactcatcatagtcttgggcagatagctagttagaaccgtaacaaatatttaggaaagtagatcattcgaaatagttatccttgtgaaatattattctttatcttcattattgaccattggttttcaaactctagtactcttaagtggaagactcacttcttgtttctgtggagtattagctatagcggcctacaacagaatttgattgttgcactcaaagttaaacagagttctacagtttagggaaatgataatggaatttaaatttagtttgtatactgaagtaaatatggttgacactatcagaaacaagtgctttttacttcaaatattgatctttatcctcattgcgaaccattgatgtccaaactgttttagatggaagagcaatttgttgttcatttgagtggaatagtcaccaacaacagctaattaactacgtatagcttagagagatgcgaaccaatggtaaatttaagttgtctcctgaagtagttactgattgttcatgataaaggtcattaaagttgaattctctatttagtcagtgtccttttctgcatcaactatatagtggcctgaatgacattacaaaatatttatgagttggtaggaatctttttttcatatgtgaatcatgaatgtgttcacaaatgatcaatcagagagtgattattgtattatgtggtgttgataacttttgaacttttcttcatttggtttgtgtttaactgttatctgaaaggcttggttggtggtactaaagctattggattagcagaaaactttatacatgtttatggtcatgtttcgctctccagtccccgtttttatttgtcaaaggtcagttcatatatttccaatggttttgcagatgtgtcacaggcaaggggtaatgcatcgtgatctcaaacctgagaattttctttttggtaacaagaaagaaacagctccactgaaggctattgactttgggttgtctgttttctttaaacctggtaatggaattggaaaaaaaacagtgagtttaagacttttatcatgctaaacaaaggtttaacatgttgattattttaacaggggaacgctttaatgagatagtgggaagtccttattacatggctcctgaggtcctaaagcgcaattatggaccagaggtcgatgtctggagtgctggagttatactttacattcttctatgcggtgttccacctttctgggcaggtctgcttattttatttcttcgttttttataaccatggaacgaacctcaatgcagatattttcatgtgcaatttttggaacttgataattgattaactaagattgtgaatatgaagtagaacttgattcatgttagaatatgttgttgaaatattagatgatgaggcaagtgaggtgaaagactgtccctgctttaatcaatgtagatcatgttcaattattttggtttctcttaatggttcttatgggttggactttgctaaataacttgcataaatatgttaacatattcttgatacttcccttaagttcctctgaatatttgcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttggtaatgtttgtggaatattgagatattgtactttattcattcgttgttttactctctctctctctctctctctctctctatctatctctgataagtatttttttccggggctttgactcttgtggtgcagagcatccctggttacataatataaagaaagcaccaaacgtctcattgggtgagactgttaaagcaagactcaagcagttttcagtaatgaacaagctcaagaaaaaagctctgacggtaagcacacacttagtctgcaatcatatttataagctctggattcttcactgtccactaaagatgaattcatatgcttttcctcatagatcgaaatgaaggagagaaggttgtgtcttaattatttactgcactgccttgtgctatttaataaaaacttaattctcatcgtaaaaaatgagaggcggttctcaaaattttgatcaagtttcgtttttttatcttgatatatgtactttcttgtattccaaacaggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggtaagcggtctcatgccctgcttcttggctcgatcagatttcttatagctctaccgggggtaacataatatttgaaagttagctgctaaacttttctgcctcaaagagaaaacaggtgcctgagatgatatggccatgtcttacgtagaactgtaaattacatcaatggacattattttaaaatgaccgaaaactacatggaatccatgtgaacttagtaaagaaaatattcaatggaagtaaaggataccaagtagttggattaaggctagattgttggtggtacactgacattagtgtctcaatgtttactaagagtctcagtttgattagagatttgcacgtagatgcagggataaccgtgagattaagaaaattttaagaaattcacggagatgaaaaagatctgtaatactcatatagcttagggccaagcccaactagtttgggatcaaatcgtagttgttgttgataagaatttagcattagacttttgtctgtcaccctactgtcaaaaaattgattgattacaaaatatattttgtcaatataagttgtaacatttttctaccatttcaatgtagaaaaaaggaaaatctgtagcaaaattagcttgacatttcgcttgtcttatcatccctggttcaatgcttattattatccagaaaggaaaaaaaaaacatttctcttgatccttttgcatcttcgacctgaaggatgcttatcttttttattattttatttccttgaacttttgttttatgtttttcttattctgtatcggttttacaggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggtcagtatgtccatttatgtgcacgtacaccatttagtaacttttcgttctttggttcatacttcatagtgcaaatattatgcattgttgctaatgtttacatcttaatgagttctcattttgaagttctcttctgaaccctcgtttgagtttagagctcaaatactccacctagttgtgacttattgtatctctaaagtccgtcatattatataaacgtattatactttgatggcatgcacactttctagcacatatgtgttgctaacgaaactctagtgctgtgacaaatgaaatacatgtatacattgatggcatctgttcttcccatcacatatgtactaccggtcattactctctagtgctgtgacattatgctctccctgttcgcttcatgagttacacggttggttaacctgctgctcgttacatgtctgctacagaattcaagttttgttagctgttttccatctcatctctaacaacatccccactcatttgtattagcttcttatctggagttttgtgagctcagtcttgtatcgtcttgtgtcaaaacgtgtgcaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 97 - DNA, 5520 bp, Solanum lycopersicum
  source 1..5520: mol_type = genomic DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcgtggaagtcgttcaggtatagtttttatcagcttcttagttctcagttacctactgcttttattaccttttgcttaggttatcttattatatccattattttcagcatagcttcttcattactgtatttccttttcatattgtttttatatatgtcttacttaggctgagggtccattggaaatagcctctctaccttcacaaggtaggagtaaggctgcgtacacactacccttcccacaccccacttgtgggactatactaagtatgttgttgttttagttcacaattgatgctttttggtttttatgtagtaacttctggtgtttcttttactgagttcttcaaagcttttgtatgggttttatatcatgacggatgtatcctgtattgttgtttcgggtttttattaaagaagtctggcaatttcaagttaaaaagaaaatcatatcttaaaagagaagcctaatcttgagcagatagcaatttcagggtgaaatgtgaatattcttggtgaagttgcatatgaacatatgaactggctaaccattattcaattcatttctagtgttgatctttgcaatcaatggtttcattctagtgtacgttcatgctgtagcatttaccttgatatcctcatggaatttcctttaccgccaaaattataatgagaagtggttatcttttttaataatggtaaagtatgagaagaggttttcttatgttctcagtctttgctagttctatttctttactcgttttacttaacttttttagtttcttgtgaactgtagtattgctctatatcttctcactcaaattcatgttctaaatcttaaccattctagtgatataaatgaagttaattggtgatataggatatccccctccaaaatctaacaatctttcaactcatcatagtcttgggcagatagctagttagaaccgtaacaaatatttaggaaagtagatcattcgaaatagttatccttgtgaaatattattctttatcttcattattgaccattggttttcaaactctagtactcttaagtggaagactcacttcttgtttctgtggagtattagctatagcggcctacaacagaatttgattgttgcactcaaagttaaacagagttctacagtttagggaaatgataatggaatttaaatttagtttgtatactgaagtaaatatggttgacactatcagaaacaagtgctttttacttcaaatattgatctttatcctcattgcgaaccattgatgtccaaactgttttagatggaagagcaatttgttgttcatttgagtggaatagtcaccaacaacagctaattaactacgtatagcttagagagatgcgaaccaatggtaaatttaagttgtctcctgaagtagttactgattgttcatgataaaggtcattaaagttgaattctctatttagtcagcgtccttttctgcatcgactatatagtggcctgaatgacattacaaaatatttatgagttggtaggaatctttttttcatatgtgaatcatgaatgtgttcacaaatgatcaatcagagagtgattattgtattatgtggtgttgataacttttgaacttttcttcatttggtttgtgtttaactgttatctgaaaggcttggttggtggtactaaagctattggattagcagaaaactttatacatgtttatggtcatgtttcgctctccagtctccgtttttatttgtcaaaggtcagttcatatatttccaatggttttgcagatgtgtcacaggcaaggggtaatgcatcgtgatctcaaacctgagaattttctttttggtaacaagaaagaaacagctccactgaaggctattgactttgggttgtctgttttctttaaacctggtaatggaattggaaaagaaacagtgagtttaagacttttatcatgctaaacaaaggtttaacattttgattattttaacaggggaacgctttaatgagatagtgggaagtccttattacatggctcctgaggtcctaaagcgcaattatggaccagaggtcgatgtctggagtgctggagttatactttacattcttctatgcggtgttccacctttctgggcaggtctgcttattttatttcttcgttttttataaccatggaacgaacctcaatgcagatattttcatgtgcaatttttggaacttgataattgattaactaagattgtgaatatgaagtagaacttgattcatgttagaatatgtcgttgaaatattagatgatgaggcaagtgaggtgaaagactgtccctgctttaatcaatgtagatcatgttcaattattttggtttctcttaatggttcttatgggttggactttgctaaataacttgcataaatatgttaacatattcttgatacttcccttaagttcctctgaatatttgcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttggtaatgtttgtggaatattgagatattgtactttattcattcgttgttttactctctctctctctctctctctctatctatctctgataagtattttttttccggggctttgactcttgtggtgcagagcatccctggttacataatataaagaaagcaccaaacgtctcattgggtgagactgttaaagcaagactcaagcagttttcagtaatgaacaagctcaagaaaaaagctctgacggtaagcacacacttagtctgcaatcatatttataagctctggtttcttcactgtccactaaagatgaattcatatgcttttcctcatagatcgaaatgaaggagagaaggttgtgtcttaattatttactgcactgccttgtgctatttaataaaaacttaattctcatcgtaaaaaatgagaggcggttctcaaaattttgatcaagtttcgtttttttatcttgatatatgtactttcttgtattccaaacaggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggtaagcggtctcatgccctgcttcttggctcgatcagatttcttatagctctaccgggggtaacataatatttgaaagttagctgctaaacttttctgcctcaaagagaaaacaggtgcctgagatgatatggccatgtcttacgtagaactgtaaattacatcaatggacattattttaaaatgaccgaaaactacatggaatccatgtgaacttagtaaagaaaatattcaatggaagtaaaggataccaagtagttggattaaggctagattgttggtggtacactgacattagtgtctcaatgtttactaagagtctcagtttgattagagatttgcacgtagatgcagggataaccgtgagattaagaaaattttaagaaattcacggagatgaaaaagatctgtaatactcatatagcttagggccaagcccaactagtttgggatcaaatcgtagttgttgttgataagaatttagcattagacttttgtctgtcaccctactgtcaaaaaattgattgattacaaaatatattttgtcaatataagttgtaacatttttctaccatttcaatgtagaaaaaaggaaaatctgtagcaaaattagcttgacatttcgcttgtcttagcatccctggttcaatgcttattattatccagaaaggaaaaaaaaacatttctcttgatccttttgcatcttcgacctgaaggatgcttatcttttttattattttatttccttgaacttttgttttatgtttttcttattctgtatcggttttacaggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggtcagtatgtccatttatgtgcacgtacaccatttagtaacttttcgttctttggttcatacttcatagtgcaaatattatgcatcgatgctaatgtttacatcttaatgagttctcattttgaagttcccttctgaaccctcgtttgagtttagagctcaaatactccacctagttgtgacttattgtatctctaaagtccgtcatgttatataaacgtattatactttgatggcatgcacactttctagcacatatgtgttgctaacgaaactctagtgctgtgacaaatgaaatacatgtatacattgatggcatctgttcttcccatcacatatgtactaccggtcattactctctagtgctgtgacattatgctctccctgttcgcttcatgatttacacggttggttaacctgctgctcgttacatgtctgctacagaattcaagttttgttagctgttttccatctcatctctaacaacatccccactcatttgtattagcttcttatctggagttttgtgagctcagtcttgtatcgtcttgtgtcaaaacgtgtgcaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 98 - DNA, 5517 bp, Solanum lycopersicum
  source 1..5517: mol_type = genomic DNA
atgggtaattgctgtgtaaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcgtggaagtcgttcaggtatagtttttatcagcttcttagttctcagttacctactgcttttattaccttttgcttaggttatcttattatatccattattttcagcatagcttcttcattactgtatttccttttcatattgtttttatatatgtcttacttaggctgagggtccattggaaatagcctctctaccttcacaaggtaggagtaaggctgcgtacacactacccttcccacaccccacttgtgggactatactaagtatgttgttgttttagttcacaattgatgctttttggtttttatgtagtaacttctggtgtttcttttactgagttcttcaaagcttttgtatgggttttatatcatgacggatgtatcctgtattgttgtttcgggtttttattaaagaagtctggcaatttcaagttaaaaagaaaatcatatcttaaaagagaagcctaatcttgagcagatagcaatttcagggtgaaatgtgaatattcttggtgaagttgcatatgaacatatgaactggctaaccattattcaattcatttctagtgttgatctttgcaatcaatggtttcattctagtgtacgttcatgctgtagcatttaccttgatatcctcatggaatttcctttaccgccaaaattataatgagaagtggttatcttttttaataatggtaaagtatgagaagaggttttcttatgttctcagtctttgctagttctatttctttactcgttttacttaacttttttagtttcttgtgaactgtagtattgctctatatcttctcactcaaattcatgttctaaatcttaaccattctagtgatataaatgaagttaattggtgatataggatatccccctccaaaatctaacaatctttcaactcatcatagtcttgggcagatagctagttagaaccgtaacaaatatttaggaaagtagatcattcgaaatagttatccttgtgaaatattattctttatcttcattattgaccattggttttcaaactctagtactcttaagtggaagactcacttcttgtttctgtggagtattagctatagcggcctacaacagaatttgattgttgcactcaaagttaaacagagttctacagtttagggaaatgataatggaatttaaatttagtttgtatactgaagtaaatatggttgacactatcagaaacaagtgctttttacttcaaatattgatctttatcctcattgcgaaccattgatgtccaaactgttttagatggaagagcaatttgttgttcatttgagtggaatagtcaccaacaacagctaattaactacgtatagcttagagagatgcgaaccaatggtaaatttaagttgtctcctgaagtagttactgattgttcatgataaaggtcattaaagttgaattctctatttagtcagcgtccttttctgcatcgactatatagtggcctgaatgacattacaaaatatttatgagttggtaggaatctttttttcatatgtgaatcatgaatgtgttcacaaatgatcaatcagagagtgattattgtattatgtggtgttgataacttttgaacttttcttcatttggtttgtgtttaactgttatctgaaaggcttggttggtggtactaaagctattggattagcagaaaactttatacatgtttatggtcatgtttcgctctccagtctccgtttttatttgtcaaaggtcagttcatatatttccaatggttttgcagatgtgtcacaggcaaggggtaatgcatcgtgatctcaaacctgagaattttctttttggtaacaagaaagaaacagctccactgaaggctattgactttgggttgtctgttttctttaaacctggtaatggaattggaaaagaaacagtgagtttaagacttttatcatgctaaacaaaggtttaacattttgattattttaacaggggaacgctttaatgagatagtgggaagtccttattacatggctcctgaggtcctaaagcgcaattatggaccagaggtcgatgtctggagtgctggagttatactttacattcttctatgcggtgttccacctttctgggcaggtctgcttattttatttcttcgttttttataaccatggaacgaacctcaatgcagatattttcatgtgcaatttttggaacttgataattgattaactaagattgtgaatatgaagtagaacttgattcatgttagaatatgtcgttgaaatattagatgatgaggcaagtgaggtgaaagactgtccctgctttaatcaatgtagatcatgttcaattattttggtttctcttaatggttcttatgggttggactttgctaaataacttgcataaatatgttaacatattcttgatacttcccttaagttcctctgaatatttgcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttggtaatgtttgtggaatattgagatattgtactttattcattcgttgttttactctctctctctctctctatctatctatctctgataagtatttttttccggggctttgactcttgtggtgcagagcatccctggttacataatataaagaaagcaccaaacgtctcattgggtgagactgttaaagcaagactcaagcagttttcagtaatgaacaagctcaagaaaaaagctctgacggtaagcacacacttagtctgcaatcatatttataagctctggtttcttcactgtccactaaagatgaattcatatgcttttcctcatagatcgaaatgaaggagagaaggttgtgtcttaattatttactgcactgccttgtgctatttaataaaaacttaattctcatcgtaaaaaatgagaggcggttctcaaaattttgatcaagtttcgtttttttatcttgatatatgtactttcttgtattccaaacaggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggtaagcggtctcatgccctgcttcttggctcgatcagatttcttatagctctaccgggggtaacataatatttgaaagttagctgctaaacttttctgcctcaaagagaaaacaggtgcctgagatgatatggccatgtcttacgtagaactgtaaattacatcaatggacattattttaaaatgaccgaaaactacatggaatccatgtgaacttagtaaagaaaatattcaatggaagtaaaggataccaagtagttggattaaggctagattgttggtggtacactgacattagtgtctcaatgtttactaagagtctcagtttgattagagatttgcacgtagatgcagggataaccgtgagattaagaaaattttaagaaattcacggagatgaaaaagatctgtaatactcatatagcttagggccaagcccaactagtttgggatcaaatcgtagttgttgttgataagaatttagcattagacttttgtctgtcaccctactgtcaaaaaattgattgattacaaaatatattttgtcaatataagttgtaacatttttctaccatttcaatgtagaaaaaaggaaaatctgtagcaaaattagcttgacatttcgcttgtcttagcatccctggttcaatgcttattattatccagaaaggaaaaaaaaacatttctcttgatccttttgcatcttcgacctgaaggatgcttatcttttttattattttatttccttgaacttttgttttatgtttttcttattctgtatcggttttacaggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggtcagtatgtccatttatgtgcacgtacaccatttagtaacttttcgttctttggttcatacttcatagtgcaaatattatgcatcgatgctaatgtttacatcttaatgagttctcattttgaagttcccttctgaaccctcgtttgagtttagagctcaaatactccacctagttgtgacttattgtatctctaaagtccgtcatgttatataaacgtattatactttgatggcatgcacactttctagcacatatgtgttgctaacgaaactctagtgctgtgacaaatgaaatacatgtatacattgatggcatctgttcttcccatcacatatgtactaccggtcattactctctagtgctgtgacattatgctctccctgttcgcttcatgatttacacggttggttaacctgctgctcgttacatgtctgctacagaattcaagttttgttagctgttttccatctcatctctaacaacatccccactcatttgtattagcttcttatctggagttttgtgagctcagtcttgtatcgtcttgtgtcaaaacgtgtgcaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 99 - DNA, 5525 bp, Solanum lycopersicum
  source 1..5525: mol_type = genomic DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcatgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcgtggaagtcgttcaggtatagtttttatcaacttcttagttctcagttacctactgcttttattaccttttgcttaggttatcttattatatccattattttcagcatagcttcttcattactgtatttccttttcatattgtttttatatatgtcttacttaggctgagggtccattggaaatagcctctctaccttcacaaggtaggagtaaggctgcgtacacactacccttcccacaccccacttgtgggactatactaagtatgttgttgttttagttctcaattgatgctttttggtttttatgtagtaacttgtggtgtttcttttactgagttcttcaaagcttttgtatgggttttatatcatgacggatgtatcctgtattgttgtttcgggtttttattaaagaagtctggcaatttcaagttaaaaagaaaatcatatcttaaaagagaagcctaatcttgagcaaatcttgagcagatagcaatttcagggtgaaatgtgaatattcttggtgaagttgcatatgaactggtaaccattattcaattcatttctagtgttgatctttgcaatcaatggtttcattctagtgtacgttcatgctgtagcatttaccttgatatcctcatggaatttcctttactgccaaaattataatgagaagtggttatcttttttaataatggtaaagtatgagaagaggttttcttatgttctcagtctttgctagttctatttctttactcgttttacttaacttttttagtttcttgtgaactgtagtattgctctatatcttctcactcaaattcatgttctaaatcttaaccattctagtgatataaatgaagttaattggtgatataggatatccccctccaaaatctaacaatctttcaactcatcatagtcttgggcagatagctagttagaaccgtaacaaatatttaggaaagtagatcattcgaaatagttatccttgtgaaatattattctttatcttcattattgaccattggttttcaaactctagtactcttaagtggaagactcacttcttgtttctgtggagtattagctatagcggcctacaacagaatttgattgttgcactcaaagttaaacagagttctacagtttagggaaatgataatggaatttaaatttagtttgtatactgaagtaaatatggttgacactatcagaaacaagtgctttttacttcaaatattgatctttatcctcattgcgaaccattgatgtccaaactgttttagatggaagagcaatttgttgttcatttgagtggaatagtcaccaacaacagctaattaactacgtatagcttagagagatgcgaaccaatggtaaatttaagttgtctcctgaagtagttactgattgttcatgataaaggtcattaaagttgaattctctatttagtcagtgtccttttctgcatcgactatatagtggcctgaatgacattacaaaatatttatgagttggtaggaatctttttttcatatgtgaatcatgaatgtgttcacaaatgatcaatcagagagtgattattgtattatgtggtgttgataacttttgaacttttcttcatttggtttgtgtttaactgttatctgaaaggcttggttggtggtactaaagctattggattagcagaaaactttatacatgtttatggtcatgtttcgctctccagtccccgtttttatttgtcaaaggtcagttcatatatttccaatggttttgcagatgtgtcacaggcaaggggtaatgcatcgtgatctcaaacctgagaattttctttttggtaacaagaaagaaacagctccactgaaggctattgactttgggttgtctgttttctttaaacctggtaatggaattggaaaaaaaacagtgagtttaagacttttatcatgctaaacaaaggtttaacatgttgattattttaacaggggaacgctttaatgagatagtgggaagtccttattacatggctcctgaggtcctaaagcgcaattatggaccagaggtcgatgtctggagtgctggagttatactttacattcttctatgcggtgttccacctttctgggcaggtctgcttattttatttcttcgttttttataaccatggaacgaacctcaatgcagatattttcatgtgcaatttttggaacttgataattgattaactaagattgtgaatatgaagtagaacttgattcatgttagaatatgttgttgaaatattagatgatgaggcaagtgaggtgaaagactgtccctgctttaatcaatgtagatcatgttcaattattttggtttctcttaatggttcttatgggttggactttgctaaataacttgcataaatatgttaacatattcttgatacttcccttaagttcctctgaatatttgcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttggtaatgtttgtggaatattgagatattgtactttattcattcgttgttttactctctctctctctctctctctctctctatctatctctgataagtatttttttccggggctttgactcttgtggtgcagagcatccctggttacataatataaagaaagcaccaaacgtctcattgggtgagactgttaaagcaagactcaagcagttttcagtaatgaacaagctcaagaaaaaagctctgacggtaagcacacacttagtctgcaatcatatttataagctctggattcttcactgtccactaaagatgaattcatatgcttttcctcatagatcgaaatgaaggagagaaggttgtgtcttaattatttactgcactgccttgtgctatttaataaaaacttaattctcatcgtaaaaaatgagaggcggttctcaaaattttgatcaagtttcgtttttttatcttgatatatgtactttcttgtattccaaacaggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggtaagcggtctcatgccctgcttcttggctcgatcagatttcttatagctctaccgggggtaacataatatttgaaagttagctgctaaacttttctgcctcaaagagaaaacaggtgcctgagatgatatggccatgtcttacgtagaactgtaaattacatcaatggacattattttaaaatgaccgaaaactacatggaatccatgtgaacttagtaaagaaaatattcaatggaagtaaaggataccaagtagttggattaaggctagattgttggtggtacactgacattagtgtctcaatgtttactaagagtctcagtttgattagagatttgcacgtagatgcagggataaccgtgagattaagaaaattttaagaaattcacggagatgaaaaagatctgtaatactcatatagcttagggccaagcccaactagtttgggatcaaatcgtagttgttgttgataagaatttagcattagacttttgtctgtcaccctactgtcaaaaaattgattgattacaaaatatattttgtcaatataagttgtaacatttttctaccatttcaatgtagaaaaaaggaaaatctgtagcaaaattagcttgacatttcgcttgtcttagcatccctggttcaatgcttattattatccagaaaggaaaaaaaaacatttctcttgatccttttgcatcttcgacctgaaggatgcttatcttttttattattttatttccttgaacttttgttttatgtttttcttattctgtatcggttttacaggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggtcagtatgtccatttatgtgcacgtacaccatttagtaacttttcgttctttggttcatacttcatagtgcaaatattatgcattgttgctaatgtttacatcttaatgagttctcattttgaagttctcttctgaaccctcgtttgagtttagagctcaaatactccacctagttgtgacttattgtatctctaaagtccgtcatattatataaacgtattatactttgatggcatgcacactttctagcacatatgtgttgctaacgaaactctagtgctgtgacaaatgaaatacatgtatacattgatggcatctgttcttcccatcacatatgtactaccggtcattactctctagtgctgtgacattatgctctccctgttcgcttcatgagttacacggttggttaacctgctgctcgttacatgtctgctacagaattcaagttttgttagctgttttccatctcatctctaacaacatccccactcatttgtattagcttcttatctggagttttgtgagctcagtcttgtatcgtcttgtgtcaaaacgtgtgcaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 100 - DNA, 5517 bp, Solanum lycopersicum; identical to SEQ ID NO: 98
  source 1..5517: mol_type = genomic DNA

SEQ ID NO: 101 - DNA, 5516 bp, Solanum lycopersicum
  source 1..5516: mol_type = genomic DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcgtggaagtcgttcaggtatagtttttatcagcttcttagttctcagttacctactgcttttattaccttttgcttaggttatcttattatatccattattttcagcatagcttcttcattactgtatttccttttcatattgtttttatatatgtcttacttaggctgagggtccattggaaatagcctctctaccttcacaaggtaggagtaaggctgcgtacacactacccttcccacaccccacttgtgggactatactaagtatgttgttgttttagttctcaattgatgctttttggtttttatgtagtaacttgtggtgtttcttttactgagttcttcaaagcttttgtatgggttttatatcatgacggatgtatcctgtattgttgtttcgggtttttattaaagaagtctggcaatttcaagttaaaaagaaaatcatatcttaaaagagaagcctaatcttgagcaaatcttgagcagatagcaatttcagggtgaaatgtgaatattcttggtgaagttgcatatgaactggtaaccattattcaattcatttctagtgttgatctttgcaatcaatggtttcattctagtgtacgttcatgctgtagcatttaccttgatatcctcatggaatttcctttaccgccaaaattataatgagaagtggttatcttttttaataatggtaaagtatgagaagaggttttcttatgttctcagtctttgctagttctatttctttactcgttttacttaacttttttagtttcttgtgaactgtagtattgctctatatcttctcactcaaattcatgttctaaatcttaaccattctagtgatataaatgaagttaattggtgatataggatatccccctccaaaatctaacaatctttcaactcatcatagtcttgggcagatagctagttagaaccgtaacaaatatttaggaaagtagatcattcgaaatagttatccttgtgaaatattattctttatcttcattattgaccattggttttcaaactctagtactcttaagtggaagattcacttcttgtttctgtggagtattaactatagcggcctacaacagaatttgattgttgcactcaaagttaaacagagttctacagtttagggaaatgataatggaatttaaatttagtttgtatactgaagtaaatatggttgacactatcagaaacaagtgctttttacttcaaatattgatctttatcctcattgcgaaccattgatgtccaaactgttttagatggaagagcaatttgttgttcatttgagtggaatagtcaccaacaacagctaattaactacgtatagcttagagagatgcgaaccaatggtaaatttaagttgtctcctgaagtagttactgattgttcatgataaaggtcattaaagttgaattctctatttagtcagtgtccttttctgcatcgactatatagtggcctgaatgacattacaaaatatttatgagttggtaggaatctttttttcatatgtgaatcatgaatgtgttcacaaatgatcaatcagagagtgattattgtattatgtggtgttgataacttttgaacttttcttcatttggtttgtgtttaactgttatctgaaaggcttggttggtggtactaaagctattggattagcagaaaactttatacatgtttatggtcatgtttcgctctccagtccccgtttttatttgtcaaaggtcagttcatatatttccaatggttttgcagatgtgtcacaggcaaggggtaatgcatcgtgatctcaaacctgagaattttctttttggtaacaagaaagaaacagctccactgaaggctattgactttgggttgtctgttttctttaaacctggtaatggaattggaaaaaaaacagtgagtttaagacttttatcatgctaaacaaaggtttaacatgttgattattttaacaggggaacgctttaatgagatagtgggaagtccttattacatggctcctgaggtcctaaagcgcaattatggaccagaggtcgatgtctggagtgctggagttatactttacattcttctatgcggtgttccacctttctgggcaggtctgcttattttatttcttcgttttttataaccatggaacgaacctcaatgcagatattttcatgtgcaatttttggaacttgataattgattaactaagattgtgaatatgaagtagaacttgattcatgttagaatatgtcgttgaaatattagatgatgaggcaagtgaggtgaaagactgtccctgctttaatcaatgtagatcatgttcaattattttggtttctcttaatggttcttatgggttggactttgctaaataacttgcataaatatgttaacatattcttgatacttcccttaagttcctctgaatatttgcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttggtaatgtttgtggaatattgagatattgtactttattcattcgttgttttactctctctctctctctctatctatctctgataagtatttttttccggggctttgactcttgtggtgcagagcatccctggttacataatataaagaaagcaccaaatgtctcattgggtgagactgttaaagcaagactcaagcagttttcagtaatgaacaagctcaagaaaaaagctctgacggtaagcacacacttagtctgcaatcatatttataagctctggattcttcactgtccactaaagatgaattcatatgcttttcctcatagatcgaaatgaaggagagaagattgtgtcttaattatttactgcactgccttgtgctatttaataaaaacttaattctcatcgtaaaaaatgagaggaggttctcaaaattttgatcaagtttcgtttttttatcttgatatatgtactttcttgtattccaaacaggttatagctgagtttttgtctgcggaggaagtcgctggaatgaaggaagcatttgaaatgatggataccggaaagaagggtaagataaacctgaatgaacttaaagatggcttgcagaagcttggccatcaaatccctgatgctgatcttcatattctcatggaagcggtaagcggtctcatgccctgcttcttggctcgatcagatttcttatagctctaccgggggtaacataatatttgaaagttagctgctaaacttttctgcctcaaagagaaaacaggtgcctgagatgatatggccatgtcttacgtagaactgtaaattacatcaatggacattattttaaaatgaccgaaaactacatggaatccatgtgaacttagtaaagaaaatattcaatggaagtaaaggataccaagtagttggattaaggctagattgttggtggtacactgacattagtgtctcaatgtttactaagagtctcagtttgattagagatttgcacgtagatgcagggataaccgtgagattaagaaaattttaagaaattcacggagatgaaaaagatctgtaatactcatatagcttagggccaagcccaactagtttgggatcaaatcgtagttgttgttgataagaatttagcattagacttttgtctgtcaccctactgtcaaaaaattgattgattacaaaatatattttgtcaatataagttgtaacatttttctaccatttcaatgtagaaaaaaggaaaatctgtagcaaaattagcttgacatttcgcttgtcttagcatccctggttcaatgcttattattatccagaaaggaaaaaaaaaacatttctcttgatccttttgcatcttcgacctgaaggatgcttatcttttttattattttatttccttgaacttttgttttatgtttttcttattctgtatcggttttacaggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggtcagtatgtccatttatgtgcacgtacaccatttagtaacttttcgttctttggttcatacttcatagtgcaaatattatgcattgttgctaatgtttacatcttaatgagttctcattttgaagttctcttctgaaccctcgtttgagtttagagctcaaatactccacctagttgtgacttattgtatctctaaagtccgtcatattatataaacgtattatactttgatggcatgcacactttctagcacatatgtgttgctaacgaaactctagtgctgtgacaaatgaaatacatgtatacattgatggcatctgttcttcccatcacatatgtactaccggtcattactctctagtgctgtgacattatgctctccctgttcgcttcatgagttacacggttggttaacctgctgctcgttacatgtctgctacagaattcaagttttgttagctgttttccatctcatctctaacaacatccccactcatttgtattagcttcttatctggagttttgtgagctcagtcttgtatcgtcttgtgtcaaaacgtgtgcaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 102 - DNA, 5501 bp, Solanum lycopersicum
  source 1..5501: mol_type = genomic DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagggcagctgctggaattttgaagactgtcgtggaagttgttcaggtatagtttttatcagcttcttagttcacagttactactgtttttattaccttttgcttaggttatcttattatatccattattttcagcatagcttcttcattactgtatttccttttcatattgtttttatatatgtcttacttaggctgagggtccattggaaatagcctctctaccttcacaaggtaggagtaaggctgcgtacacactacccttcccacaccccacttgtgggactatactaagtatgttgttgttttagttcacaattgatgctttttgttttttatgtagtaacttgtggtgtttcttttactgagttcttcaaagcttttgtatgggttttatatcatgacggatgtatcctgtattgttgtttcgggtttttattaaagaagtctggcaatttcaagttaaaaagaaaatcatatcttaaaagagaagcctaatcttgagcagatagcaatttcagggtgaaatgtgaatattcttggtgaagttgcatatgaactggctaaccattattcaattcatttctagtgttgatctttgcaatcaatggtttcattctagtgtacgttcatgctgtagcatttaccttgatatcctcatggaatttcctttaccgccaaaattataatgagaagtggttatcttttttaataatggtaaagtatgagaagaggttttcttatgttctcagtctttgctagttctatttctttactcgttttacttaacttttttagtttcttgtgaactgtagtattgctctatatcttctcactcaaattcatgttctaaatcttaaccattctagtgatataaatgaagttaattggtgatataggatatccccctccaaaatctaacaatctttcaactcatcatagtcttgggcagatagctagttagaaccgtaacaaatatttaggaaagtagatcattcgaaatagttatccttgtgaaatattattctttatcttcattattgaccattggttttcaaactctagtactcttaagtggaagactcacttcttgtttctgtggagtattagctatagcggcctacaacagaatttgattgttgcactcaaagttaaacagagttctacagtttagggaaatgataatggaatttaaatttagtttgtatactgaagtaaatatggttgacactatcagaaacaagtgctttttacttcaaatattgatctttatcctcattgcgaaccattgatgtccaaactgttttagatggaagagcaatttgttgttcatttgagtggaatagtcaccaacaacagctaattaactacgtatagcttagagagatgcgaaccaatggtaaatttaagttgtctcctgaagtagttactgattgttcacgataaaggtcattaaagttgaattctctatttagtcagcgtccttttctgcatcgactatatagtggcctgaatgacattacaaaatatttatgagttggtaggaatctttttttcatatgtgaatcatgaatgtgttcacaaatgatcaatcagagagtgattattgtattatgtggtgttgataacttttgaacttttcttcatttggtttgtgtttaactgttatctgaaaggcttggttggtggtactaaagctattggattagcagaaaactttatacatgtttatggtcatgtttcgctctccagtctccgtttttatttgtcaaaggtcagttcatatatttccaatggttttgcagatgtgtcacaggcaaggggtaatgcatcgtgatctcaaacctgagaattttctttttggtaacaagaaagaaacagctccactgaaggctattgactttgggttgtctgttttctttaaacctggtaatggaattggaaaaaaaacagtgagtttaagacttttatcatgctaaacaaaggtttaacattttgattattttaacaggggaacgctttaatgagatagtgggaagtccttattacatggctcctgaggtcctaaagcgcaattatggaccagaggtcgatgtctggagtgctggagttatactttacattcttctatgcggtgttccacctttctgggcaggtctgcttattttatttcttcgttttttataaccatggaacgaacctcaatgcagatattttcatgtgcaatttttggaacttgataattgattaactaagattgtgaatatgaagtagaacttgattcatgttagaatatgtcgttgaaatattagatgatgaggcaagtgaggtgaaagactgtccctgctttaatcaatgtagatcatgttcaattattttggtttctcttaatggttcttatgggttggactttgctaaataacttgcataaatatgttaacatattcttgatacttcccttaagttcctctgaatatttgcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttggtaatgtttgtggaatattgagatattgtacttcattcattcgttgttttactctctctctctctctctctatctatgataagtatttttttccggggctttgactcttgtggtgcagagcatccctggttacataatataaagaaagcaccaaacgtctcattgggtgagactgttaaagcaagactcaagcagttttcagtaatgaacaagctcaagaaaaaagctctgacggtaagcacacacttagtctgcaatcatatttataagctctggattcttcactgtccactaaagatgaattcatatgcttttcctcatagattgaaatgaaggagagaaggttgtgtcttaattatttactgcactgccttgtgctatttaataaaaacttaattctcatcgtaaaaaatgagagacggttctcaaaattttgatcaggtttcgtttttttatcttgatatatgtactttcttgtattccaaacaggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcagattctcatggaagcggtaagcggtctcatgccctgcttcttggctcgatcagatttcttatagctctaccgggggtaacataatatttgaaagttagctgctaaacttttctgcctcaaagagaaaacaggtgcctgagatgatatggccatgtcttacgtagaactgtaaattacatcaatggacattattttaaaatgaccgaaaactacatggaatccatgtgaacttagtaaagaaaatactcaatggaaggaagtaaaggataccaagtagttggattaaggctagattgttggtggtacactgacattagtgactcaatgtttactaagactctcagtttgattagagatttgcacgtagatgcagggataaccgtgagattaagaaaattttaagaaattcgcggagatgaaatagatctgtaatactcatatagcttagggccaagcccaactagtttgggatcaaatcgtagttgttgttgatgagaatttagcattagacttttgtctgtcaccctactgtcaaaaaattgattgattacaaaatatattttgtcaatataagttgtaacatttttctaccatttcaatgtagaaaaaaggaaaatgtagcaaaattagcttgacatttcgcttgtcttagcatccctggttcaatgcttattattatccagaaaggaaaaaaaaaacatttctcttgatccttttgcatcttcgacctgaaggatgcttatcttttttattattttatttccttgaacttttgttttatgtttttcttattctgtatcggttttacaggctgacgttgatggagatggaagtttaaattatccagagtttgttgctgtatctattcatcttagaaagatggccaatgatgaacacctgcacaaagcattttcattttttgacaaaaaccaaagtggtttcatagaaatcgaagagcttcgtagtgctttgagggatgaagacgacagcaacagcaaggaagtcaccgatgccattatgcatgacgttgatacagacaaggtcagtatgtccatttatgtgcacgtacaccatttagtaacttttcgttctttggttcatacttcatagtgcaaatattatgcatcgatgctaatgtttacatcttaatgagttctcattttgaagttcccttctgaaccctcgtttgagtttagagctcaaatactccacctagttgtgacttattgtatctctaaagtccgtcatgttatataaattatactttgatggcatgcacactttctagcacatatgtgttgctaacgaaactctagtgctgtgacaaatgaaatacatgtatacattgatggcatctgttcttcccatcacatatgtactaccggtcattactctctagtgctgtgacattatgctctccctgttcgcttcatgatttacacggttggttaacctgctgctcgttacatgtctgctacagaattcaagttttgttagctgttttccatctcatctctaacaacatccccactcatttgtattagcttcttatctggagttttgtgagctcagtcttgtatcgtcttgtgtcaaaacgtgtgcaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 103 - DNA, 5526 bp, Solanum lycopersicum; identical to SEQ ID NO: 11
  source 1..5526: mol_type = genomic DNA

SEQ ID NO: 104 - DNA, 5517 bp, Solanum lycopersicum; identical to SEQ ID NO: 98
  source 1..5517: mol_type = genomic DNA

SEQ ID NO: 105 - DNA, 5501 bp, Solanum lycopersicum; identical to SEQ ID NO: 102
  source 1..5501: mol_type = genomic DNA

SEQ ID NO: 106 - DNA, 5517 bp, Solanum lycopersicum
  source 1..5517: mol_type = genomic DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcgtggaagtcgttcaggtatagtttttatcagcttcttagttctcagttacctactgcttttattaccttttgcttaggttatcttattatatccattattttcagcatagcttcttcattactgtatttccttttcatattgtttttatatatgtcttacttaggctgagggtccattggaaatagcctctctaccttcacaaggtaggagtaaggctgcgtacacactacccttcccacaccccacttgtgggactatactaagtatgttgttgttttagttcacaattgatgctttttggtttttatgtagtaacttctggtgtttcttttactgagttcttcaaagcttttgtatgggttttatatcatgacggatgtatcctgtattgttgtttcgggtttttattaaagaagtctggcaatttcaagttaaaaagaaaatcatatcttaaaagagaagcctaatcttgagcagatagcaatttcagggtgaaatgtgaatattcttggtgaagttgcatatgaacatatgaactggctaaccattattcaattcatttctagtgttgatctttgcaatcaatggtttcattctagtgtacgttcatgctgtagcatttaccttgatatcctcatggaatttcctttaccgccaaaattataatgagaagtggttatcttttttaataatggtaaagtatgagaagaggttttcttatgttctcagtctttgctagttctatttctttactcgttttacttaacttttttagtttcttgtgaactgtagtattgctctatatcttctcactcaaattcatgttctaaatcttaaccattctagtgatataaatgaagttaattggtgatataggatatccccctccaaaatctaacaatctttcaactcatcatagtcttgggcagatagctagttagaaccgtaacaaatatttaggaaagtagatcattcgaaatagttatccttgtgaaatattattctttatcttcattattgaccattggttttcaaactctagtactcttaagtggaagactcacttcttgtttctgtggagtattagctatagcggcctacaacagaatttgattgttgcactcaaagttaaacagagttctacagtttagggaaatgataatggaatttaaatttagtttgtatactgaagtaaatatggttgacactatcagaaacaagtgctttttacttcaaatattgatctttatcctcattgcgaaccattgatgtccaaactgttttagatggaagagcaatttgttgttcatttgagtggaatagtcaccaacaacagctaattaactacgtatagcttagagagatgcgaaccaatggtaaatttaagttgtctcctgaagtagttactgattgttcatgataaaggtcattaaagttgaattctctatttagtcagcgtccttttctgcatcgactatatagtggcctgaatgacattacaaaatatttatgagttggtaggaatctttttttcatatgtgaatcatgaatgtgttcacaaatgatcaatcagagagtgattattgtattatgtggtgttgataacttttgaacttttcttcatttggtttgtgtttaactgttatctgaaaggcttggttggtggtactaaagctattggattagcagaaaactttatacatgtttatggtcatgtttcgctctccagtctccgtttttatttgtcaaaggtcagttcatatatttccaatggttttgcagatgtgtcacaggcaaggggtaatgcatcgtgatctcaaacctgagaattttctttttggtaacaagaaagaaacagctccactgaaggctattgactttgggttgtctgttttctttaaacctggtaatggaattggaaaagaaacagtgagtttaagacttttatcatgctaaacaaaggtttaacattttgattattttaacaggggaacgctttaatgagatagtgggaagtccttattacatggctcctgaggtcctaaagcgcaattatggaccagaggtcgatgtctggagtgctggagttatactttacattcttctatgcggtgttccacctttctgggcaggtctgcttattttatttcttcgttttttataaccatggaacgaacctcaatgcagatattttcatgtgcaatttttggaacttgataattgattaactaagattgtgaatatgaagtagaacttgattcatgttagaatatgtcgttgaaatattagatgatgaggcaagtgaggtgaaagactgtccctgctttaatcaatgtagatcatgttcaattattttggtttctcttaatggttcttatgggttggactttgctaaataacttgcataaatatgttaacatattcttgatacttcccttaagttcctctgaatatttgcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttggtaatgtttgtggaatattgagatattgtactttattcattcgttgttttactctctctctctctctctatctatctatctctgataagtatttttctccggggctttgactcttgtggtgcagagcatccctggttacataatataaagaaagcaccaaacgtctcattgggtgagactgttaaagcaagactcaagcagttttcagtaatgaacaagctcaagaaaaaagctctgacggtaagcacacacttagtctgcaatcatatttataagctctggtttcttcactgtccactaaagatgaattcatatgcttttcctcatagatcgaaatgaaggagagaaggttgtgtcttaattatttactgcactgccttgtgctatttaataaaaacttaattctcatcgtaaaaaatgagaggcggttctcaaaattttgatcaagtttcgtttttttatcttgatatatgtactttcttgtattccaaacaggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggtaagcggtctcatgccctgcttcttggctcgatcagatttcttatagctctaccgggggtaacataatatttgaaagttagctgctaaacttttctgcctcaaagagaaaacaggtgcctgagatgatatggccatgtcttacgtagaactgtaaattacatcaatggacattattttaaaatgaccgaaaactacatggaatccatgtgaacttagtaaagaaaatattcaatggaagtaaaggataccaagtagttggattaaggctagattgttggtggtacactgacattagtgtctcaatgtttactaagagtctcagtttgattagagatttgcacgtagatgcagggataaccgtgagattaagaaaattttaagaaattcacggagatgaaaaagatctgtaatactcatatagcttagggccaagcccaactagtttgggatcaaatcgtagttgttgttgataagaatttagcattagacttttgtctgtcaccctactgtcaaaaaattgattgattacaaaatatattttgtcaatataagttgtaacatttttctaccatttcaatgtagaaaaaaggaaaatctgtagcaaaattagcttgacatttcgcttgtcttagcatccctggttcaatgcttattattatccagaaaggaaaaaaaaacatttctcttgatccttttgcatcttcgacctgaaggatgcttatcttttttattattttatttccttgaacttttgttttatgtttttcttattctgtatcggttttacaggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggtcagtatgtccatttatgtgcacgtacaccatttagtaacttttcgttctttggttcatacttcatagtgcaaatattatgcatcgatgctaatgtttacatcttaatgagttctcattttgaagttcccttctgaaccctcgtttgagtttagagctcaaatactccacctagttgtgacttattgtatctctaaagtccgtcatgttatataaacgtattatactttgatggcatgcacactttctagcacatatgtgttgctaacgaaactctagtgctgtgacaaatgaaatacatgtatacattgatggcatctgttcttcccatcacatatgtactaccggtcattactctctagtgctgtgacattatgctctccctgttcgcttcatgatttacacggttggttaacctgctgctcgttacatgtctgctacagaattcaagttttgttagctgttttccatctcatctctaacaacatccccactcatttgtattagcttcttatctggagttttgtgagctcagtcttgtatcgtcttgtgtcaaaacgtgtgcaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 107 - DNA, 5517 bp, Solanum lycopersicum; identical to SEQ ID NO: 106
  source 1..5517: mol_type = genomic DNA

SEQ ID NO: 108 - DNA, 5501 bp, Solanum lycopersicum; identical to SEQ ID NO: 102
  source 1..5501: mol_type = genomic DNA

SEQ ID NO: 109 - DNA, 1602 bp, Solanum lycopersicum; identical to SEQ ID NO: 9
  source 1..1602: mol_type = other DNA

SEQ ID NO: 110 - DNA, 1644 bp, Solanum lycopersicum
  source 1..1644: mol_type = other DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcgtggaagtcgttcagatgtgtcacaggcaaggggtaatgcatcgtgatctcaaacctgagaattttctttttggtaacaagaaagaaacagctccactgaaggctattgactttgggttgtctgttttctttaaacctggggaacgctttaatgagatagtgggaagtccttattacatggctcctgaggtcctaaagcgcaattatggaccagaggtcgatgtctggagtgctggagttatactttacattcttctatgcggtgttccacctttctgggcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttggtaatgtttgtggaatattgagatattgtactttattcattcagcatccctggttacataatataaagaaagcaccaaatgtctcattgggtgagactgttaaagcaagactcaagcagttttcagtaatgaacaagctcaagaaaaaagctctgacggttatagctgagtttttgtctgcggaggaagtcgctggaatgaaggaagcatttgaaatgatggataccggaaagaagggtaagataaacctgaatgaacttaaagatggcttgcagaagcttggccatcaaatccctgatgctgatcttcatattctcatggaagcggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 111 - DNA, 1602 bp, Solanum lycopersicum; identical to SEQ ID NO: 9
  source 1..1602: mol_type = other DNA

SEQ ID NO: 112 - DNA, 1602 bp, Solanum lycopersicum; identical to SEQ ID NO: 9
  source 1..1602: mol_type = other DNA

SEQ ID NO: 113 - DNA, 1602 bp, Solanum lycopersicum; identical to SEQ ID NO: 9
  source 1..1602: mol_type = other DNA

SEQ ID NO: 114 - DNA, 1602 bp, Solanum lycopersicum; identical to SEQ ID NO: 9
  source 1..1602: mol_type = other DNA

SEQ ID NO: 115 - DNA, 1602 bp, Solanum lycopersicum; identical to SEQ ID NO: 9
  source 1..1602: mol_type = other DNA

SEQ ID NO: 116 - DNA, 1602 bp, Solanum lycopersicum; identical to SEQ ID NO: 9
  source 1..1602: mol_type = other DNA

SEQ ID NO: 117 - DNA, 1602 bp, Solanum lycopersicum; identical to SEQ ID NO: 9
  source 1..1602: mol_type = other DNA

SEQ ID NO: 118 - DNA, 1635 bp, Solanum lycopersicum
  source 1..1635: mol_type = other DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcgtggaaatgtgtcacaggcaaggggtaatgcatcgtgatctcaaacctgagaattttctttttggtaacaagaaagaaacagctccactgaaggctattgactttgggttgtctgttttctttaaacctggggaacgctttaatgagatagtgggaagtccttattacatggctcctgaggtcctaaagcgcaattatggaccagaggtcgatgtctggagtgctggagttatactttacattcttctatgcggtgttccacctttctgggcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttggtaatgtttgtggaatattgagatattgtactttattcattcagcatccctggttacataatataaagaaagcaccaaacgtctcattgggtgagactgttaaagcaagactcaagcagttttcagtaatgaacaagctcaagaaaaaagctctgacggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 119 - DNA, 1602 bp, Solanum lycopersicum; identical to SEQ ID NO: 9
  source 1..1602: mol_type = other DNA

SEQ ID NO: 120 - DNA, 1602 bp, Solanum lycopersicum; identical to SEQ ID NO: 9
  source 1..1602: mol_type = other DNA

SEQ ID NO: 121 - DNA, 1626 bp, Solanum lycopersicum
  source 1..1626: mol_type = other DNA
atgggtaattgctgtgtaaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactatgtgtcacaggcaaggggtaatgcatcgtgatctcaaacctgagaattttctttttggtaacaagaaagaaacagctccactgaaggctattgactttgggttgtctgttttctttaaacctggggaacgctttaatgagatagtgggaagtccttattacatggctcctgaggtcctaaagcgcaattatggaccagaggtcgatgtctggagtgctggagttatactttacattcttctatgcggtgttccacctttctgggcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttggtaatgtttgtggaatattgagatattgtactttattcattcagcatccctggttacataatataaagaaagcaccaaacgtctcattgggtgagactgttaaagcaagactcaagcagttttcagtaatgaacaagctcaagaaaaaagctctgacggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 122 - DNA, 1602 bp, Solanum lycopersicum; identical to SEQ ID NO: 9
  source 1..1602: mol_type = other DNA

SEQ ID NO: 123 - DNA, 1602 bp, Solanum lycopersicum; identical to SEQ ID NO: 9
  source 1..1602: mol_type = other DNA

SEQ ID NO: 124 - DNA, 1587 bp, Solanum lycopersicum
  source 1..1587: mol_type = other DNA
atgggtaattgctgtgtaaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcatgtgtcacaggcaaggggtaatgcatcgtgatctcaaacctgagaattttctttttggtaacaagaaagaaacagctccactgaaggctattgactttgggttgtctgttttctttaaacctggggaacgctttaatgagatagtgggaagtccttattacatggctcctgaggtcctaaagcgcaattatggaccagaggtcgatgtctggagtgctggagttatactttacattcttctatgcggtgttccacctttctgggcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttgagcatccctggttacataatataaagaaagcaccaaacgtctcattgggtgagactgttaaagcaagactcaagcagttttcagtaatgaacaagctcaagaaaaaagctctgacggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 125 - DNA, 1602 bp, Solanum lycopersicum; identical to SEQ ID NO: 9
  source 1..1602: mol_type = other DNA

SEQ ID NO: 126 - DNA, 1788 bp, Solanum lycopersicum
  source 1..1788: mol_type = other DNA
atgaatttcttttgtggacaaagtgctcttattggattgttcaatgaagtttcgacactgaggttaggtataaagtctaattatggtgtttcaatattgggaggaatgcttcgcttttactatactcattatcaatggcagatggacggatctttcttttttctcttcttttttaccccttttttggtctataattggaaaaaaatcaacaattttcaggagttggttgatcttataaagtatcatggcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcgtggaagtcgttcagatgtgtcacaggcaaggggtaatgcatcgtgatctcaaacctgagaattttctttttggtaacaagaaagaaacagctccactgaaggctattgactttgggttgtctgttttctttaaacctggggaacgctttaatgagatagtgggaagtccttattacatggctcctgaggtcctaaagcgcaattatggaccagaggtcgatgtctggagtgctggagttatactttacattcttctatgcggtgttccacctttctgggcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttgagcatccctggttacataatataaagaaagcaccaaatgtctcattgggtgagactgttaaagcaagactcaagcagttttcagtaatgaacaagctcaagaaaaaagctctgacggttatagctgagtttttgtctgcggaggaagtcgctggaatgaaggaagcatttgaaatgatggataccggaaagaagggtaagataaacctgaatgaacttaaagatggcttgcagaagcttggccatcaaatccctgatgctgatcttcatattctcatggaagcggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 127 - DNA, 1602 bp, Solanum lycopersicum; identical to SEQ ID NO: 9
  source 1..1602: mol_type = other DNA

SEQ ID NO: 128 - DNA, 1584 bp, Solanum lycopersicum
  source 1..1584: mol_type = other DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcatgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactatgtgtcacaggcaaggggtaatgcatcgtgatctcaaacctgagaattttctttttggtaacaagaaagaaacagctccactgaaggctattgactttgggttgtctgttttctttaaacctggggaacgctttaatgagatagtgggaagtccttattacatggctcctgaggtcctaaagcgcaattatggaccagaggtcgatgtctggagtgctggagttatactttacattcttctatgcggtgttccacctttctgggcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttgagcatccctggttacataatataaagaaagcaccaaacgtctcattgggtgagactgttaaagcaagactcaagcagttttcagtaatgaacaagctcaagaaaaaagctctgacggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 129 - DNA, 1602 bp, Solanum lycopersicum; identical to SEQ ID NO: 9
  source 1..1602: mol_type = other DNA

SEQ ID NO: 130 - DNA, 1602 bp, Solanum lycopersicum; identical to SEQ ID NO: 9
  source 1..1602: mol_type = other DNA

SEQ ID NO: 131 - DNA, 1602 bp, Solanum lycopersicum; identical to SEQ ID NO: 9
  source 1..1602: mol_type = other DNA

SEQ ID NO: 132 - DNA, 1602 bp, Solanum lycopersicum; identical to SEQ ID NO: 9
  source 1..1602: mol_type = other DNA

SEQ ID NO: 133 - DNA, 1587 bp, Solanum lycopersicum
  source 1..1587: mol_type = other DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcatgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcatgtgtcacaggcaaggggtaatgcatcgtgatctcaaacctgagaattttctttttggtaacaagaaagaaacagctccactgaaggctattgactttgggttgtctgttttctttaaacctggggaacgctttaatgagatagtgggaagtccttattacatggctcctgaggtcctaaagcgcaattatggaccagaggtcgatgtctggagtgctggagttatactttacattcttctatgcggtgttccacctttctgggcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttgagcatccctggttacataatataaagaaagcaccaaacgtctcattgggtgagactgttaaagcaagactcaagcagttttcagtaatgaacaagctcaagaaaaaagctctgacggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 134 - DNA, 1602 bp, Solanum lycopersicum; identical to SEQ ID NO: 9
  source 1..1602: mol_type = other DNA

SEQ ID NO: 135 - DNA, 1602 bp, Solanum lycopersicum; identical to SEQ ID NO: 9
  source 1..1602: mol_type = other DNA

SEQ ID NO: 136 - DNA, 1602 bp, Solanum lycopersicum; identical to SEQ ID NO: 9
  source 1..1602: mol_type = other DNA

SEQ ID NO: 137 - DNA, 1614 bp, Solanum lycopersicum
  source 1..1614: mol_type = other DNA
atggcggatttttcttttttctcttcttttttaccccttttttggtctataattggaaaaaaatcaacaattttcagtatcatgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcatgtgtcacaggcaaggggtaatgcatcgtgatctcaaacctgagaattttctttttggtaacaagaaagaaacagctccactgaaggctattgactttgggttgtctgttttctttaaacctggggaacgctttaatgagatagtgggaagtccttattacatggctcctgaggtcctaaagcgcaattatggaccagaggtcgatgtctggagtgctggagttatactttacattcttctatgcggtgttccacctttctgggcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttgagcatccctggttacataatataaagaaagcaccaaacgtctcattgggtgagactgttaaagcaagactcaagcagttttcagtaatgaacaagctcaagaaaaaagctctgacggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcagattctcatggaagcggctgacgttgatggagatggaagtttaaattatccagagtttgttgctgtatctattcatcttagaaagatggccaatgatgaacacctgcacaaagcattttcattttttgacaaaaaccaaagtggtttcatagaaatcgaagagcttcgtagtgctttgagggatgaagacgacagcaacagcaaggaagtcaccgatgccattatgcatgacgttgatacagacaaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 138 - DNA, 1602 bp, Solanum lycopersicum; identical to SEQ ID NO: 9
  source 1..1602: mol_type = other DNA

SEQ ID NO: 139 - DNA, 1602 bp, Solanum lycopersicum; identical to SEQ ID NO: 9
  source 1..1602: mol_type = other DNA

SEQ ID NO: 140 - DNA, 1602 bp, Solanum lycopersicum; identical to SEQ ID NO: 9
  source 1..1602: mol_type = other DNA

SEQ ID NO: 141 - DNA, 1584 bp, Solanum lycopersicum
  source 1..1584: mol_type = other DNA
atgggtaattgctgtgtaaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactatgtgtcacaggcaaggggtaatgcatcgtgatctcaaacctgagaattttctttttggtaacaagaaagaaacagctccactgaaggctattgactttgggttgtctgttttctttaaacctggggaacgctttaatgagatagtgggaagtccttattacatggctcctgaggtcctaaagcgcaattatggaccagaggtcgatgtctggagtgctggagttatactttacattcttctatgcggtgttccacctttctgggcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttgagcatccctggttacataatataaagaaagcaccaaacgtctcattgggtgagactgttaaagcaagactcaagcagttttcagtaatgaacaagctcaagaaaaaagctctgacggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 142 - DNA, 1602 bp, Solanum lycopersicum; identical to SEQ ID NO: 9
  source 1..1602: mol_type = other DNA

SEQ ID NO: 143 - DNA, 1587 bp, Solanum lycopersicum; identical to SEQ ID NO: 124
  source 1..1587: mol_type = other DNA

SEQ ID NO: 144 - DNA, 1479 bp, Solanum lycopersicum
  source 1..1479: mol_type = other DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcatgtgtcacaggcaaggggtaatgcatcgtgatctcaaacctgagaattttctttttggtaacaagaaagaaacagctccactgaaggctattgactttgggttgtctgttttctttaaacctggggaacgctttaatgagatagtgggaagtccttattacatggctcctgaggtcctaaagcgcaattatggaccagaggtcgatgtctggagtgctggagttatactttacattcttctatgcggtgttccacctttctgggcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttgagcatccctggttacataatataaagaaagcaccaaacgtctcattgggtgagactgttaaagcaagactcaagcagttttcagtaatgaacaagctcaagaaaaaagctctgacggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcagattctcatggaagcggctgacgttgatggagatggaagtttaaattatccagagtttgttgctgtatctattcatcttagaaagatggccaatgatgaacacctgcacaaagcattttcattttttgacaaaaaccaaagtggtttcatagaaatcgaagagcttcgtagtgctttgagggatgaagacgacagcaacagcaaggaagtcaccgatgccattatgcatgacgttgatacagacaaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 145 - DNA, 1602 bp, Solanum lycopersicum; identical to SEQ ID NO: 9
  source 1..1602: mol_type = other DNA

SEQ ID NO: 146 - DNA, 1647 bp, Solanum lycopersicum
  source 1..1647: mol_type = other DNA
atggcggatttttcttttttctcttcttttttaccccttttttggtctataattggaaaaaaatcaacaattttcagtatcatgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcatgtgtcacaggcaaggggtaatgcatcgtgatctcaaacctgagaattttctttttggtaacaagaaagaaacagctccactgaaggctattgactttgggttgtctgttttctttaaacctggggaacgctttaatgagatagtgggaagtccttattacatggctcctgaggtcctaaagcgcaattatggaccagaggtcgatgtctggagtgctggagttatactttacattcttctatgcggtgttccacctttctgggcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttggtaataaagcaccaaacgtctcattgggtgagactgttaaagcaagactcaagcagttttcagtaatgaacaagctcaagaaaaaagctctgacggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 147 - DNA, 1602 bp, Solanum lycopersicum; identical to SEQ ID NO: 9
  source 1..1602: mol_type = other DNA

SEQ ID NO: 148 - DNA, 1587 bp, Solanum lycopersicum
  source 1..1587: mol_type = other DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactgtcatgtgtcacaggcaaggggtaatgcatcgtgatctcaaacctgagaattttctttttggtaacaagaaagaaacagctccactgaaggctattgactttgggttgtctgttttctttaaacctggggaacgctttaatgagatagtgggaagtccttattacatggctcctgaggtcctaaagcgcaattatggaccagaggtcgatgtctggagtgctggagttatactttacattcttctatgcggtgttccacctttctgggcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttgagcatccctggttacataatataaagaaagcaccaaacgtctcattgggtgagactgttaaagcaagactcaagcagttttcagtaatgaacaagctcaagaaaaaagctctgacggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 149 - DNA, 1602 bp, Solanum lycopersicum; identical to SEQ ID NO: 9
  source 1..1602: mol_type = other DNA

SEQ ID NO: 150 - DNA, 1602 bp, Solanum lycopersicum; identical to SEQ ID NO: 9
  source 1..1602: mol_type = other DNA

SEQ ID NO: 151 - DNA, 1602 bp, Solanum lycopersicum
  source 1..1602: mol_type = other DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcgtgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagggcagctgctggaattttgaagactgtcgtggaagttgttcagatgtgtcacaggcaaggggtaatgcatcgtgatctcaaacctgagaattttctttttggtaacaagaaagaaacagctccactgaaggctattgactttgggttgtctgttttctttaaacctggggaacgctttaatgagatagtgggaagtccttattacatggctcctgaggtcctaaagcgcaattatggaccagaggtcgatgtctggagtgctggagttatactttacattcttctatgcggtgttccacctttctgggcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttgagcatccctggttacataatataaagaaagcaccaaacgtctcattgggtgagactgttaaagcaagactcaagcagttttcagtaatgaacaagctcaagaaaaaagctctgacggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcagattctcatggaagcggctgacgttgatggagatggaagtttaaattatccagagtttgttgctgtatctattcatcttagaaagatggccaatgatgaacacctgcacaaagcattttcattttttgacaaaaaccaaagtggtttcatagaaatcgaagagcttcgtagtgctttgagggatgaagacgacagcaacagcaaggaagtcaccgatgccattatgcatgacgttgatacagacaaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 152 - DNA, 1626 bp, Solanum lycopersicum
  source 1..1626: mol_type = other DNA
atgggtaattgctgtgtgaaaccgggtaaatctgccgaaaaaaagaataaaaagaacaacagcaaacctaaccctttttcaattgattatggggggactaaacatgcctctgggagtggaaacaagctagttgtattaaaagaaccaacagggcaaaatatacatgataagtatgatttggggcatgagctcggaagaggagaatttggggttacttatctatgtactgacttggaaggaggagaaaaatatgcttgcaaatcgatatctaaaaagaagctaaggactgcagtggatattgatgatgttaggagagaggttgagatcatgaaacatttgcctgtgcatccgaatattgtgaccttgaaggatacttatgaggatgataatgcagtgcacattgtgatggaattgtgtgagggtggggagttgtttgatcgaatcgttgctagaggccactatacagagagagcagctgctggaattttgaagactatgtgtcacaggcaaggggtaatgcatcgtgatctcaaacctgagaattttctttttggtaacaagaaagaaacagctccactgaaggctattgactttgggttgtctgttttctttaaacctggggaacgctttaatgagatagtgggaagtccttattacatggctcctgaggtcctaaagcgcaattatggaccagaggtcgatgtctggagtgctggagttatactttacattcttctatgcggtgttccacctttctgggcagagactgaacaaggagtagcccaagcgattattcgttctgtgattgatttcaagagggatccatggcctaaagtttctgataatgcaaaggatcttgtaaagaaaatgcttgatccagatccaactcgacggctcacagctcatcaagttcttggtaatgtttgtggaatattgagatattgtactttattcattcagcatccctggttacataatataaagaaagcaccaaacgtctcattgggtgagactgttaaagcaagactcaagcagttttcagtaatgaacaagctcaagaaaaaagctctgacggttatagctgagtttttgtctgcggaggaagtcgctggaatgaaggaagcatttgaaatgatggataccggaaagaagggcaagataaacctgaatgaacttaaagatggcttgcagaagcttggccatcaaatccctgatgctgatcttcatattctcatggaagcggctgacgttgatggagatggaagtttaaattatccagagtttgttgctgtatctattcatcttagaaagatggccaatgatgaacacctgcacaaagcattttcatttttcgacaaaaatcagagtggtttcatagaaatcgaagagcttcgtagtgctttgagggatgaagacgacagcaacagcgaggaagtcaccaatgccattatgcatgacgttgatacagacaaggatggtcggattagttatgaggaatttgctgcgatgatgaaggctggtacggactggagaaaagcatcgagacagtattctcgtgaacgttttaacagtctaagcttaaagttgatgagggaaggctcattacaagttgaaaacaaagtctag

SEQ ID NO: 153 - protein, 533 aa, Solanum lycopersicum; identical to SEQ ID NO: 10

SEQ ID NO: 154 - protein, 547 aa, Solanum lycopersicum
MGNCCVKPGKSAEKKNKKNNSKPNPFSIDYGGTKHASGSGNKLVVLKEPTGQNIHDKYDLGRELGRGEFGVTYLCTDLEGGEKYACKSISKKKLRTAVDIDDVRREVEIMKHLPVHPNIVTLKDTYEDDNAVHIVMELCEGGELFDRIVARGHYTERAAAGILKTVVEVVQMCHRQGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVIDFKRDPWPKVSDNAKDLVKKMLDPDPTRRLTAHQVLGNVCGILRYCTLFIQHPWLHNIKKAPNVSLGETVKARLKQFSVMNKLKKKALTVIAEFLSAEEVAGMKEAFEMMDTGKKGKINLNELKDGLQKLGHQIPDADLHILMEAADVDGDGSLNYPEFVAVSIHLRKMANDEHLHKAFSFFDKNQSGFIEIEELRSALRDEDDSNSEEVTNAIMHDVDTDKDGRISYEEFAAMMKAGTDWRKASRQYSRERFNSLSLKLMREGSLQVENKV

SEQ ID NO: 155 - protein, 533 aa, Solanum lycopersicum; identical to SEQ ID NO: 10

SEQ ID NO: 156 - protein, 533 aa, Solanum lycopersicum; identical to SEQ ID NO: 10

SEQ ID NO: 157 - protein, 533 aa, Solanum lycopersicum; identical to SEQ ID NO: 10

SEQ ID NO: 158 - protein, 533 aa, Solanum lycopersicum; identical to SEQ ID NO: 10

SEQ ID NO: 159 - protein, 533 aa, Solanum lycopersicum; identical to SEQ ID NO: 10

SEQ ID NO: 160 - protein, 533 aa, Solanum lycopersicum; identical to SEQ ID NO: 10

SEQ ID NO: 161 - protein, 533 aa, Solanum lycopersicum; identical to SEQ ID NO: 10

SEQ ID NO: 162 - protein, 544 aa, Solanum lycopersicum
MGNCCVKPGKSAEKKNKKNNSKPNPFSIDYGGTKHASGSGNKLVVLKEPTGQNIHDKYDLGRELGRGEFGVTYLCTDLEGGEKYACKSISKKKLRTAVDIDDVRREVEIMKHLPVHPNIVTLKDTYEDDNAVHIVMELCEGGELFDRIVARGHYTERAAAGILKTVVEMCHRQGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVIDFKRDPWPKVSDNAKDLVKKMLDPDPTRRLTAHQVLGNVCGILRYCTLFIQHPWLHNIKKAPNVSLGETVKARLKQFSVMNKLKKKALTVIAEFLSAEEVAGMKEAFEMMDTGKKGKINLNELKDGLQKLGHQIPDADLHILMEAADVDGDGSLNYPEFVAVSIHLRKMANDEHLHKAFSFFDKNQSGFIEIEELRSALRDEDDSNSEEVTNAIMHDVDTDKDGRISYEEFAAMMKAGTDWRKASRQYSRERFNSLSLKLMREGSLQVENKV

SEQ ID NO: 163 - protein, 533 aa, Solanum lycopersicum; identical to SEQ ID NO: 10

SEQ ID NO: 164 - protein, 533 aa, Solanum lycopersicum; identical to SEQ ID NO: 10

SEQ ID NO: 165 - protein, 541 aa, Solanum lycopersicum
MGNCCVKPGKSAEKKNKKNNSKPNPFSIDYGGTKHASGSGNKLVVLKEPTGQNIHDKYDLGRELGRGEFGVTYLCTDLEGGEKYACKSISKKKLRTAVDIDDVRREVEIMKHLPVHPNIVTLKDTYEDDNAVHIVMELCEGGELFDRIVARGHYTERAAAGILKTMCHRQGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVIDFKRDPWPKVSDNAKDLVKKMLDPDPTRRLTAHQVLGNVCGILRYCTLFIQHPWLHNIKKAPNVSLGETVKARLKQFSVMNKLKKKALTVIAEFLSAEEVAGMKEAFEMMDTGKKGKINLNELKDGLQKLGHQIPDADLHILMEAADVDGDGSLNYPEFVAVSIHLRKMANDEHLHKAFSFFDKNQSGFIEIEELRSALRDEDDSNSEEVTNAIMHDVDTDKDGRISYEEFAAMMKAGTDWRKASRQYSRERFNSLSLKLMREGSLQVENKV

SEQ ID NO: 166 - protein, 533 aa, Solanum lycopersicum; identical to SEQ ID NO: 10

SEQ ID NO: 167 - protein, 533 aa, Solanum lycopersicum; identical to SEQ ID NO: 10

SEQ ID NO: 168 - protein, 528 aa, Solanum lycopersicum
MGNCCVKPGKSAEKKNKKNNSKPNPFSIDYGGTKHASGSGNKLVVLKEPTGQNIHDKYDLGRELGRGEFGVTYLCTDLEGGEKYACKSISKKKLRTAVDIDDVRREVEIMKHLPVHPNIVTLKDTYEDDNAVHIVMELCEGGELFDRIVARGHYTERAAAGILKTVMCHRQGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVIDFKRDPWPKVSDNAKDLVKKMLDPDPTRRLTAHQVLEHPWLHNIKKAPNVSLGETVKARLKQFSVMNKLKKKALTVIAEFLSAEEVAGMKEAFEMMDTGKKGKINLNELKDGLQKLGHQIPDADLHILMEAADVDGDGSLNYPEFVAVSIHLRKMANDEHLHKAFSFFDKNQSGFIEIEELRSALRDEDDSNSEEVTNAIMHDVDTDKDGRISYEEFAAMMKAGTDWRKASRQYSRERFNSLSLKLMREGSLQVENKV

SEQ ID NO: 169 - protein, 533 aa, Solanum lycopersicum; identical to SEQ ID NO: 10

SEQ ID NO: 170 - protein, 595 aa, Solanum lycopersicum
MNFFCGQSALIGLFNEVSTLRLGIKSNYGVSILGGMLRFYYTHYQWQMDGSFFFLFFFTPFLVYNWKKINNFQELVDLIKYHGKPNPFSIDYGGTKHASGSGNKLVVLKEPTGQNIHDKYDLGRELGRGEFGVTYLCTDLEGGEKYACKSISKKKLRTAVDIDDVRREVEIMKHLPVHPNIVTLKDTYEDDNAVHIVMELCEGGELFDRIVARGHYTERAAAGILKTVVEVVQMCHRQGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVIDFKRDPWPKVSDNAKDLVKKMLDPDPTRRLTAHQVLEHPWLHNIKKAPNVSLGETVKARLKQFSVMNKLKKKALTVIAEFLSAEEVAGMKEAFEMMDTGKKGKINLNELKDGLQKLGHQIPDADLHILMEAADVDGDGSLNYPEFVAVSIHLRKMANDEHLHKAFSFFDKNQSGFIEIEELRSALRDEDDSNSEEVTNAIMHDVDTDKDGRISYEEFAAMMKAGTDWRKASRQYSRERFNSLSLKLMREGSLQVENKV

SEQ ID NO: 171 - protein, 533 aa, Solanum lycopersicum; identical to SEQ ID NO: 10

SEQ ID NO: 172 - protein, 527 aa, Solanum lycopersicum
MGNCCVKPGKSAEKKNKKNNSKPNPFSIDYGGTKHASGSGNKLVVLKEPTGQNIHDKYDLGHELGRGEFGVTYLCTDLEGGEKYACKSISKKKLRTAVDIDDVRREVEIMKHLPVHPNIVTLKDTYEDDNAVHIVMELCEGGELFDRIVARGHYTERAAAGILKTMCHRQGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVIDFKRDPWPKVSDNAKDLVKKMLDPDPTRRLTAHQVLEHPWLHNIKKAPNVSLGETVKARLKQFSVMNKLKKKALTVIAEFLSAEEVAGMKEAFEMMDTGKKGKINLNELKDGLQKLGHQIPDADLHILMEAADVDGDGSLNYPEFVAVSIHLRKMANDEHLHKAFSFFDKNQSGFIEIEELRSALRDEDDSNSEEVTNAIMHDVDTDKDGRISYEEFAAMMKAGTDWRKASRQYSRERFNSLSLKLMREGSLQVENKV

SEQ ID NO: 173 - protein, 533 aa, Solanum lycopersicum; identical to SEQ ID NO: 10

SEQ ID NO: 174 - protein, 533 aa, Solanum lycopersicum; identical to SEQ ID NO: 10

SEQ ID NO: 175 - protein, 533 aa, Solanum lycopersicum; identical to SEQ ID NO: 10

SEQ ID NO: 176 - protein, 533 aa, Solanum lycopersicum; identical to SEQ ID NO: 10

SEQ ID NO: 177 - protein, 528 aa, Solanum lycopersicum
MGNCCVKPGKSAEKKNKKNNSKPNPFSIDYGGTKHASGSGNKLVVLKEPTGQNIHDKYDLGHELGRGEFGVTYLCTDLEGGEKYACKSISKKKLRTAVDIDDVRREVEIMKHLPVHPNIVTLKDTYEDDNAVHIVMELCEGGELFDRIVARGHYTERAAAGILKTVMCHRQGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVIDFKRDPWPKVSDNAKDLVKKMLDPDPTRRLTAHQVLEHPWLHNIKKAPNVSLGETVKARLKQFSVMNKLKKKALTVIAEFLSAEEVAGMKEAFEMMDTGKKGKINLNELKDGLQKLGHQIPDADLHILMEAADVDGDGSLNYPEFVAVSIHLRKMANDEHLHKAFSFFDKNQSGFIEIEELRSALRDEDDSNSEEVTNAIMHDVDTDKDGRISYEEFAAMMKAGTDWRKASRQYSRERFNSLSLKLMREGSLQVENKV

SEQ ID NO: 178 - protein, 533 aa, Solanum lycopersicum; identical to SEQ ID NO: 10

SEQ ID NO: 179 - protein, 533 aa, Solanum lycopersicum; identical to SEQ ID NO: 10

SEQ ID NO: 180 - protein, 533 aa, Solanum lycopersicum; identical to SEQ ID NO: 10

SEQ ID NO: 181 - protein, 537 aa, Solanum lycopersicum
MADFSFFSSFLPLFWSIIGKKSTIFSIMGNCCVKPGKSAEKKNKKNNSKPNPFSIDYGGTKHASGSGNKLVVLKEPTGQNIHDKYDLGRELGRGEFGVTYLCTDLEGGEKYACKSISKKKLRTAVDIDDVRREVEIMKHLPVHPNIVTLKDTYEDDNAVHIVMELCEGGELFDRIMCHRQGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVIDFKRDPWPKVSDNAKDLVKKMLDPDPTRRLTAHQVLEHPWLHNIKKAPNVSLGETVKARLKQFSVMNKLKKKALTVIAEFLSAEEVAGMKEAFEMMDTGKKGKINLNELKDGLQKLGHQIPDADLQILMEAADVDGDGSLNYPEFVAVSIHLRKMANDEHLHKAFSFFDKNQSGFIEIEELRSALRDEDDSNSKEVTDAIMHDVDTDKDGRISYEEFAAMMKAGTDWRKASRQYSRERFNSLSLKLMREGSLQVENKV

SEQ ID NO: 182 - protein, 533 aa, Solanum lycopersicum; identical to SEQ ID NO: 10

SEQ ID NO: 183 - protein, 533 aa, Solanum lycopersicum; identical to SEQ ID NO: 10

SEQ ID NO: 184 - protein, 533 aa, Solanum lycopersicum; identical to SEQ ID NO: 10

SEQ ID NO: 185 - protein, 527 aa, Solanum lycopersicum
MGNCCVKPGKSAEKKNKKNNSKPNPFSIDYGGTKHASGSGNKLVVLKEPTGQNIHDKYDLGRELGRGEFGVTYLCTDLEGGEKYACKSISKKKLRTAVDIDDVRREVEIMKHLPVHPNIVTLKDTYEDDNAVHIVMELCEGGELFDRIVARGHYTERAAAGILKTMCHRQGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVIDFKRDPWPKVSDNAKDLVKKMLDPDPTRRLTAHQVLEHPWLHNIKKAPNVSLGETVKARLKQFSVMNKLKKKALTVIAEFLSAEEVAGMKEAFEMMDTGKKGKINLNELKDGLQKLGHQIPDADLHILMEAADVDGDGSLNYPEFVAVSIHLRKMANDEHLHKAFSFFDKNQSGFIEIEELRSALRDEDDSNSEEVTNAIMHDVDTDKDGRISYEEFAAMMKAGTDWRKASRQYSRERFNSLSLKLMREGSLQVENKV

SEQ ID NO: 186 - protein, 533 aa, Solanum lycopersicum; identical to SEQ ID NO: 10

SEQ ID NO: 187 - protein, 528 aa, Solanum lycopersicum; identical to SEQ ID NO: 168

SEQ ID NO: 188 - protein, 492 aa, Solanum lycopersicum
MGNCCVKPGKSAEKKNKKNNSKPNPFSIDYGGTKHASGSGNKLVVLKEPTGQNIHDKYDLGRGEKYACKSISKKKLRTAVDIDDVRREVEIMKHLPVHPNIVTLKDTYEDDNAVHIVMELCEGGELFDRIMCHRQGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVIDFKRDPWPKVSDNAKDLVKKMLDPDPTRRLTAHQVLEHPWLHNIKKAPNVSLGETVKARLKQFSVMNKLKKKALTVIAEFLSAEEVAGMKEAFEMMDTGKKGKINLNELKDGLQKLGHQIPDADLQILMEAADVDGDGSLNYPEFVAVSIHLRKMANDEHLHKAFSFFDKNQSGFIEIEELRSALRDEDDSNSKEVTDAIMHDVDTDKDGRISYEEFAAMMKAGTDWRKASRQYSRERFNSLSLKLMREGSLQVENKV

SEQ ID NO: 189 - protein, 533 aa, Solanum lycopersicum; identical to SEQ ID NO: 10

SEQ ID NO: 190 - protein, 548 aa, Solanum lycopersicum
MADFSFFSSFLPLFWSIIGKKSTIFSIMGNCCVKPGKSAEKKNKKNNSKPNPFSIDYGGTKHASGSGNKLVVLKEPTGQNIHDKYDLGRELGRGEFGVTYLCTDLEGGEKYACKSISKKKLRTAVDIDDVRREVEIMKHLPVHPNIVTLKDTYEDDNAVHIVMELCEGGELFDRIVARGHYTERAAAGILKTVMCHRQGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVIDFKRDPWPKVSDNAKDLVKKMLDPDPTRRLTAHQVLGNKAPNVSLGETVKARLKQFSVMNKLKKKALTVIAEFLSAEEVAGMKEAFEMMDTGKKGKINLNELKDGLQKLGHQIPDADLHILMEAADVDGDGSLNYPEFVAVSIHLRKMANDEHLHKAFSFFDKNQSGFIEIEELRSALRDEDDSNSEEVTNAIMHDVDTDKDGRISYEEFAAMMKAGTDWRKASRQYSRERFNSLSLKLMREGSLQVENKV

SEQ ID NO: 191 - protein, 533 aa, Solanum lycopersicum; identical to SEQ ID NO: 10

SEQ ID NO: 192 - protein, 528 aa, Solanum lycopersicum; identical to SEQ ID NO: 168

SEQ ID NO: 193 - protein, 533 aa, Solanum lycopersicum; identical to SEQ ID NO: 10

SEQ ID NO: 194 - protein, 533 aa, Solanum lycopersicum; identical to SEQ ID NO: 10

SEQ ID NO: 195 - protein, 533 aa, Solanum lycopersicum
MGNCCVKPGKSAEKKNKKNNSKPNPFSIDYGGTKHASGSGNKLVVLKEPTGQNIHDKYDLGRELGRGEFGVTYLCTDLEGGEKYACKSISKKKLRTAVDIDDVRREVEIMKHLPVHPNIVTLKDTYEDDNAVHIVMELCEGGELFDRIVARGHYTERAAAGILKTVVEVVQMCHRQGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVIDFKRDPWPKVSDNAKDLVKKMLDPDPTRRLTAHQVLEHPWLHNIKKAPNVSLGETVKARLKQFSVMNKLKKKALTVIAEFLSAEEVAGMKEAFEMMDTGKKGKINLNELKDGLQKLGHQIPDADLQILMEAADVDGDGSLNYPEFVAVSIHLRKMANDEHLHKAFSFFDKNQSGFIEIEELRSALRDEDDSNSKEVTDAIMHDVDTDKDGRISYEEFAAMMKAGTDWRKASRQYSRERFNSLSLKLMREGSLQVENKV

SEQ ID NO: 196 - protein, 541 aa, Solanum lycopersicum
MGNCCVKPGKSAEKKNKKNNSKPNPFSIDYGGTKHASGSGNKLVVLKEPTGQNIHDKYDLGHELGRGEFGVTYLCTDLEGGEKYACKSISKKKLRTAVDIDDVRREVEIMKHLPVHPNIVTLKDTYEDDNAVHIVMELCEGGELFDRIVARGHYTERAAAGILKTMCHRQGVMHRDLKPENFLFGNKKETAPLKAIDFGLSVFFKPGERFNEIVGSPYYMAPEVLKRNYGPEVDVWSAGVILYILLCGVPPFWAETEQGVAQAIIRSVIDFKRDPWPKVSDNAKDLVKKMLDPDPTRRLTAHQVLGNVCGILRYCTLFIQHPWLHNIKKAPNVSLGETVKARLKQFSVMNKLKKKALTVIAEFLSAEEVAGMKEAFEMMDTGKKGKINLNELKDGLQKLGHQIPDADLHILMEAADVDGDGSLNYPEFVAVSIHLRKMANDEHLHKAFSFFDKNQSGFIEIEELRSALRDEDDSNSEEVTNAIMHDVDTDKDGRISYEEFAAMMKAGTDWRKASRQYSRERFNSLSLKLMREGSLQVENKV

(19) United States
(12) Patent Application Publication
(10) Pub. No.: US 2026/0103719 A1
(43) Pub. Date: Apr. 16, 2026

(54) POLYNUCLEOTIDE, PROTEIN, BIOLOGICAL MATERIAL, AND USE THEREOF IN IMPROVING QUALITY OF PLANT FRUIT

(71) Applicants: AGRICULTURAL GENOMICS INSTITUTE, CHINESE ACADEMY OF AGRICULTURAL SCIENCES, Shenzhen (CN); INSTITUTE OF VEGETABLES AND FLOWERS, CHINESE ACADEMY OF AGRICULTURAL SCIENCES, Beijing (CN); AGRICULTURAL GENOMICS INSTITUTE AT SHENZHEN, CHINESE ACADEMY OF AGRICULTURAL SCIENCES, Shenzhen (CN)

(72) Inventors: Sanwen HUANG, Shenzhen (CN); Jinzhe ZHANG, Shenzhen (CN); Hongjun LYU, Shenzhen (CN); Jie CHEN, Shenzhen (CN); Guangtao ZHU, Shenzhen (CN)

(73) Assignees: AGRICULTURAL GENOMICS INSTITUTE, CHINESE ACADEMY OF AGRICULTURAL SCIENCES, Shenzhen (CN); INSTITUTE OF VEGETABLES AND FLOWERS, CHINESE ACADEMY OF AGRICULTURAL SCIENCES, Beijing (CN); AGRICULTURAL GENOMICS INSTITUTE AT SHENZHEN, CHINESE ACADEMY OF AGRICULTURAL SCIENCES, Shenzhen (CN)

(21) Appl. No.: 19/234,630

(22) Filed: Jun. 11, 2025

Related U.S. Application Data
(63) Continuation of application No. PCT/CN2023/138699, filed on Dec. 14, 2023.

(30) Foreign Application Priority Data
Dec. 15, 2022 (CN) 202211616622.8

(51) Int. Cl. C12N 15/82; C07K 14/415; C12N 9/10; C12N 9/12

(52) U.S. Cl. CPC C12N 15/8245; C07K 14/415; C12N 9/1062; C12N 9/12; C12N 15/8261; C12Y 204/01013; C12Y 207/11001; C12Y 207/11022

(57) ABSTRACT

Disclosed are a polynucleotide, a protein, a biological material, and use thereof in the improvement of the quality of a plant fruit. The polynucleotide includes any one of the sequences set forth in SEQ ID NOs: 3, 11, 23-36, and 95-108; a complementary sequence, a degenerate sequence, or a homologous sequence thereof; a sequence hybridized with the sequence or a complementary sequence thereof under rigorous conditions; and a cDNA sequence of the sequence. It is found that the gene is a key regulatory gene affecting the sugar content of the fruit, and in the use, the content of SSC, glucose, fructose, citric acid, and malic acid of the plant fruit can be regulated by regulating the protein function, the expression level, and the like of CDPK gene without significantly affecting the weight of the fruit, such that the quality and flavor of a tomato fruit are improved.

### CROSS-REFERENCE TO RELATED APPLICATION

The present application is a continuation of International Application No. PCT/CN2023/138699, filed on Dec. 14, 2023, which claims priority to Chinese Patent Application No. 202211616622.8, filed on Dec. 15, 2022. The disclosures of the aforementioned applications are hereby incorporated by reference in their entireties.

### REFERENCE TO AN ELECTRONIC SEQUENCE LISTING

The contents of the electronic sequence listing (SequenceListing.xml; Size: 496,870 bytes; and Date of Creation: Apr. 18, 2025) is herein incorporated by reference in its entirety.

### TECHNICAL FIELD

The present disclosure relates to the field of biotechnologies, and particularly to a polynucleotide, protein, biological material and use thereof in the improvement of the quality of plant fruits.

### BACKGROUND

Tomato, originated in the Andes region of South America, is an annual or perennial herb of Lycopersicon in Solanaceae. Tomato is the world's largest vegetable crop. In 2020, the global planting area exceeded 5 million hectares, the output was about 187 million tons, and the output value exceeded $100 billion. Soluble solids content (SSC) is an important component of tomato fruit quality, and directly affects consumer preferences and the economic value of processing tomatoes. The sugar content in tomato fruit accounts for about 55-65% of the total soluble solids, mainly including fructose and glucose. Existing studies have shown that, there is a serious negative correlation between fruit weight and sugar content. Selection of larger fruits often means the loss of high-sugar alleles, which directly leads to the generally lower SSC in modern large-fruited tomato varieties, making the sugar content of modern commercial tomato varieties basically less satisfactory. Therefore, how to create sweeter tomatoes without sacrificing fruit weight is an urgent problem that modern breeders need to solve.

Sugar accumulation in tomato fruit is a complex biological process, which is the result of the combined effects of multiple endogenous plant signals and the external environment. Key regulatory genes need to be further discovered.

### SUMMARY

In view of this, the present disclosure provides a polynucleotide, protein, biological material and use thereof in the improvement of the quality of plant fruits. The present disclosure achieves the regulation of the content of soluble solids, glucose, fructose, citric acid, and malic acid in plant fruits, as well as the regulation of fruit weight, by regulating the protein function and expression levels of CDPK and/or SuSy3, thereby improving the quality and flavor of tomato fruits.

In order to achieve the above object of the present disclosure, the present disclosure provides the following technical solutions.

The Present Disclosure Provides a Polynucleotide Including at Least One of the Following Nucleotide Sequences:
(11) a sequence as set forth in any one of SEQ ID NOs: 3 and 23-36;
(12) a complementary sequence, degenerate sequence or homologous sequence of any one of the sequences set forth in SEQ ID NOs: 3 and 23-36, where the homologous sequence is a sequence having at least 75% identity to any one of the sequences set forth in SEQ ID NOs: 3 and 23-36;
(13) a sequence that hybridizes to any of the sequences set forth in SEQ ID NOs: 3 and 23-36 under stringent conditions, or a complementary sequence thereof;
(14) a cDNA sequence of any one of the sequences described in the above items (11) to (13);
(15) a sequence as set forth in any one of SEQ ID NOs: 11 and 95-108;
(16) a complementary sequence, degenerate sequence or homologous sequence of any one of the sequences set forth in SEQ ID NOs: 11, and 95-108, where the homologous sequence is a sequence having at least 75% identity to any one of the sequences set forth in SEQ ID NOs: 11 and 95-108;
(17) a sequence that hybridizes to any one of the sequences set forth in SEQ ID NOs: 11 and 95-108 under stringent conditions, or a complementary sequence thereof; and
(18) a cDNA sequence of any one of the sequences described in the above items (15) to (17).

CDPKs are a class of calcium-dependent protein kinases that regulate the expression of downstream target genes mainly through phosphorylation.

In particular embodiments provided herein, CDPK is CDPK8 and/or CDPK9. CDPK8 gene sequence is a sequence described in the above items (11)-(14); CDPK9 gene sequence is a sequence described in the above items (15) (18).

SuSy3 is a glycosyltransferase that is widely involved in multiple metabolic processes in plants, including the transport and distribution of sucrose, starch synthesis, cellulose synthesis and cell wall composition, biotic and abiotic adversities, etc. The present disclosure discovered a calcium-dependent protein kinase 8 (SICDPK8), which is mainly expressed in mature fruits with fixed fruit size. The present disclosure found that, CDPK8 protein can interact with SuSy3 protein to regulate the stability of SuSy3 protein by phosphorylating it, thereby affecting the sugar content of tomato fruit. In addition, the present disclosure also found that, CDPK9 also has the above functions.

In particular embodiments provided herein, CDS sequence of CDPK8 includes at least one of the following sequences:
(1) a sequence as set forth in any one of SEQ ID NOs: 1, and 37-65;
(2) a complementary sequence, degenerate sequence or homologous sequence of any one of the sequences set forth in SEQ ID NOs: 1 and 37-65, where a homologous sequence is a sequence having at least 75% identity to any one of the sequences set forth in SEQ ID NOs: 1 and 37-65;
(3) a sequence that hybridizes to any one of SEQ ID NOs: 1 and 37-65 under stringent conditions or a complementary sequence thereof; and
(4) a cDNA sequence of any one of sequences described in the above items (1) to (3).

In particular embodiments provided herein, CDS sequence of CDPK9 includes at least one of the following sequences:
(1) a sequence as set forth in any one of SEQ ID NOs: 9 and 109-152;
(2) a complementary sequence, degenerate sequence or homologous sequence of any one of the sequences set forth in SEQ ID NOs: 9 and 109-152, where a homologous sequence is a sequence having at least 75% identity to any one of the sequences set forth in SEQ ID NOs: 9 and 109-152;
(3) a sequence that hybridizes to any one of the sequences set forth in SEQ ID NOs: 9 and 109-152 under stringent conditions, or a complementary sequence thereof; and
(4) a cDNA sequence of any one of sequences described in the above items (1) to (3).

Further, a homologous sequence is a nucleotide sequence having about at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity with original nucleotide sequence, or a corresponding cDNA molecule thereof.

In the present disclosure, a homologous sequence has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity with original nucleotide sequence and has the same function, all of which are within the protection scope of the present disclosure.

In some embodiments, a homologous sequence has 75-85%, 76-86%, 77-87%, 78-88%, 79-89%, 80-90%, 81-91%, 82-92%, 83-93%, 84-94%, 85-95%, 86-96%, 87-97%, 88-98%, 89-99%, 90-95%, 91-96%, 92-97%, 93-98%, 94-99%, 95-100%, 85-90%, 86-91%, 87-92%, 88-93% or 89-94% identity with original nucleotide sequence and has the same function, all of which are within the protection scope of the present disclosure.

Furthermore, the homologous sequence encodes exactly the same protein conserved domain as the original nucleotide sequence. Particularly, the conserved structural domain of the protein is shown in FIG. 1A and FIG. 1B.

Particularly, a nucleotide sequence molecule may be DNA such as cDNA, genomic DNA or recombinant DNA; a nucleotide sequence molecule may also be RNA, such as mRNA or hnRNA.

The nucleotide sequences of CDPK8 and/or CDPK9 are not necessarily completely the same in different tomato materials. The present disclosure provides nucleotide sequences of CDPK8 and/or CDPK9 in other tomato materials obtained during the research process, where the genome identity of CDPK8 among different varieties is 98.734-100%, and the CDS identity is 92.83-100% (see Table 1 for details), where the sequences that are not completely the same as SEQ ID NO: 3 are set forth in SEQ ID NOs: 23-36, and the sequences that are not completely the same as SEQ ID NO: 1 are set forth in SEQ ID NOs: 37-65; the genome identity of CDPK9 among different varieties is 98.734-100%, and the CDS identity is 90.59-100% (see Table 2 for details), where the sequences that are not completely the same as SEQ ID NO: 11 are set forth in SEQ ID NOs: 95-108, and the sequences that are not completely the same as SEQ ID NO: 9 are set forth in SEQ ID NOs: 109-152.

The present disclosure further provides a protein, which is at least one of the following sequences:
(21) a sequence as set forth in any one of SEQ ID NOs: 2 and 66-94;
(22) a fusion protein obtained by connecting a tag to N-terminus and/or C-terminus of any of the sequences set forth in SEQ ID NOs: 2 and 66-94;
(23) a protein having the same function obtained by substituting and/or deleting and/or adding one or more amino acid residues in any of the sequences set forth in SEQ ID NOs: 2 and 66-94;
(24) a protein having at least 75% identity to any one of the sequences set forth in SEQ ID NOs: 2 and 66-94 and having the same function;
(25) any one of the sequences set forth in SEQ ID NOs: 10 and 153-196;
(26) a fusion protein obtained by connecting a tag to the N-terminus and/or C-terminus of any of the sequences set forth in SEQ ID NOs: 10 and 153-196;
(27) a protein having the same function obtained by substituting and/or deleting and/or adding one or more amino acid residues in any of the sequences set forth in SEQ ID NOs: 10 and 153-196; and
(28) a protein having at least 75% identity with any one of the sequences set forth in SEQ ID NOs: 10 and 153-196 and having the same function.

In particular embodiments provided herein, CDPK is CDPK8 and/or CDPK9. The CDPK8 protein sequence is a sequence described in the above items (21)-(24); the CDPK9 protein sequence is a sequence described in the above items (25)-(28).

Particularly, a protein with the same function obtained by substitution and/or deletion and/or addition of one or several amino acid residues refers to a protein with exactly the same protein conserved domain. More particularly, the conserved domain of the protein is shown in FIG. 1A and FIG. 1B.

Furthermore, a homologous sequence may also be a protein sequence having about at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with original protein sequence.

In the present disclosure, a homologous sequence may also have at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity with original protein sequence and has the same function, all of which are within the protection scope of the present disclosure.

In some embodiments, the homologous sequence may also have 75-85%, 76-86%, 77-87%, 78-88%, 79-89%, 80-90%, 81-91%, 82-92%, 83-93%, 84-94%, 85-95%, 86-96%, 87-97%, 88-98%, 89-99%, 90-95%, 91-96%, 92-97%, 93-98%, 94-99%, 95-100%, 85-90%, 86-91%, 87-92%, 88-93% or 89-94% identity with original protein sequence and has the same function, all of which are within the protection scope of the present disclosure.

Furthermore, the homologous sequence has exactly the same protein conserved domain as original protein sequence. Particularly, the conserved structural domain of the protein is shown in FIG. 1A and FIG. 1B.

The protein sequences of CDPK8 and/or CDPK9 are not necessarily completely the same in different tomato materials. The present disclosure provides protein sequences of CDPK8 and/or CDPK9 in other tomato materials obtained during the research process, where the identity of the CDPK8 protein sequences between different varieties is 93.007-100% (see Table 1 for details), where the sequences that are not completely the same as SEQ ID NO: 2 are set forth in SEQ ID NOs: 66-94; the identity of the CDPK9 protein sequences between different varieties is 90.037-100% (see Table 2 for details), where the sequences that are not completely the same as SEQ ID NO: 10 are set forth in SEQ ID NOs: 153-196.

The present disclosure further provides a biomaterial, which is any one of the following (31) to (35):
(31) a knockout cassette for knocking out the aforementioned polynucleotide;
(32) a knockout vector for knocking out the aforementioned polynucleotide;
(33) a recombinant microorganism for knocking out the aforementioned polynucleotide;
(34) a plant cell line for knocking out the aforementioned polynucleotide; and
(35) a plant protoplast, cell or callus tissue in which any one of the above items (31) to (33) is introduced.

The present disclosure further provides use of any one of the above polynucleotide, protein and biological material in the improvement of the quality of plant fruits.

In the embodiments provided herein, the plant is a plant with edible parts such as fruits, roots, tubers, etc.

Preferably, the plants are berry plants, Solanaceae plants, Rosaceae plants, Rutaceae plants, Convolvulaceae plants, Dioscorea plants, Cucurbitaceae plants, etc., but are not limited to these plant species. As long as the plant species are recognized by those skilled in the art, they are within the protection scope of the present disclosure.

In the embodiments provided herein, berry plants include but are not limited to: tomato, grape, kiwi, papaya, pomegranate, ginseng fruit, blueberry, persimmon, passion fruit, dragon fruit, wax apple, and the like.

In the embodiments provided herein, Solanaceae plants include but are not limited to: potato, eggplant, tomato, pepper, wolfberry, and the like.

In the embodiments provided herein, Rosaceae plants include but are not limited to: pear, apple, peach, apricot, cherry, plum, prune, crabapple, loquat, hawthorn, strawberry, raspberry and the like.

In the embodiments provided herein, Rutaceae plants include but are not limited to: grapefruit, phellodendron, lemon, orange, mandarin orange, tangerine, and the like.

In particular embodiments provided herein, the plant is tomato.

In particular embodiments provided herein, the improvement of the quality of plant fruits includes increasing the content of soluble solids, sugar, and citric acid in the plant fruits, reducing the content of malic acid, and maintaining the weight of the fruits.

In particular embodiments provided herein, the plant is tomato, and the contents of soluble solids, sugar, citric acid, malic acid, and the weight of the tomato fruit are measured at the red ripe stage of the fruit.

In the embodiments provided herein, the sugar includes one or more of glucose, fructose, and sucrose.

In particular embodiments provided herein, the sugar includes glucose and/or fructose.

In some embodiments, the content of soluble solids, sugar or citric acid in the fruit of the plant of interest is at least 30% higher than that of the recipient plant. In some embodiments, the content of soluble solids, sugar or citric acid in the fruit of the plant of interest is at least 10%, at least 20%, at least 30%, at least 40%, at least 50%, at least 60%, at least 70%, at least 80%, at least 90%, or at least 100% higher than that of the recipient plant.

In some embodiments, the malic acid content in the fruit of the plant of interest is at least 30% lower than that of the recipient plant. In some embodiments, the malic acid content in the plant fruit of the target plant is at least 10%, at least 20%, at least 30%, at least 40%, at least 50%, at least 60%, at least 70%, at least 80%, at least 90%, or at least 100% lower than that of the recipient plant.

The present disclosure further provides use of a polynucleotide, protein, and biological material in the improvement of the quality of a plant fruit, where the phosphorylation level of SuSy3 protein is reduced by at least one of the following methods:
(1) inhibiting the expression of CDPK or inactivating the phosphorylase of CDPK; the polynucleotide of CDPK gene is the aforementioned polynucleotide, or CDPK protein is the aforementioned protein;
(2) mutating one or more amino acids in SuSy3 protein, where the mutation site is a phosphorylation site mediated by CDPK, without altering the glycosyltransferase activity of the SuSy3 protein; and
(3) specifically overexpressing the SuSy3 protein in the fruit.

Particularly, the present disclosure can control the phosphorylation level of SuSy3 protein and the quality of tomato fruit and improve the flavor of tomato fruit by the following three methods.

### 1. Functional Regulation of CDPK

The present disclosure first discovered that, CDPK8/CDPK9-SuSy3 interaction regulates sugar accumulation in tomato fruit. The functional loss of CDPK8 and CDPK9 can promote the accumulation of glucose and fructose in tomato fruit, and increase SSC. This gene function is reported for the first time in tomatoes, and this gene is an important gene for sugar accumulation in tomatoes. The protein stability of SuSy3 protein is regulated by CDPK8 and CDPK9, and is also involved in the biological process of tomato sugar accumulation. In present disclosure, the sugar content and SSC of tomatoes and the weight of fruit can be controlled by regulating the protein functions and expression levels of CDPK8, CDPK9, and SuSy3.

The present disclosure identified two key genes CDPK8 and CDPK9 that could regulate SSC of tomato fruit, and utilized gene editing and other technologies to regulate the gene expression and protein function of CDPK8 and CDPK9 by inserting a 12 bp sequence in the promoter region and mutating the key kinase domain in the coding region, thereby creating a new tomato breeding material with increased SSC, glucose, fructose, and citric acid contents in the fruit, decreased malic acid content, and no significant change in single fruit weight.

By regulating the function of CDPK8, when CDPK8 function is lost, SuSy3 protein is not phosphorylated, and the stability of the protein can be maintained to function normally in the sugar synthesis pathway. Therefore, sweet tomatoes can be created without sacrificing fruit weight, and their glucose and fructose content can be increased by 30%.

CDPK9 and CDPK8 are homologous genes, and CDPK9 protein can also interact with SuSy3 protein to phosphorylate SuSy3. When CDPK8 and its homologous gene CDPK9 mutate at the same time, an additive effect can be produced, further expanding the impact of CDPK8 on sugar content and fruit sweetness.

### 2. Mutation of SuSy3

CDPK can phosphorylate the serine at position 11 (S11) of SuSy3 protein. When the serine at position 11 mutates into other amino acid, it can prevent CDPK8 from phosphorylating SuSy3. SuSy3 protein is not phosphorylated, which maintains the stability of the protein and allows it to function normally in the sugar synthesis pathway, thus creating sweet tomatoes without sacrificing fruit weight.

### 3. Specific Overexpression of SuSy3 Protein in Fruit

The present disclosure inserts a promoter E8 for specific expression in tomato fruits, thereby causing the SuSy3 protein to be specifically overexpressed in the fruit. Even though CDPK can phosphorylate part of the SuSy3 protein and cause degradation, a large amount of SuSy3 protein with normal function still exists in the tomato fruit, thereby playing a normal role in the sugar synthesis pathway. Therefore, sweet tomatoes can be created without sacrificing the weight of the fruit.

In particular embodiments provided herein, the genome sequence of SuSy3 includes at least one of the following sequences:
(1) a sequence as set forth in SEQ ID NO: 19;
(2) a complementary sequence, degenerate sequence or homologous sequence of the sequence set forth in SEQ ID NO: 19, where a homologous sequence is a sequence having at least 75% identity with SEQ ID NO: 19;
(3) a sequence that hybridizes to the sequence set forth in SEQ ID NO: 19 under stringent conditions or a complementary sequence thereof; and
(4) a cDNA sequence of any one of sequences in the above items (1) to (3).

In particular embodiments provided herein, CDS sequence of SuSy3 includes at least one of the following sequences:
(1) a sequence as set forth in SEQ ID NO: 17;
(2) a complementary sequence, degenerate sequence or homologous sequence of the sequence set forth in SEQ ID NO: 17, where a homologous sequence is a sequence having at least 75% identity with SEQ ID NO: 17;
(3) a sequence that hybridizes to the sequence set forth in SEQ ID NO: 17 under stringent conditions or a complementary sequence thereof; and
(4) a cDNA sequence of any one of sequences in the above items (1) to (3).

In particular embodiments provided herein, the protein sequence of SuSy3 includes at least one of the following sequences:
(1) a sequence as set forth in SEQ ID NO: 18;
(2) a fusion protein obtained by connecting a tag to N-terminus and/or C-terminus of the sequence set forth in SEQ ID NO: 18;
(3) a protein having the same function obtained by substituting and/or deleting and/or adding one or more amino acid residues in the sequence set forth in SEQ ID NO: 18; and
(4) a protein having at least 75% identity with the sequence set forth in SEQ ID NO: 18 and having the same function.

In particular embodiments provided herein, in the above first method, the method for inhibiting the expression of CDPK or inactivating the phosphorylase of CDPK includes regulating a promoter and/or changing a coding region.

In particular embodiments provided herein, the method for inhibiting the expression of CDPK or inactivating the phosphorylase of CDPK is to regulate the promoter, and the specific method for regulating the promoter is: inserting the sequence as set forth in SEQ ID NO: 22 into the CDPK promoter region, for example, inserting into the CAACA conserved site region. However, the insertion sequence is not limited thereto, as long as it can achieve the technical effect of inhibiting CDPK expression.

Through analysis of the CDPK8 genome sequence, it was found that there is a 12 bp sequence variation in the promoter region (TTGTTGTTGTTG, as set forth in SEQ ID NO: 22), which is closely related to the SSC of tomato fruit. The Indel sequence can inhibit gene expression by recruiting the RAV transcription repressor. The research results showed that, SSC of tomato materials containing the 12 bp Indel sequence is significantly higher than that of tomato materials lacking the 12 bp Indel sequence, indicating that the 12 bp Indel sequence variation is a key site for regulating the SSC of tomato fruit.

In particular embodiments provided herein, the method for inhibiting the expression of CDPK or inactivating the phosphorylase of CDPK is to change the coding region, and changing the coding region particularly includes: deleting, inserting and/or mutating (non-synonymous mutation) a key kinase domain of the coding region.

In the particular embodiments provided herein, changing the coding region particularly includes: deleting an ATP binding domain, or changing the coding region to terminate protein translation prematurely.

In particular embodiments provided herein, the key kinase domain of the coding region includes leucine at position 62 (L62) and glycine at position 63 (G63) of CDPK8, and leucine at position 64 (L64) and glycine at position 65 (G65) of CDPK9. However, the key functional domain is not limited thereto, as long as the technical effect of inhibiting CDPK expression can be achieved.

Particularly, the full length of CDPK8 contains 533 amino acids. The results of this study showed that, the leucine at position 62 (L62) and the glycine at position 63 (G63) of CDPK8 are essential for the catalytic activity of CDPK8 phosphorylase. When L62 and G63 are missing, the activity of CDPK8 phosphorylase will be lost through the loss of ATP binding domain or premature termination of protein translation, thus it will be unable to phosphorylate SuSy3 protein.

Particularly, the full length of CDPK9 contains 533 amino acids. The results of this study showed that, the leucine at position 64 (L64) and the glycine at position 65 (G65) of CDPK9 are essential for the catalytic activity of CDPK9 phosphatase. When L64 and G65 are missing, the activity of CDPK9 phosphatase will be lost through the loss of ATP binding domain or premature termination of protein translation, thus it will be unable to phosphorylate SuSy3 protein.

In particular embodiments provided herein, the method for inhibiting the expression of CDPK or inactivating the phosphorylase of CDPK includes at least one of: CRISPR/Cas9 gene editing technology, promoter editing technology, TALEN technology, T-DNA insertion, EMS mutagenesis, or ZFN technology.

In particular embodiments provided herein, in the above second method, the mutation site is serine at position 11 of the SuSy3 protein.

In particular embodiments provided herein, in the above second method, the serine at position 11 of the SuSy3 protein is mutated to alanine.

In particular embodiments provided herein, in the above third method, the promoter for specifically overexpressing the SuSy3 protein in the fruit is the E8 promoter.

In the embodiments provided herein, the tomato varieties include but are not limited to: SV7845TH, SVTH4018, Antelaisi, SV4224TH, SV8795TG, SV3026TG, Meishuai, SV0313TG, Hongsui, Diliya, Deruisi, Fengege, Oushuo, Huifu, SVTH1366, Ouke, Meibei, BOLZANO, DRC 564, DRK 936, TOMIMARU MUCHOO), DELISHER, Strabena, DR0607TC, Anbeisi, Jiuli, Camry, Winter warm, Sibeide, Kevin, Jiuli 200, Tiannaxi No. 3, Ruifei No. 5, Ruifei No. 2, Ameizi No. 5, PF207008, Juli, Beiying, Zidali, Ruifei, Kaisa, Ono, Fenbeiying, Rabi, 72-CH0353, Fengshou 74-560, Zulfia (73-610), Florantino (72-163), Fulltom (72-729), Forticia (72-719), Glorioso (74-112), Cappricia (72-466), Brioso (72-130), Kawaguchi (72-541), Kivu (72-629), Tatami (72-175), Tarffy 6917 (76-IM6917), Genery (72-192), Moscatel (74-104), Siberite 915 (73-915), Siberite 916 (73-916), Plumola (72-001), Messina (73-47), Paulanca (72-534), Logure (73-571), Santina (72-763), Operino (72-187), Ternetto (72-190), Tomary (72-191), Ruifen 882 (73-882), TY 12, Beril (73-14), Abellus (73-583), Fortesa (72-152), Lidertom (74-254), Solarino (72-150), Vacetto (72-164), Reddery (72-008), Endeavour (72-487), Suncery (74-132), Gourami (72-021), Betty (72-126) Pareso, Hibachi (72-241), Aruru (72-193), Lauster, Magino (72-195), Yingla, Gutian, Solana, Andara, Andolina, Keren, Summer Sunshine, Honglilai, Diruisen, Qiulian, Nicola, Saint Laurent, Fendi, Dali, Hanyu 522, Ainila, Babaoli, Lasaier, Jinlong, Walter, Kailong, Dorothy, Fulaixin, Raphael, Haiyue, Luola, Annecy, Rila, Sidi, Fola 3661, Shiluoqi, Davidson, Jinpeng No. 8, Jinpeng 101, Jinpeng 950, Jinpeng 2095, Jinpeng Sengbiya 45, Jinpeng Sengbiya 50, Jinpeng No. 8 B, Jinpeng No. 9, Jinpeng No. 10, Jinpeng 148, Jinpeng 152, Jinpeng 218, Jinpeng 261, Jinpeng 322, Jinpeng 945, Jinpeng 951, Jinpeng 1186, Jinpeng 1198, Jinpeng Qiusheng, Jinpeng Rongwei, Jinpeng Tianyi No. 2, Jinpeng 102, Jinpeng 1828, Jinpeng M708, Jinpeng M5038, Jinpeng No. 11, Jinpeng 12-2, Jinpeng 18-98, Jinpeng 236, Jinpeng 1729, Jinpeng Black Marshal, Jinpeng Sengbiya 04, Jinpeng 11-21, Jinpeng 1521, Jinpeng 1605, Jinpeng M6, Jinpeng M7, Jinpeng M158, Jinpeng No. 5, Jinpeng Xianai, Jinpeng No. 1, Jinpeng 128, Jinpeng M215, Jinpeng 513, Jinpeng 515, Jinpeng No. 6, Jinpeng No. 3, Jinpeng Red String 132, Jinpeng Pink Girl No. 2, Jinpeng Little Yellow Crown, Jinpeng Pink Girl No. 1, Jinpeng Little Yellow Crown 58, Jinpeng No. 19, Jinpeng No. 44, Jinpeng No. 53, Jinpeng No. 75, Jinpeng 151, Jinpeng Show Girl, Holy Banquet 1523, Xilaide No. 1, Darwin, Bora, Ferrari, Osina, Olina, Nunhem 1618, Nunhem 1718, Praise No. 2, Holy Banquet 3767, Blooming Red 6415, Jin Xiaoling, Pink Girl, Pink Girl No. 2, Lifei, Pink Taro No. 3, Delicious 925, Purple Taro, Athena, Show Taro, Qing Taro, Pink Lide, Pink Lide No. 2, Red Rise, Red Rise No. 2, Beautiful 601, Yellow Crown 16, Yellow Crown No. 2, Yellow Crown No. 3, Golden Pear, Yellow Little Y, Black Pearl No. 2, Guyu Tianfei® F1, Guyu Tianfei® No. 2, Guyu Tianfei® No. 3, Guyu Tianfei® No. 6 F1, Guyu Tianfei® No. 7, Guyu Tianfei® No. 8, Guyu Tianfei® No. 9, Guyu Tianfei® No. 10, Guyu Tianfei® No. 11, Guyu Zhuofen®227, Guyu Zhuofen® No. 1, Guyu Zhuofen® No. 2, Guyu Zhuofen® No. 3, Guyu Zhuofen® No. 5, Guyu z Tianci No. 1, Guyu z Tianci No. 2, Guyu z Tianci No. 3, Guyu z Tianci No. 4, Guyu z Tianci No. 5, Guyu z Tianci No. 6, Guyu Tianci 595, Guyu Tianci 585, Guyu Tianci 575, Guyu Tianci 565F1, Guyu Tianqi No. 1, Guyu Tianqi No. 2, Guyu Tianqi No. 3, Guyu Zhuohang® No. 1, Guyu Zhuohang® No. 2, Guyu Zhuohang® No. 3, Guyu Zhuohang® No. 4, Red Kerry No. 1, Guyu Tianmi No. 1, Guyu Tianmi No. 2, Guyu Tianmi No. 3, Hehong No. 1 Tomato, Hehong No. 2 Tomato, Hehong No. 3 Tomato, Yingjiahe No. 3, Yingjiahe No. 2, Yingjiahe No. 5, Yingjiahe No. 4, Yingjiahe No. 1, Pengbo No. 3, Pengbo No. 2, Pengbo No. 1, Herunsheng No. 4, Herunsheng No. 3, Herunsheng No. 2, Herunsheng No. 1, Baoliyuan No. 6, Baoliyuan No. 5, Baoliyuan No. 4, Baoliyuan No. 3, Baoliyuan No. 2, Baoliyuan No. 1, Millennium, Fengzhu, Xiaoxia, Chuntao, Guanghui 101, Money maker, M82, Ailsa Craig, Provence, Ruixing Dabao, Pink Baby, Zhongshu No. 4, Zhongshu No. 5, Busan 88, Pink Taro, etc.

In some embodiments, the tomato is selected from the group consisting of: Money maker, M82, Ailsa Craig, Millennium, Provence, Ruixing Dabao, Zidali, Pink Baby, Summer Sunshine, Luola, Zhongshu No. 4, Zhongshu No. 5, Busan 88, Pink Taro, Antelaisi, Rabi, Lauster, Jinpeng No. 8, Jinpeng Little Yellow Crown, Guyu Tianci® No. 2, and Chuntao.

The present disclosure further provides a plant or a related product thereof, where the plant is one of the following plants:
(1) a plant obtained by using the aforementioned biological material, or a plant obtained by using the aforementioned method;
(2) a progeny formed by self-pollination of the plant described in the above item (1), and a plant grown from the progeny; and
(3) a progeny formed by hybridization of the plant described in the above item (1) with other varieties, and a plant grown from the progeny.

In particular embodiments provided herein, the related product is a plant part, plant cell, tissue culture, protoplast, fruit, pollen or seed.

The present disclosure further provides a commercial plant product, which is a commercial plant product made from the above plant or a related product thereof; the commercial plant product includes food, medicine, cosmetics/skin care product, feed or other products.

In the particular embodiments provided herein, the food includes: fresh fruit, dried fruit, frozen fruit, pickled fruit, candied fruit, fried fruit or various processed forms of fruit; the various processed forms of fruit include fruit strip, fruit block, fruit slice, fruit particle, fruit powder, fruit paste, fruit juice, or fermented product.

The medicine includes: various dosage forms made from medicinal parts of the plant.

The cosmetics/skin care product includes: facial cleanser, toner, lotion, face cream, essence, facial mask, eye cream, eye mask, primer lotion, sunscreen, liquid foundation, or BB cream.

The feed includes: liquid feed, solid feed, semi-solid feed, or feed raw material.

The present disclosure further provides a method for producing a commercial plant product, which includes obtaining the above plant or a related product thereof to produce the commercial plant product.

The present disclosure further provides a tomato plant or seed capable of producing tomato fruit at mature, the tomato fruit having an average total soluble solids content (SSC) of at least 4.0 and an average fresh fruit weight of at least 80 g.

The present disclosure further provides a tomato plant or seed capable of producing tomato fruit at maturity, the tomato fruit having an average glucose level of at least 10 mg/g FW and an average fresh fruit weight of at least 80 g.

The present disclosure further provides a tomato plant or seed capable of producing tomato fruit at maturity, the tomato fruit having an average fructose level of at least 10 mg/g FW and an average fresh fruit weight of at least 80 g.

Preferably, the tomato plant or seed has a genetic background substantially derived from any one of the above-mentioned tomato varieties.

The present disclosure further provides an improved tomato plant or seed including one or more genetic modifications and is capable of producing tomato fruits at maturity, and it includes:
(i) an average total soluble solids content (SSC) which is at least 5% higher than that of a control tomato plant or seed, preferably at least 8%, 10%, 12%, 15%, 18%, 20%, 22%, 25%, 28%, 30%, 32%, 35%, 38% or 40% higher; and
(ii) an average fresh fruit weight substantially similar to that of a control tomato plant and seed;
where both the control tomato plant or seed and the modified tomato plant or seed include substantially the same genetic background, except for the one or more genetic modifications.

The present disclosure further provides an improved tomato plant or seed, where it includes one or more genetic modifications and is capable of producing tomato fruits at maturity, and it includes:
(i) an average glucose level which is at least 5% higher than that of a control tomato plant or seed, preferably at least 8%, 10%, 12%, 15%, 18%, 20%, 22%, 25%, 28%, 30%, 32%, 35%, 38% or 40% higher; and
(ii) an average fresh fruit weight substantially similar to that of a control tomato plant and seed;
where both the control tomato plant or seed and the modified tomato plant or seed include substantially the same genetic background, except for the one or more genetic modifications.

The present disclosure further provides an improved tomato plant or seed, where it includes one or more genetic modifications and is capable of producing tomato fruits at maturity, and it includes:
(i) an average fructose level which is at least 5% higher than that of a control tomato plant or seed, preferably at least 8%, 10%, 12%, 15%, 18%, 20%, 22%, 25%, 28%, 30%, 32%, 35%, 38% or 40% higher; and
(ii) an average fresh fruit weight substantially similar to that of a control tomato plant and seed;
where both the control tomato plant or seed and the modified tomato plant or seed include substantially the same genetic background, except for the one or more genetic modifications.

Preferably, the one or more genetic modifications are located in a gene selected from the group consisting of: CDPK8, CDPK9, SuSy3, and a homolog thereof.

Preferably, the one or more genetic modifications regulate the expression or activity of one or more genes selected from the group consisting of: CDPK8, CDPK9, SuSy3, and a homolog thereof.

Preferably, the one or more genetic modifications include a transgene targeting a gene selected from the group consisting of: CDPK8, CDPK9, SuSy3, and a homologue thereof.

In particular embodiments provided herein, the one or more genetic modifications are in a gene of any one of SEQ ID NOs: 3, 11, 23-36, and 95-108.

In particular embodiments provided herein, the one or more genetic modifications result in the prevention or regulation of phosphorylation of SuSy3 or a homologue thereof.

In particular embodiments provided herein, the one or more genetic modifications result in the prevention or regulation of serine phosphorylation at position 11 amino acid residue of SuSy3.

In particular embodiments provided herein, the one or more genetic modifications result in delayed degradation of SuSy3 or a homolog thereof.

In particular embodiments provided herein, the one or more genetic modifications result in over-accumulation of SuSy3 or a homolog thereof.

In particular embodiments provided herein, the one or more genetic modifications include CDPK8 mutation, CDPK9 mutation, and double mutation of CDPK8 and CDPK9.

In particular embodiments provided herein, the one or more genetic modifications include a CDPK8 deletion mutation, a CDPK9 deletion mutation, and a double deletion mutation of CDPK8 and CDPK9.

In particular embodiments provided herein, the one or more genetic modifications include a 12 bp (a sequence set forth in SEQ ID NO: 22) insertion in the CDPK8 promoter present in Hap2.

The present disclosure further provides a breeding method for improving plant fruit quality, where it includes using a molecular marker to assist in plant breeding, where the molecular marker is a molecular marker for detecting presence or absence of CDPK8 and/or CDPK9.

This disclosure detected different tomato materials and found that, there are multiple mutation sites in high-sugar tomato varieties (as shown in the table 0 below). These mutation sites are closely linked to the above-mentioned 12 bp key mutation site, and can be used to screen for high-sugar content phenotypes.

In particular embodiments provided herein, the molecular markers for detecting presence or absence of CDPK8 include at least one of the following:

TABLE 10

| | | | | Position | |
|---|---|---|---|---|---|
| | | | | relative | |
| | | | | to the | |
| S12.50 | | | | starting | |
| ch11 | Reference | Alternative | Region | codon ATG | Detail |
| 51186147 | A | C | Downstream | +9460 bp | Most significant |
| | | | | | mutation |
| 51188895 | G | C | Downstream | +6712 bp | Mutation |
| 51188914 | A | G | Downstream | +6693 bp | Mutation |
| 51189060 | C | T | Downstream | +6547 bp | Mutation |
| 51189072 | T | C | Downstream | +6535 bp | Mutation |
| 51189139 | G | A | Downstream | +6468 bp | Mutation |
| 51189300 | G | T | Downstream | +6307 bp | Mutation |
| 51189382 | C | T | Downstream | +6225 bp | Mutation |
| 51189503 | C | T | Downstream | +6104 bp | Mutation |
| 51189521 | G | A | Downstream | +6086 bp | Mutation |
| 51189733 | A | - | Downstream | +5874 bp | Deletion |
| 51189896 | C | G | Downstream | +5711 bp | Mutation |
| 51189975 | - | T | Downstream | +5632 bp | Insertion |
| 51190011- | CATT | C--T/CA-- | Downstream | +5596 bp/ | Deletion/ |
| 51190014 | | /TA-T | | +5593 bp | Mutation |
| 51190111 | A | - | Downstream | +5496 bp | Deletion |
| 51190132 | T | C | Downstream | +5475 bp | Mutation |
| 51190458 | G | A | Downstream | +5149 bp | Mutation |
| 51190546 | G | A | Downstream | +5061 bp | Mutation |
| 51191042 | C | T | Intron | +4565 bp | Mutation |
| 51191171 | G | A | Intron | +4436 bp | Mutation |
| 51191427- | AAGTCCC | ------- | Intron | +4180 bp/ | Deletion |
| 51191440 | AAGCCCC | ------- | | +4167 bp | |
| 51191864 | G | A | Intron | +3743 bp | Mutation |
| 51191926 | A | G | Intron | +3681 bp | Mutation |
| 51192112 | A | G | Exon | +3495 bp | Synonymous |
| | | | | | mutation |
| 51192245 | A | C | Intron | +3362 bp | Mutation |
| 51193364 | C | A | Intron | +2243 bp | Mutation |
| 51193567 | G | C | Exon | +2040 bp | Synonymous |
| | | | | | mutation |
| 51193714 | G | A | Exon | +1893 bp | Non-synonymous |
| | | | | | mutation |
| | | | | | T194I |
| 51194369 | T | A | Intron | +1238 bp | Mutation |
| 51194756 | G | A | Intron | +851 bp | Mutation |
| 51194964 | C | T | Intron | +643 bp | Mutation |
| 51195083 | T | C | Intron | +524 bp | Mutation |
| 51195344 | T | C | Exon | +263 bp | Synonymous |
| | | | | | mutation |
| 51195539 | A | C | Exon | +68 bp | Synonymous |
| | | | | | mutation |
| 51195552- | TTC | --- | Exon | +55 bp/ | In-frame deletion |
| 51195554 | | | | +53 bp | mutation K18- |
| 51195789 | T | - | Promoter | −182 bp | Deletion |
| 51195816 | - | A | Promoter | −209 bp | Insertion |
| 51195897 | G | A | Promoter | −290 bp | Mutation |
| 51196183 | C | A | Promoter | −576 bp | Mutation |
| 51196264 | T | - | Promoter | −657 bp | Deletion |
| 51196441 | G | A | Promoter | −834 bp | Mutation |
| 51196462 | G | A | Promoter | −855 bp | Mutation |
| 51196483 | T | C | Promoter | −876 bp | Mutation |
| 51196878- | ATG | --- | Promoter | −1271 bp/ | Deletion |
| 51196880 | | | | −1273 bp | |
| 51196906 | T | C | Promoter | −1299 bp | Mutation |
| 51196934 | C | G | Promoter | −1327 bp | Mutation |
| 51196967 | G | A | Promoter | −1360 bp | Mutation |
| 51197003 | G | C | Promoter | −1396 bp | Mutation |
| 51197075 | T | G | Promoter | −1468 bp | Mutation |
| 51197085 | G | A | Promoter | −1478 bp | Mutation |
| 51197106 | A | T | Promoter | −1499 bp | Mutation |
| 51197121 | T | C | Promoter | −1514 bp | Mutation |
| 51197214 | A | G | Promoter | −1607 bp | Mutation |
| 51197221 | G | C | Promoter | −1614 bp | Mutation |
| 51197324 | C | T | Promoter | −1717 bp | Mutation |
| 51197357 | T | - | Promoter | −1750 bp | Deletion |
| 51197369 | G | A | Promoter | −1762 bp | Mutation |
| 51197378 | G | T | Promoter | −1771 bp | Mutation |
| 51197397 | T | A | Promoter | −1790 bp | Mutation |
| 51197421 | C | A | Promoter | −1814 bp | Mutation |
| 51197480 | G | A | Promoter | −1873 bp | Mutation |
| 51197604 | C | T | Promoter | −1997 bp | Mutation |
| 51197708 | - | AA | Promoter | −2101 bp | Insertion |
| 51197738 | G | A | Promoter | −2131 bp | Mutation |

The present disclosure further provides a gene fragment set forth in SEQ ID NO: 22, which can be inserted into the CDPK8 gene to inhibit the expression of the CDPK8 gene.

In particular embodiments provided herein, the insertion site of the gene fragment set forth in SEQ ID NO: 22 is the promoter region of the CDPK gene on chromosome 11 of tomato.

The present disclosure further provides a class of CDPK genes, where the gene ID of the CDPK gene is selected from the group consisting of: Solyc01 g006730, Solyc01 g006840, Solyc01 g008740, Solyc01 g112250, Solyc02 g032820, Solyc02 g083850, Solyc03 g031670, Solyc03 g033540, Solyc03 g113390, Solyc04 g009800, Solyc04 g049160, Solyc04 g081910, Solyc05 g056570, Solyc06 g065380, Solyc06 g073350, Solyc07 g064610, Solyc08 g008170, Solyc09 g005550, Solyc10 g074570, Solyc10 g076900, Solyc10 g079130, Solyc10 g081640, Solyc10 g081740, Solyc11 g006370, Solyc11 g018610, Solyc11 g064900, and Solyc12 g099790.

Compared with the conventional technology, the present disclosure has the following beneficial effects:
The present disclosure found that, CDPK8, CDPK9 and SuSy3 are key regulatory genes that affect the sugar content of fruits. In application, the protein functions and expression levels of CDPK8, CDPK9 and SuSy3 can be regulated to achieve the regulation of SSC, glucose, fructose, citric acid and malic acid content in plant fruits without significantly affecting the fruit weight, thereby improving the quality and flavor of tomato fruit.

### BRIEF DESCRIPTION OF THE DRAWINGS

FIG. 1A shows the prediction of the conserved domain of the protein sequence of tomato CDPK8 gene.

FIG. 1B shows the detailed information of the position of the conserved domain of tomato CDPK8 protein sequence.

FIG. 2A shows the tomato CDPK8 genome sequence information.

FIG. 2B shows that a 12 bp sequence variation can divide the natural tomato population into two haplotypes.

FIG. 2C shows the proportion of tomato materials with the Hap2 haplotype in Solamim pimpinellifolium (PIM), Lycopersicon esculentum var. cerasiforme A. Gray (CER) and BIG tomato (BIG) populations.

FIG. 3A shows cdpk8-1 genotype 1 and cdpk8-2 genotype of homozygous mutant strains of tomato CDPK8 gene.

FIG. 3B shows cdpk9-1 genotype of homozygous mutant strains of tomato CDPK9 gene.

FIG. 4A shows the SSC results of phenotypic identification of homozygous mutant strains of tomato CDPK8/CDPK9 genes.

FIG. 4B shows the glucose content results of phenotypic identification of homozygous mutant strains of tomato CDPK8/CDPK9 genes.

FIG. 4C shows the fructose content results of phenotypic identification of homozygous mutant strains of tomato CDPK8/CDPK9 genes.

FIG. 4D shows the citric acid content results of phenotypic identification of homozygous mutant strains of tomato CDPK8/CDPK9 genes.

FIG. 4E shows the malic acid content results of phenotypic identification of homozygous mutant strains of tomato CDPK8/CDPK9 genes.

FIG. 4F shows the average single fruit weight results of phenotypic identification of homozygous mutant strains of tomato CDPK8/CDPK9 genes.

FIG. 5A shows that tomato CDPK8 protein has phosphokinase activity, and can phosphorylate sucrose synthase SuSy3 protein.

FIG. 5B shows the stability of SuSy3 protein.

FIG. 6 shows the key sites for phosphorylation of SuSy3 protein by CDPK8.

FIG. 7A shows the average single fruit weight results of SuSy3 overexpression transgenic lines (35S-SuSy3-1/2/3).

FIG. 7B shows the total soluble solid content results of SuSy3 overexpression transgenic lines (35S-SuSy3-1/2/3).

FIG. 7C shows the average single fruit weight results of fruit-specific expression transgenic lines (E8-SuSy3-1/2/3).

FIG. 7D shows the total soluble solid content results of fruit-specific expression transgenic lines (E8-SuSy3-1/2/3).

### DETAILED DESCRIPTION OF THE EMBODIMENTS

The present disclosure discloses a polynucleotide, protein, biological material and use thereof in the improvement of the quality of plant fruits. Those skilled in the art can refer to the contents disclosed herein, and appropriately improve the process parameters to achieve the desired results. It should be particularly noted that, all similar substitutions and modifications are obvious to those skilled in the art, and they are deemed to be included in the present disclosure. The methods and disclosures of the present disclosure have been described through preferred embodiments. Relevant personnel can obviously modify or appropriately change and combine the methods and disclosures described herein without departing from the content, spirit and scope of the present disclosure to implement and apply the technology of the present disclosure.

The detection methods used in the following Examples are as follows.

The method for determination of SSC: more than 6 red-ripe fruits were selected from each plant, taking fresh juice from each plant, and using a portable digital display fully automatic sugar content handheld refractometer (PAL-1, ATAGO, Japan) to measure SSC at room temperature (20° C., adjusted and calibrated with distilled water before the experiment) and express it in Brix.

The method for determination of glucose, fructose, citric acid, and malic acid: six red-ripe fruits were selected from each plant, and the glucose and fructose contents were measured by using UPLC-MS/MS (ACQUITY UPLC I-Class Xevo TQ-S Micro, Waters). For detailed methods, please refer to references (R. Li, S. Sun, H. Wang, K. Wang, H. Yu, Z. Zhou, P. Xin, J. Chu, T. Zhao, H. Wang, J. Li, λ. Cui, FIS1 encodes a GA2-oxidase that regulates fruit firmness in tomato. Nature Communications 11, 5844 (2020).).

Method for determination of average single fruit weight: six red-ripe fruits were selected from each plant, using an electronic balance to weigh the weight of each fruit, and calculating the average value.

This disclosure compares the genome, CDS and protein identity of CDPK8 and CDPK9 of 46 tomato varieties.

TABLE 1
Identity of CDPK8 genome, CDS and protein

| | | | | Genome | CDS | Protein |
|---|---|---|---|---|---|---|
| No. | Gene | Query | Chromosome | identity | identity | identity |
| 1 | BGV006775 | CDPK8 | 11 | 100 | 93.36 | 100 |
| 2 | BGV006865 | CDPK8 | 11 | 99.71 | 95.10 | 93.357 |
| 3 | BGV007931 | CDPK8 | 11 | 99.964 | 100.00 | 95.105 |
| 4 | BGV007989 | CDPK8 | 11 | 100 | 95.63 | 100 |
| 5 | Brandywine | CDPK8 | 11 | 100 | 100.00 | 95.629 |
| 6 | EA00371 | CDPK8 | 11 | 100 | 100.00 | 100 |
| 7 | EA00990 | CDPK8 | 11 | 100 | 100.00 | 100 |
| 8 | Fla8924 | CDPK8 | 11 | 100 | 95.63 | 100 |
| 9 | Floradade | CDPK8 | 11 | 100 | 93.36 | 95.629 |
| 10 | LA2093 | CDPK8 | 11 | 99.241 | 99.53 | 93.357 |
| 11 | LYC1410 | CDPK8 | 11 | 100 | 95.57 | 99.65 |
| 12 | MM | CDPK8 | 11 | 100 | 100.00 | 95.629 |
| 13 | PAS014479 | CDPK8 | 11 | 99.187 | 100.00 | 100 |
| 14 | PI169588 | CDPK8 | 11 | 100 | 93.36 | 100 |
| 15 | PI303721 | CDPK8 | 11 | 99.982 | 93.36 | 93.357 |
| 16 | PP | CDPK8 | 11 | 99.187 | 92.89 | 93.357 |
| 17 | 1706 | CDPK8 | 11 | 100 | 100.00 | 93.007 |
| 18 | TS112 | CDPK8 | 11 | 100 | 100.00 | 100 |
| 19 | TS117 | CDPK8 | 11 | 99.638 | 92.83 | 93.007 |
| 20 | TS118 | CDPK8 | 11 | 100 | 99.53 | 99.65 |
| 21 | TS12 | CDPK8 | 11 | 100 | 97.67 | 97.552 |
| 22 | TS15 | CDPK8 | 11 | 100 | 95.16 | 95.28 |
| 23 | TS156 | CDPK8 | 11 | 100 | 100.00 | 100 |
| 24 | TS166 | CDPK8 | 11 | 100 | 95.63 | 95.629 |
| 25 | TS171 | CDPK8 | 11 | 100 | 97.73 | 97.727 |
| 26 | TS185 | CDPK8 | 11 | 100 | 100.00 | 100 |
| 27 | TS204 | CDPK8 | 11 | 100 | 100.00 | 100 |
| 28 | TS222 | CDPK8 | 11 | 100 | 97.73 | 97.727 |
| 29 | TS238 | CDPK8 | 11 | 100 | 100.00 | 100 |
| 30 | TS265 | CDPK8 | 11 | 98.734 | 97.26 | 97.378 |
| 31 | TS280 | CDPK8 | 11 | 100 | 97.73 | 97.727 |
| 32 | TS281 | CDPK8 | 11 | 100 | 100.00 | 100 |
| 33 | TS3 | CDPK8 | 11 | 100 | 97.73 | 97.727 |
| 34 | TS331 | CDPK8 | 11 | 100 | 99.53 | 99.65 |
| 35 | TS39 | CDPK8 | 11 | 99.187 | 95.57 | 95.455 |
| 36 | TS413 | CDPK8 | 11 | 99.187 | 93.30 | 93.182 |
| 37 | TS421 | CDPK8 | 11 | 98.734 | 100.00 | 100 |
| 38 | TS439 | CDPK8 | 11 | 100 | 100.00 | 100 |
| 39 | TS545 | CDPK8 | 11 | 99.187 | 99.53 | 99.65 |
| 40 | TS60 | CDPK8 | 11 | 100 | 95.63 | 95.629 |
| 41 | TS623 | CDPK8 | 11 | 99.187 | 99.53 | 99.65 |
| 42 | TS629 | CDPK8 | 11 | 100 | 97.73 | 97.727 |
| 43 | TS692 | CDPK8 | 11 | 100 | 100.00 | 100 |
| 44 | TS80 | CDPK8 | 11 | 98.734 | 100.00 | 100 |
| 45 | TS9 | CDPK8 | 11 | 100 | 97.73 | 97.727 |
| 46 | TS96 | CDPK8 | 11 | 100 | 99.53 | 99.65 |

TABLE 2
Identity of CDPK9 genome, CDS and protein

| | | | | Genome | CDS | Protein |
|---|---|---|---|---|---|---|
| No. | Gene | Query | Chromosome | identity | identity | identity |
| 1 | BGV006775 | CDPK9 | 1 | 100 | 97.42 | 96.168 |
| 2 | BGV006865 | CDPK9 | 1 | 99.71 | 99.82 | 98.723 |
| 3 | BGV007931 | CDPK9 | 1 | 99.964 | 97.42 | 96.168 |
| 4 | BGV007989 | CDPK9 | 1 | 100 | 97.42 | 96.168 |
| 5 | Brandywine | CDPK9 | 1 | 100 | 97.42 | 96.168 |
| 6 | EA00371 | CDPK9 | 1 | 100 | 97.42 | 96.168 |
| 7 | EA00990 | CDPK9 | 1 | 100 | 97.42 | 96.168 |
| 8 | Fla8924 | CDPK9 | 1 | 100 | 97.42 | 96.168 |
| 9 | Floradade | CDPK9 | 1 | 100 | 97.42 | 96.168 |
| 10 | LA2093 | CDPK9 | 1 | 99.241 | 99.94 | 99.266 |
| 11 | LYC1410 | CDPK9 | 1 | 100 | 97.42 | 96.168 |
| 12 | MM | CDPK9 | 1 | 100 | 97.42 | 96.168 |
| 13 | PAS014479 | CDPK9 | 1 | 99.187 | 99.88 | 99.815 |
| 14 | PI169588 | CDPK9 | 1 | 100 | 97.42 | 96.168 |
| 15 | PI303721 | CDPK9 | 1 | 99.982 | 97.42 | 96.168 |
| 16 | PP | CDPK9 | 1 | 99.187 | 97.29 | 96.869 |
| 17 | 1706 | CDPK9 | 1 | 100 | 100.00 | 100 |
| 18 | TS112 | CDPK9 | 1 | 100 | 97.42 | 96.168 |
| 19 | TS117 | CDPK9 | 1 | 99.638 | 93.48 | 95.292 |
| 20 | TS118 | CDPK9 | 1 | 100 | 97.42 | 96.168 |
| 21 | TS12 | CDPK9 | 1 | 100 | 97.42 | 97.232 |
| 22 | TS15 | CDPK9 | 1 | 100 | 97.42 | 96.168 |
| 23 | TS156 | CDPK9 | 1 | 100 | 97.42 | 96.168 |
| 24 | TS166 | CDPK9 | 1 | 100 | 97.42 | 96.168 |
| 25 | TS171 | CDPK9 | 1 | 100 | 97.42 | 96.168 |
| 26 | TS185 | CDPK9 | 1 | 100 | 97.42 | 97.053 |
| 27 | TS204 | CDPK9 | 1 | 100 | 97.42 | 96.168 |
| 28 | TS222 | CDPK9 | 1 | 100 | 97.42 | 96.168 |
| 29 | TS238 | CDPK9 | 1 | 100 | 97.42 | 96.168 |
| 30 | TS265 | CDPK9 | 1 | 98.734 | 93.85 | 93.358 |
| 31 | TS280 | CDPK9 | 1 | 100 | 97.42 | 96.168 |
| 32 | TS281 | CDPK9 | 1 | 100 | 97.42 | 96.168 |
| 33 | TS3 | CDPK9 | 1 | 100 | 97.42 | 96.168 |
| 34 | TS331 | CDPK9 | 1 | 100 | 97.42 | 96.168 |
| 35 | TS39 | CDPK9 | 1 | 99.187 | 97.29 | 97.048 |
| 36 | TS413 | CDPK9 | 1 | 99.187 | 97.29 | 96.869 |
| 37 | TS421 | CDPK9 | 1 | 98.734 | 90.59 | 90.037 |
| 38 | TS439 | CDPK9 | 1 | 100 | 97.42 | 96.168 |
| 39 | TS545 | CDPK9 | 1 | 99.187 | 96.06 | 95.764 |
| 40 | TS60 | CDPK9 | 1 | 100 | 97.42 | 96.168 |
| 41 | TS623 | CDPK9 | 1 | 99.187 | 97.36 | 96.869 |
| 42 | TS629 | CDPK9 | 1 | 100 | 97.42 | 96.168 |
| 43 | TS692 | CDPK9 | 1 | 100 | 97.42 | 96.168 |
| 44 | TS80 | CDPK9 | 1 | 98.734 | 96.92 | 95.438 |
| 45 | TS9 | CDPK9 | 1 | 100 | 97.42 | 96.168 |
| 46 | TS96 | CDPK9 | 1 | 100 | 100.00 | 100 |

Particularly, the CDPK8 genome identity among different varieties is 98.734-100%, the CDS identity is 92.83-100%, and the protein sequence identity is 93.007-100%; the CDPK9 genome identity among different varieties is 98.734-100%, the CDS identity is 90.59-100%, and the protein sequence identity is 90.037-100%.

Note: In this disclosure, CDPK8 gene is the abbreviation of Solyc11 g065660 gene, CDPK9 gene is the abbreviation of Solyc01 g008440.2.1 gene, and SuSy3 gene is the abbreviation of Solyc07 g042550.2.1 gene.

CDPK8, CDPK9, SuSy3, or CDPK8, CDPK9, SuSy3 indicating either a gene or a protein can be determined according to the context.

The reagents, instruments, strains or biological materials used in the present disclosure are all commercially available.

The present disclosure will be further described below in conjunction with Examples:

### Example 1: Knockout of CDPK8 Gene by CRISPR/Cas9 System

The protein sequence information of the tomato CDPK8 gene is as follows.

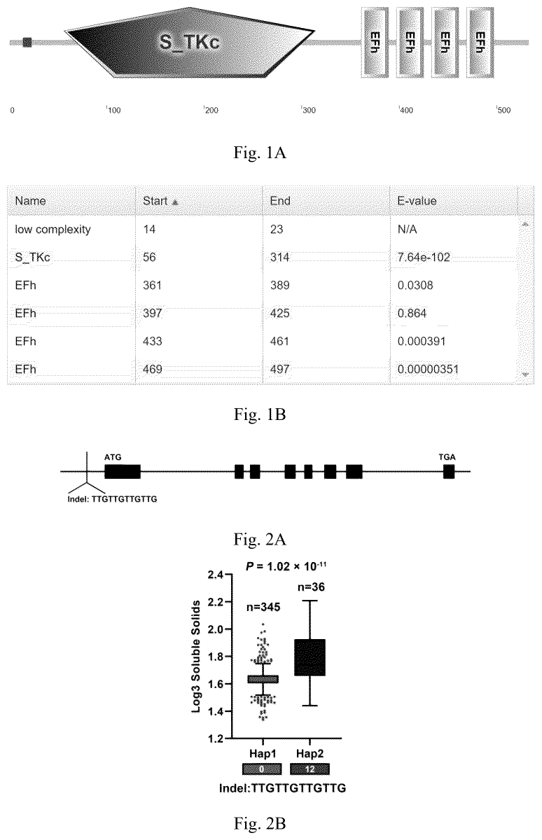

FIG. 1A shows prediction of the conserved domains of the tomato CDPK8 gene protein sequence. CDPK8 contains 533 amino acid residues. It includes a low-complexity region (LCR) at the N-terminus, a serine/threonine protein kinase domain (S_TKc) with protein kinase activity that can catalyze the phosphorylation of serine and threonine hydroxyl groups in proteins, and four EF-Hand domains at the C-terminus that can bind to calcium ions to cause changes in protein conformation. FIG. 1B shows detailed information on the location of conserved domains in the protein sequence of tomato CDPK8 protein. Data source: SMART website. In FIG. 1B, name indicates the name of the conserved domain, start indicates the starting position of the conserved domain, end indicates the ending position of the conserved domain, and E-value indicates the expected value

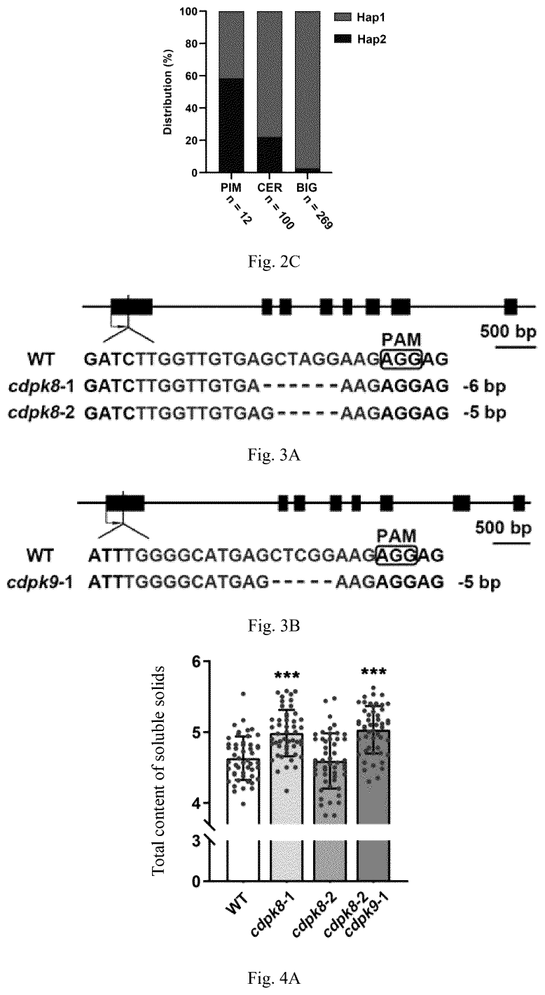

FIG. 2A shows the genome sequence information of tomato CDPK8. The full length of the CDPK8 genome is 4914 bp, including 8 exons and 7 introns. There is a 12 bp sequence variation in the upstream of its promoter (324-335 bp before the start codon ATG) that is closely related to the SSC. As shown in FIG. 2B, the 12 bp sequence variation can divide the natural tomato population into two haplotypes. Compared with the haplotype Hap1 without the 12 bp sequence variation, the SSC of the haplotype Hap2 with the 12 bp sequence variation is significantly increased, indicating that the presence of the 12 bp sequence can increase the SSC of tomato fruit. In addition, further analysis found that, as shown in FIG. 2C, the proportion of tomato materials with the Hap2 haplotype gradually decreased in Solanum pimpinellifolium (PIM), Lycopersicon esculentum var. cerasiforme A. Gray (CER) and BIG tomato (BIG) populations. In Solanum pimpinellifolium population, about 60% of the materials are of the Hap2 haplotype, while in the BIG tomato population, the proportion of the Hap2 haplotype is very low. Based on this, it is speculated that the deletion of the 12 bp sequence may be part of the reason for the reduction in SSC during tomato domestication and improvement.

The CRISPR/Cas9 system was used to knock out the CDPK8 gene in tomatoes and obtain a CDPK8 gene knockout mutant. The specific steps are as follows.

### Step 1: Selection of sgRNA Sequence

A target site sequence with a length of 19 bp was designed on the CDPK8 gene.

(CDPK8-CDS):
SEQ ID NO: 1
ATGGGGAATTGCTGTGGGACACCTGGTAATTCTTCTGAGAATAAGAAGAAGAAGAACAAACCA
AACCCTTTTGCTCTTGATTATGGTGCAACTCAAGCATCTGGAGGTGATGGAAACAAGCTTGTTG

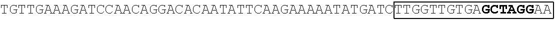

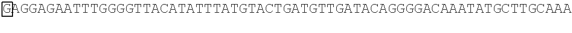

TCGATATCAAAGAAGAAACTTAGGACTGCTGTAGATATAGATGATGTTAGGAGAGAAGTTGAGA
TCATGAAGCATTTGCCTAAACATCCTAATATTGTGACCTTGAAGGACACTTATGAGGATGATAAT
GCAGTGCATATTGTGATGGAACTTTGTGAGGGGGGTGAGTTGTTTGATAGGATTGTTGCCAGGG
GACATTATACAGAGAGAGCAGCTGCGGTTATTATGAAGACTATAGTAGAAGTTGTTCAGATGTG
TCATATGCATGGAGTAATGCATCGTGATCTCAAACCTGAGAACTTTCTGTTTGGTAACAAGAAA
GAAACTGCTCCTTTGAAGGCTATTGACTTTGGATTATCAGTGTTCTTTAAACCCGGGGAACGCT
TTAATGAGATTGTGGGAAGTCCTTATTACATGGCTCCAGAGGTCCTAAAACGCAATTATGGCCCG
GAGGTTGATGTCTGGAGTGCTGGAGTTATCCTTTATATTCTTCTTTGTGGTGTTCCACCTTTCTGG
GCAGAGACTGAACAAGGAGTAGCCCAAGCAATTATTCGGTCTGTGGTTGATTTCAAGAGAGAT
CCATGGCCTAAGGTTTCTGATAATGCAAAGGATCTTGTAAAGAAGATGCTTGACCCGGATCCAA
CTCGACGACTCACAGCTCAGCAGGTTCTTGAGCACACCTGGTTACAAAATATAAAGAAAGCAC
CAAATGTCTCATTGGGTGAGACTGTGAAAGCAAGGCTTAAACAATTTTCAGTAATGAACAAGCT
CAAGAAAAGAGCTCTAACGATTATGGCCGAGTTTTTATCAGCGGAAGAAGTGGCTGGAATGAA
GGACGCATTTGATATGATGGATACAGGAAAGAAAGGCAAGATTAACCTTGGAGAACTTAAAAAT
GGTCTGCAAAAGCTTGGCCATCAGATCCCTGATGTTGATCTTCAGATTCTTATGGAAGCTGCGG
ATGTTGATGGAGATGGAAGCTTAAATTATGGGGAGTTTGTTGCTGTATCTGTTCATCTCAGAAAA
ATGGCAAATGATGAGCACCTGCACAAAGCATTTTCAGTTTTTGACAGAGATCAGAGTGGTTACA
TAGAAATCGAGGAGCTGCGCAGTGCCTTGAGTGATGAGGATGGTGGCAACAGTGAGGAAGTC
ATCAATGCCATTATGCATGATGTTGACACTGACAAGGATGGCCGCATTAGTTACGAGGAATTTGC
TGCAATGATGAAGGCTGGCACAGATTGGAGAAAAGCATCGAGACAGTATTCTCGTGAAAGATT
CAACAGTCTCAGCTTAAAATTGATGAGAGATGGCTCGATACAAGTCGGAAAGGAAGAAGGTAG
ATGA.
(CDPK8-protein):
SEQ ID NO: 2
MGNCCGTPGNSSENKKKKNKPNPFALDYGATQASGGDGNKLVVLKDPTGHNIQEKYDLGCELGR
GEFGVTYLCTDVDTGDKYACKSISKKKLRTAVDIDDVRREVEIMKHLPKHPNIVTLKDTYEDDNAV
HIVMELCEGGELFDRIVARGHYTERAAAVIMKTIVEVVQMCHMHGVMHRDLKPENFLFGNKKET
APLKAIDFGLSVFFKPGERFNEIVGSPYYMAPEVLKRNYGPEVDVWSAGVILYILLCGVPPFWAETE
QGVAQAIIRSVVDFKRDPWPKVSDNAKDLVKKMLDPDPTRRLTAQQVLEHTWLQNIKKAPNVSLG
ETVKARLKQFSVMNKLKKRALTIMAEFLSAEEVAGMKDAFDMMDTGKKGKINLGELKNGLQKL
GHQIPDVDLQILMEAADVDGDGSLNYGEFVAVSVHLRKMANDEHLHKAFSVFDRDQSGYIEIEEL
RSALSDEDGGNSEEVINAIMHDVDTDKDGRISYEEFAAMMKAGTDWRKASRQYSRERFNSLSLKL
MRDGSIQVGKEEGR.
(CDPK8-genome):
SEQ ID NO: 3
ATGGGGAATTGCTGTGGGACACCTGGTAATTCTTCTGAGAATAAGAAGAAGAAGAACAAACCA
AACCCTTTTGCTCTTGATTATGGTGCAACTCAAGCATCTGGAGGTGATGGAAACAAGCTTGTTG

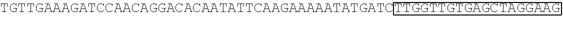

AGGAGAATTTGGGGTTACATATTTATGTACTGATGTTGATACAGGGGACAAATATGCTTGCAAAT
CGATATCAAAGAAGAAACTTAGGACTGCTGTAGATATAGATGATGTTAGGAGAGAAGTTGAGAT
CATGAAGCATTTGCCTAAACATCCTAATATTGTGACCTTGAAGGACACTTATGAGGATGATAATG
CAGTGCATATTGTGATGGAACTTTGTGAGGGGGGTGAGTTGTTTGATAGGATTGTTGCCAGGGG
ACATTATACAGAGAGAGCAGCTGCGGTTATTATGAAGACTATAGTAGAAGTTGTTCAGGTACAG
TTTTACTAATTAATGCTTGTCTTTGCTTTTCATCTATAATTGTTACTTGTTTGTTTATTTCATGTAAT
TTAGGGGTTGCTAATTTAGGATTATGTTAATGAAGTTTGAATTGACAATGCCAACTGAAAGTGTC
AATTTAGACTAGAAAGGAATGTTGAGATTGAAGTTTCATGTTTGTAAGCAGAATGAATGTTCTT
GTCGAAGAATCTCTTTTAAGAATATCTTTCCCACTTCTTCAGTAAATTTTCCTACTTTCTTATGTC
GATAACTGCCACTCCTTTTCTGAGTTTGCCTGCACTCTCTTGGTAGATAGGACACTCCCTTTCTT
AGCTCAGGATCTACTTTGCTTGTGTAAATGAGTGCTTAATGTTTTTATTTGATTCATTTTACACGT
TGAGATCCTATAGCATTTAATTAGACTAGATTTTATCTTCCAATATCACTAAACCTAGATGTCAAA
TATAGTAAAAAGTTATGTTGCTCGAACTCTTCAAAGATATCGACATGTGTGTGTCAGATCCTCCA
AAAGTAGTGCATTTTTGGAGGATCCGACGGGGGTGGGGCAACAATTTTGGAGAGTTCGAGCAA
CTTAGGTAAAAAGTGTATTCATGCTCTAAATGTTGTTAGGTTTTCTATCTTTCTTATCTTGTCTTGT
GAATTTTGCTTTCTTGGCAACTGTTTCATTGTCCTATATTTTTCTCATGCAAATACCTTTAAAATTA
TATACTTTCGTGCCAACCATTTAAATGAAACTTAAGGAAGCTAATCACACAGGATTTCACCATCC
AAATGTCGAAAATGAATCGCAATTTGTGATAGTTTTAGACAGAGAGCTTATTTTTATAGATGCAA
TTAGAATAATAGAAATGTAAAATAACTATCAGTAACCAGTAAGATGCCGGTCTTTATGGTCTATTT
TCAGAGGGGGAGATCTATTGTAGCTTGCTTTCAGCATCAGTTAGTATAGCATAGGACTGTTGCTA
ACAGGGTTCAATTTTAAACTGTATTTGGTGAACATAATGACATGAGAGCTCAACTTACATAACTA
CAGATTAGAGAGAGACATTCATTTTATGGAATGGATTTAAGAAGTTCATGTTTCTACATTTAGTT
GTTATAAGAACAGGGTGGTAACTGGTAACCGACGCAAACATGATGGCTAATTATCCCAAGTTTA
TACAATTTAATCTATGTGGTTGCTCATACATCTTTGGTCAATGTTCACTTTACAGTTGCATTATGTT
TCCTCAAGAAATGGCTGTTCTTCTTTCAATTTTCAACTTACGGAGTTTCAATTGACATTGCAGAT
GTGTCATATGCATGGAGTAATGCATCGTGATCTCAAACCTGAGAACTTTCTGTTTGGTAACAAG
AAAGAAACTGCTCCTTTGAAGGCTATTGACTTTGGATTATCAGTGTTCTTTAAACCCGGTAATGC
AATAAGTAATGATAGTGTGTTGTAACTTTTTTTCATGCTAAAAAAGATTAATGTTGGAATCATCTT
TTAATATGTTTAACAGGGGAACGCTTTAATGAGATTGTGGGAAGTCCTTATTACATGGCTCCAGA
GGTCCTAAAACGCAATTATGGCCCGGAGGTTGATGTCTGGAGTGCTGGAGTTATCCTTTATATTC
TTCTTTGTGGTGTTCCACCTTTCTGGGCAGGTCCGTTTCTTTTATTTGCCTTCTTTAGGTAACAA
GTAATACCTCGGTTCACCGACAAATAACAAAGTAAATCAACCTTGGTGCATACTATTCATTTCTT
AGAACTTGGTAATTTATTAATGAGTGTCAAAATGAAATGGAATTTGAAACATATTGATGCGTTGT
TAACATCGTCGATGACTAGGTGAGGGTGGCTGCAAGAGTGAATCCCACCTCTTCCTGGACCAAT
TCAAAATCAAAGTTCCTTTTTTTCCTTTTGGCCTGTGATAGTCAGTACTTTGTGCTTCCATTAATT
TACTCACAGCTTTCTCAGTAATTGCCTAAAATTCTACTGAATATGTGCAGAGACTGAACAAGGA
GTAGCCCAAGCAATTATTCGGTCTGTGGTTGATTTCAAGAGAGATCCATGGCCTAAGGTTTCTG
ATAATGCAAAGGATCTTGTAAAGAAGATGCTTGACCCGGATCCAACTCGACGACTCACAGCTCA
GCAGGTTCTTGGTAATGTTTGTTAATCCTTGAGATGCTATACTTTCATTTGCTTGCTGTTTGTTCT
CTCTTAGTTTCATAAGTTCAATTTTGTGACTTTTTTGGGGCTTTCGTGCATATACATGAATGCAGA
GCACACCTGGTTACAAAATATAAAGAAAGCACCAAATGTCTCATTGGGTGAGACTGTGAAAGC
AAGGCTTAAACAATTTTCAGTAATGAACAAGCTCAAGAAAAGAGCTCTAACGGTAAAATATTCC
CTTTTCATTTGCAGTTACATATCATTAGCGTTCTGCACCTTCTCAGAAGGATATCTTCATATGCCT
AATTCTTAAAGCCCAAAATAAGGACAAAAGATTTTCTGCCTTCTCACAAAAGTTTCGGGTGTTA
TATTCTTTCTTCATTGCTTGCACAGATTATGGCCGAGTTTTTATCAGCGGAAGAAGTGGCTGGAA
TGAAGGACGCATTTGATATGATGGATACAGGAAAGAAAGGCAAGATTAACCTTGGAGAACTTA
AAAATGGTCTGCAAAAGCTTGGCCATCAGATCCCTGATGTTGATCTTCAGATTCTTATGGAAGCT
GTAAGCATTCTCATGCTCTTCATTTCACTTGATTGGATTTTCTTTCAACTTTACGAAGACTAAATA
TATATATTTATAAGTCAAGCGTCTAATATTATTATAATTGTTCCCTTTTTTATCTTCTGCTTCACTTT
TATAGGCGGATGTTGATGGAGATGGAAGCTTAAATTATGGGGAGTTTGTTGCTGTATCTGTTCAT
CTCAGAAAAATGGCAAATGATGAGCACCTGCACAAAGCATTTTCAGTTTTTGACAGAGATCAG
AGTGGTTACATAGAAATCGAGGAGCTGCGCAGTGCCTTGAGTGATGAGGATGGTGGCAACAGT
GAGGAAGTCATCAATGCCATTATGCATGATGTTGACACTGACAAGGTTAGAATTTTTTTTGTGTC
CATTCATGTATATCTACTGGATTTCATACTAGTGCATATACTTCTTTTATAAATGTCTCTTTAATTGC
TTGTGGAACCATAAATACCCCCCTTATCTTGTTCCGCAGCTCCAACTACACACGAAACTATGTTG
AAGACGTATTACCCCCTTGTACTTGTAAAATGTGTATCTTCCGTCATCCTGAGAGGCATATATGAT
GTAACCAAATGTGAACATTTAAGTATATGCATGTGCTGCCTCATGTCATTTACCCCTTTTTCCCTT
TTCACTTCTCTTCCATCTCCGCAACATGTTAATGTCCTCTAGCAAATCTGAAAAAAGATGGAGTG
AGCTTCTCAACGGAAAAAATCGGATTCACCCTAGTTTTAGTTTTCTGGCAACATAAACAGCTCA
AGTCGATACAAAAAAACAAATGTTCCTATCAAAAAACTCAAATTAGTAGAAAGGAAAAGAAGG
GTCATAGGTAAGGGAAGAACAAAAAGGAATAGAAAGACCCTTCGGGGTGGCCCAGTTATTTGG
GCTTGGGGCTTGGGGCTTGGGACTTCCATGTTGGAGGTCTCAAGTTCAAAACCCCTTGCCAATG
AAAGCAACGGGTTTTCCTTCTACGTTGACTGTCGAGCTCGTCGCACCGGGCTTACCTAGTGCGG
TTTACCTCTCCTTTGCGTGCTATAGGAATAGGGGTTTTACCCTGTGTGCACTCAAAGGGTAGCGA
CTGCGGATTTCCCAAAAGAAAAAGGAAGAGAAAGGTAAAGAAAACATTTTAAAAAAGAGTAG
GATGGTTATAATAGAAGAAATGCTACAATATTTTTTCACCTCAGGCGCTTTAAGAGAGTAAATAC
TCTACGTTACATGGCGTCCCAATGTTGTCGTAGTTACATTTTGGACAAGAACAGGGAGTAATAG
GCCTCCCGCAAAGTTAAAATGTAGTGGGGAATGGGGGAATAATTGAGGAAGGTATTTATATATTT
TCTCCTTTCTAAATCATATTTTCTTTTCCATTCACTGCTCTTCTGTTCAACCTTATCATTCATTTGG
ACTAAATGTTGAATTGATTTTGAGCTTTTAGTTTGTTACCACAGCTTCAAAGTTCATCAGTTACA
TTGTCTATAACCATGCAGGATGGCCGCATTAGTTACGAGGAATTTGCTGCAATGATGAAGGCTG
GCACAGATTGGAGAAAAGCATCGAGACAGTATTCTCGTGAAAGATTCAACAGTCTCAGCTTAA
AATTGATGAGAGATGGCTCGATACAAGTCGGAAAGGAAGAAGGTAGATGA.

The target site is located at positions 173-191 of SEQ ID NO: 1 (CDS sequence) and at positions 173-191 of SEQ ID NO: 3 (genomic sequence). The sequence of target site 1 is TTGGTTGTGAGCTAGGAAG (SEQ ID NO: 4 corresponding to the sequence shown in the box in SEQ ID NO: 1 and SEQ ID NO: 3).

The sgRNA sequence designed for the target site is:

(SEQ ID NO: 5)
GUUUUAGAGCUAGAAAUAGCAAGUUAAAAUAAGGCUAGU
CCGUUAUCAACUUGAAAAAGUGGCACCGAGUCGGUGC.

The encoding DNA molecule of this sgRNA is:

(SEQ ID NO: 6)
GTTTTAGAGCTAGAAATAGCAAGTTAAAATAAGGCTAGT
CCGTTATCAACTTGAAAAAGTGGCACCGAGTCGGTGC.

### Step 2: Construction of CRISPR/Cas9 Vector

The original vector contains the sgRNA sequence, and the target site 1 sequence in step 1 is further inserted into the vector to obtain a CRISPR/Cas9 vector.

### Step 3: Obtaining Transgenic Plants

The CRISPR/Cas9 vector obtained in step 2 was transferred to Agrobacterium competent cells EHA105 by heat shock transformation (Agrobacterium EHA105 competent cells were purchased from Shanghai Weidi Biotechnology Co., Ltd. and are available to the public) to obtain recombinant bacteria EHA105/CRISPR/Cas9.

The recombinant bacteria EHA105/CRISPR/Cas9 were then used to transform tomatoes by transformation method of Agrobacterium-infection (the recombinant Agrobacterium was propagated at 28° C., and the propagated bacterial solution was used to infect tomatoes). After kanamycin resistance screening, the T₀ generation of transgenic tomato plants were obtained.

### Step 4: Identification of Transgenic Plants with Mutations in the CDPK8 Gene

The leaves of the T₀ generation transgenic tomato plants obtained in step 3 of Example 1 were collected, and genomic DNA was extracted as a template. PCR amplification was performed by using the following primer pairs to obtain PCR amplification products of different strains.

The sequences of the primers for detection of a CDPK8 mutation sequence are as follows.

CDPK8-test-F:
(SEQ ID NO: 7)
ATGGGGAATTGCTGTGGGAC.
CDPK8-test-R:
(SEQ ID NO: 8)
TTCATAATAACCGCAGCTGCTCTCTCT.

The PCR amplification products of different strains were subjected to Sanger sequencing, and the sequencing results were compared with the wild-type CDPK8 gene. The CDPK8 genotypes were identified according to the following principles.

If there is a sequence with a double peak characteristic from the target site sequence, the genotype of the strain is a heterozygous genotype (the CDPK8 gene on one of the two homologous chromosomes is mutated, and the CDPK8 gene on the other chromosome is not mutated), and the strain is a TO generation transgenic tomato heterozygous mutant strain.

If there is a double peak sequence starting from the target site sequence and the CDPK8 gene in both homologous chromosomes are mutated, then the strain is a T₀ generation transgenic tomato biallelic mutation strain.

If the sequence with a specific single-peak characteristic starting from the target site sequence is the same as the CDPK8 gene sequence of the wild-type tomato, the genotype of the strain is wild type, that is, the CDPK8 gene sequence has not mutated; if it is different from the sequence of the CDPK8 gene of the wild-type tomato, the genotype of the strain is a homozygous genotype (both the CDPK8 genes on the two homologous chromosomes are mutated), and the strain is a T₀ generation transgenic tomato homozygous mutant strain.

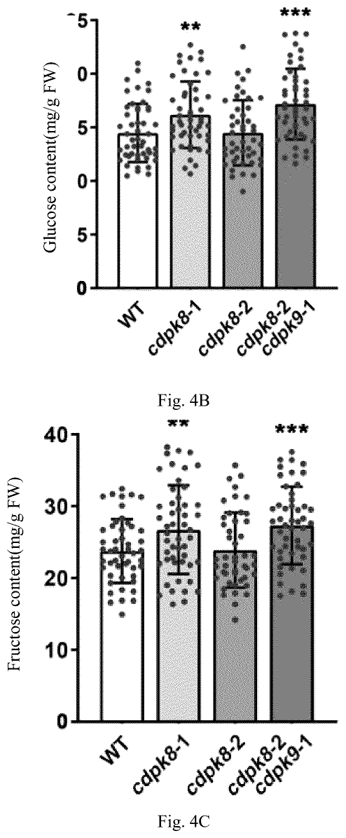

In this case, a homozygous mutant strain of the CDPK8 gene of the TO generation was identified (as shown in FIG. 3A and FIG. 3B), which was used to identify the tomato SSC and sugar content phenotypes described below.

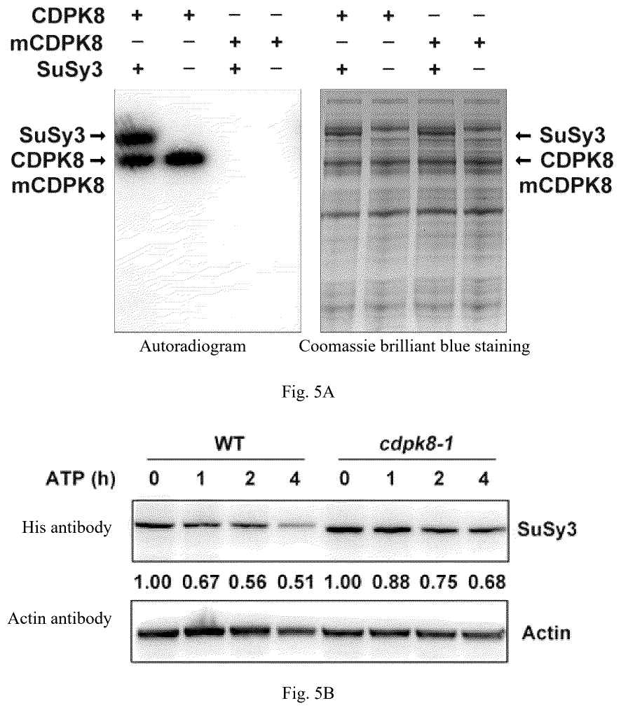

FIG. 3A shows cdpk8-1 genotype 1 and cdpk8-2 genotype of the homozygous mutant strains of tomato CDPK8 gene; compared with the wild type (WT), the cdpk8-1 mutant strain has a 6-base deletion mutation, and the missing 2 amino acids are necessary for the CDPK8 protein to maintain kinase activity. After the deletion, it loses its kinase activity (as shown in FIG. 5A, mCDPK8), and the cdpk8-2 mutant strain has a 5-base deletion mutation, and the CDPK8 protein translation will undergo a frame-shift mutation, so cdpk8-1 and cdpk8-2 are both loss-of-function mutants of CDPK8 gene.

### Example 2: Knockout of the CDPK9 Gene by CRISPR/Cas9 System

CRISPR/Cas9 system was used to knock out the CDPK9 gene in tomatoes and obtain a CDPK9 gene knockout mutant. The specific steps are as follows.

### Step 1: Selection of sgRNA Sequence

A target site sequence with a length of 19 bp was designed on the CDPK9 gene.

(CDPK9-CDS):
SEQ ID NO: 9
ATGGGTAATTGCTGTGTGAAACCGGGTAAATCTGCCGAAAAAAAGAATAAAAAGAACAACAGC
AAACCTAACCCTTTTTCAATTGATTATGGGGGGACTAAACATGCCTCTGGGAGTGGAAACAAGC

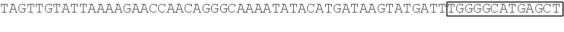

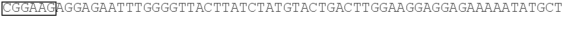

TGCAAATCGATATCTAAAAAGAAGCTAAGGACTGCAGTGGATATTGATGATGTTAGGAGAGAGG
TTGAGATCATGAAACATTTGCCTGTGCATCCGAATATTGTGACCTTGAAGGATACTTATGAGGAT
GATAATGCAGTGCACATTGTGATGGAATTGTGTGAGGGTGGGGAGTTGTTTGATCGAATCGTTG
CTAGAGGCCACTATACAGAGAGAGCAGCTGCTGGAATTTTGAAGACTGTCGTGGAAGTCGTTC
AGATGTGTCACAGGCAAGGGGTAATGCATCGTGATCTCAAACCTGAGAATTTTCTTTTTGGTAA
CAAGAAAGAAACAGCTCCACTGAAGGCTATTGACTTTGGGTTGTCTGTTTTCTTTAAACCTGGG
GAACGCTTTAATGAGATAGTGGGAAGTCCTTATTACATGGCTCCTGAGGTCCTAAAGCGCAATT
ATGGACCAGAGGTCGATGTCTGGAGTGCTGGAGTTATACTTTACATTCTTCTATGCGGTGTTCCA
CCTTTCTGGGCAGAGACTGAACAAGGAGTAGCCCAAGCGATTATTCGTTCTGTGATTGATTTCA
AGAGGGATCCATGGCCTAAAGTTTCTGATAATGCAAAGGATCTTGTAAAGAAAATGCTTGATCC
AGATCCAACTCGACGGCTCACAGCTCATCAAGTTCTTGAGCATCCCTGGTTACATAATATAAAG
AAAGCACCAAACGTCTCATTGGGTGAGACTGTTAAAGCAAGACTCAAGCAGTTTTCAGTAATG
AACAAGCTCAAGAAAAAAGCTCTGACGGTTATAGCTGAGTTTTTGTCTGCGGAGGAAGTCGCT
GGAATGAAGGAAGCATTTGAAATGATGGATACCGGAAAGAAGGGCAAGATAAACCTGAATGAA
CTTAAAGATGGCTTGCAGAAGCTTGGCCATCAAATCCCTGATGCTGATCTTCATATTCTCATGGA
AGCGGCTGACGTTGATGGAGATGGAAGTTTAAATTATCCAGAGTTTGTTGCTGTATCTATTCATC
TTAGAAAGATGGCCAATGATGAACACCTGCACAAAGCATTTTCATTTTTCGACAAAAATCAGAG
TGGTTTCATAGAAATCGAAGAGCTTCGTAGTGCTTTGAGGGATGAAGACGACAGCAACAGCGA
GGAAGTCACCAATGCCATTATGCATGACGTTGATACAGACAAGGATGGTCGGATTAGTTATGAG
GAATTTGCTGCGATGATGAAGGCTGGTACGGACTGGAGAAAAGCATCGAGACAGTATTCTCGT
GAACGTTTTAACAGTCTAAGCTTAAAGTTGATGAGGGAAGGCTCATTACAAGTTGAAAACAAA
GTCTAG.
(CDPK9-protein)
SEQ ID NO: 10
MGNCCVKPGKSAEKKNKKNNSKPNPFSIDYGGTKHASGSGNKLVVLKEPTGQNIHDKYDLGHEL
GRGEFGVTYLCTDLEGGEKYACKSISKKKLRTAVDIDDVRREVEIMKHLPVHPNIVTLKDTYEDDN
AVHIVMELCEGGELFDRIVARGHYTERAAAGILKTVVEVVQMCHRQGVMHRDLKPENFLFGNKK
ETAPLKAIDFGLSVFFKPGERFNEIVGSPYYMAPEVLKRNYGPEVDVWSAGVILYILLCGVPPFWAE
TEQGVAQAIIRRSVIDFKRDPWPKVSDNAKDLVKKMLDPDPTRRLTAHQVLEHPWLHNIKKAPNVS
LGETVKARLKQFSVMNKLKKKALTVIAEFLSAEEVAGMKEAFEMMDTGKKGKINLNELKDGLQK
LGHQIPDADLHILMEAADVDGDGSLNYPEFVAVSSIHLRKMANDEHLHKAFSFFDKNQSGFIEIEELR
SALRDEDDSNEEVTNAIMHDVDTDKDGRISYEEFAAMMKAGTDWRKASRQYSRERFNSLSLKL
MREGSLQVENKV.
(CDPK9-genome):
SEQ ID NO: 11
ATGGGTAATTGCTGTGTGAAACCGGGTAAATCTGCCGAAAAAAAGAATAAAAAGAACAACAGC
AAACCTAACCCTTTTTCAATTGATTATGGGGGGACTAAACATGCCTCTGGGAGTGGAAACAAGC

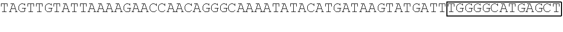

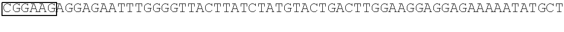

TGCAAATCGATATCTAAAAAGAAGCTAAGGACTGCAGTGGATATTGATGATGTTAGGAGAGAGG
TTGAGATCATGAAACATTTGCCTGTGCATCCGAATATTGTGACCTTGAAGGATACTTATGAGGAT
GATAATGCAGTGCACATTGTGATGGAATTGTGTGAGGGTGGGGAGTTGTTTGATCGAATCGTTG
CTAGAGGCCACTATACAGAGAGAGCAGCTGCTGGAATTTTGAAGACTGTCGTGGAAGTCGTTC
AGGTATAGTTTTTATCAACTTCTTAGTTCTCAGTTACCTACTGCTTTTATTACCTTTTGCTTAGGTT
ATCTTATTATATCCATTATTTTCAGCATAGCTTCTTCATTACTGTATTTCCTTTTCATATTGTTTTTAT
ATATGTCTTACTTAGGCTGAGGGTCCATTGGAAATAGCCTCTCTACCTTCACAAGGTAGGAGTAA
GGCTGCGTACACACTACCCTTCCCACACCCCACTTGTGGGACTATACTAAGTATGTTGTTGTTTT
AGTTCTCAATTGATGCTTTTTGGTTTTTATGTAGTAACTTGTGGTGTTTCTTTTACTGAGTTCTTC
AAAGCTTTTGTATGGGTTTTATATCATGACGGATGTATCCTGTATTGTTGTTTCGGGTTTTTATTAA
AGAAGTCTGGCAATTTCAAGTTAAAAAGAAAATCATATCTTAAAAGAGAAGCCTAATCTTGAGC
AAATCTTGAGCAGATAGCAATTTCAGGGTGAAATGTGAATATTCTTGGTGAAGTTGCATATGAA
CTGGTAACCATTATTCAATTCATTTCTAGTGTTGATCTTTGCAATCAATGGTTTCATTCTAGTGTA
CGTTCATGCTGTAGCATTTACCTTGATATCCTCATGGAATTTCCTTTACTGCCAAAATTATAATGA
GAAGTGGTTATCTTTTTTAATAATGGTAAAGTATGAGAAGAGGTTTTCTTATGTTCTCAGTCTTTG
CTAGTTCTATTTCTTTACTCGTTTTACTTAACTTTTTTAGTTTCTTGTGAACTGTAGTATTGCTCTA
TATCTTCTCACTCAAATTCATGTTCTAAATCTTAACCATTCTAGTGATATAAATGAAGTTAATTGG
TGATATAGGATATCCCCCTCCAAAATCTAACAATCTTTCAACTCATCATAGTCTTGGGCAGATAGC
TAGTTAGAACCGTAACAAATATTTAGGAAAGTAGATCATTCGAAATAGTTATCCTTGTGAAATAT
TATTCTTTATCTTCATTATTGACCATTGGTTTTCAAACTCTAGTACTCTTAAGTGGAAGACTCACT
TCTTGTTTCTGTGGAGTATTAGCTATAGCGGCCTACAACAGAATTTGATTGTTGCACTCAAAGTT
AAACAGAGTTCTACAGTTTAGGGAAATGATAATGGAATTTAAATTTAGTTTGTATACTGAAGTAA
ATATGGTTGACACTATCAGAAACAAGTGCTTTTTACTTCAAATATTGATCTTTATCCTCATTGCGA
ACCATTGATGTCCAAACTGTTTTAGATGGAAGAGCAATTTGTTGTTCATTTGAGTGGAATAGTCA
CCAACAACAGCTAATTAACTACGTATAGCTTAGAGAGATGCGAACCAATGGTAAATTTAAGTTG
TCTCCTGAAGTAGTTACTGATTGTTCATGATAAAGGTCATTAAAGTTGAATTCTCTATTTAGTCAG
TGTCCTTTTCTGCATCGACTATATAGTGGCCTGAATGACATTACAAAATATTTATGAGTTGGTAGG
AATCTTTTTTTCATATGTGAATCATGAATGTGTTCACAAATGATCAATCAGAGAGTGATTATTGTA
TTATGTGGTGTTGATAACTTTTGAACTTTTCTTCATTTGGTTTGTGTTTAACTGTTATCTGAAAGG
CTTGGTTGGTGGTACTAAAGCTATTGGATTAGCAGAAAACTTTATACATGTTTATGGTCATGTTTC
GCTCTCCAGTCCCCGTTTTTATTTGTCAAAGGTCAGTTCATATATTTCCAATGGTTTTGCAGATGT
GTCACAGGCAAGGGGTAATGCATCGTGATCTCAAACCTGAGAATTTTCTTTTTGGTAACAAGAA
AGAAACAGCTCCACTGAAGGCTATTGACTTTGGGTTGTCTGTTTTCTTTAAACCTGGTAATGGA
ATTGGAAAAAAAACAGTGAGTTTAAGACTTTTATCATGCTAAACAAAGGTTTAACATGTTGATT
ATTTTAACAGGGGAACGCTTTAATGAGATAGTGGGAAGTCCTTATTACATGGCTCCTGAGGTCCT
AAAGCGCAATTATGGACCAGAGGTCGATGTCTGGAGTGCTGGAGTTATACTTTACATTCTTCTAT
GCGGTGTTCCACCTTTCTGGGCAGGTCTGCTTATTTTATTTCTTCGTTTTTTATAACCATGGAACG
AACCTCAATGCAGATATTTTCATGTGCAATTTTTGGAACTTGATAATTGATTAACTAAGATTGTGA
ATATGAAGTAGAACTTGATTCATGTTAGAATATGTTGTTGAAATATTAGATGATGAGGCAAGTGA
GGTGAAAGACTGTCCCTGCTTTAATCAATGTAGATCATGTTCAATTATTTTGGTTTCTCTTAATGG
TTCTTATGGGTTGGACTTTGCTAAATAACTTGCATAAATATGTTAACATATTCTTGATACTTCCCTT
AAGTTCCTCTGAATATTTGCAGAGACTGAACAAGGAGTAGCCCAAGCGATTATTCGTTCTGTGA
TTGATTTCAAGAGGGATCCATGGCCTAAAGTTTCTGATAATGCAAAGGATCTTGTAAAGAAAAT
GCTTGATCCAGATCCAACTCGACGGCTCACAGCTCATCAAGTTCTTGGTAATGTTTGTGGAATAT
TGAGATATTGTACTTTATTCATTCGTTGTTTTACTCTCTCTCTCTCTCTCTCTCTCTCTCTATCTATC
TCTGATAAGTATTTTTTTCCGGGGCTTTGACTCTTGTGGTGCAGAGCATCCCTGGTTACATAATAT
AAAGAAAGCACCAAACGTCTCATTGGGTGAGACTGTTAAAGCAAGACTCAAGCAGTTTTCAGT
AATGAACAAGCTCAAGAAAAAAGCTCTGACGGTAAGCACACACTTAGTCTGCAATCATATTTAT
AAGCTCTGGATTCTTCACTGTCCACTAAAGATGAATTCATATGCTTTTCCTCATAGATCGAAATG
AAGGAGAGAAGGTTGTGTCTTAATTATTTACTGCACTGCCTTGTGCTATTTAATAAAAACTTAAT
TCTCATCGTAAAAAATGAGAGGCGGTTCTCAAAATTTTGATCAAGTTTCGTTTTTTTATCTTGAT
ATATGTACTTTCTTGTATTCCAAACAGGTTATAGCTGAGTTTTTGTCTGCGGAGGAAGTCGCTGG
AATGAAGGAAGCATTTGAAATGATGGATACCGGAAAGAAGGGCAAGATAAACCTGAATGAACT
TAAAGATGGCTTGCAGAAGCTTGGCCATCAAATCCCTGATGCTGATCTTCATATTCTCATGGAAG
CGGTAAGCGGTCTCATGCCCTGCTTCTTGGCTCGATCAGATTTCTTATAGCTCTACCGGGGGTAA
CATAATATTTGAAAGTTAGCTGCTAAACTTTTCTGCCTCAAAGAGAAAACAGGTGCCTGAGATG
ATATGGCCATGTCTTACGTAGAACTGTAAATTACATCAATGGACATTATTTTAAAATGACCGAAA
ACTACATGGAATCCATGTGAACTTAGTAAAGAAAATATTCAATGGAAGTAAAGGATACCAAGTA
GTTGGATTAAGGCTAGATTGTTGGTGGTACACTGACATTAGTGTCTCAATGTTTACTAAGAGTCT
CAGTTTGATTAGAGATTTGCACGTAGATGCAGGGATAACCGTGAGATTAAGAAAATTTTAAGAA
ATTCACGGAGATGAAAAAGATCTGTAATACTCATATAGCTTAGGGCCAAGCCCAACTAGTTTGG
GATCAAATCGTAGTTGTTGTTGATAAGAATTTAGCATTAGACTTTTGTCTGTCACCCTACTGTCA
AAAAATTGATTGATTACAAAATATATTTTGTCAATATAAGTTGTAACATTTTTCTACCATTTCAATG
TAGAAAAAAGGAAAATCTGTAGCAAAATTAGCTTGACATTTCGCTTGTCTTAGCATCCCTGGTT
CAATGCTTATTATTATCCAGAAAGGAAAAAAAAAACATTTCTCTTGATCCTTTTGCATCTTCGAC
CTGAAGGATGCTTATCTTTTTTATTATTTTATTTCCTTGAACTTTTGTTTTATGTTTTTCTTATTCTG
TATCGGTTTTACAGGCTGACGTTGATGGAGATGGAAGTTTAAATTATCCAGAGTTTGTTGCTGTA
TCTATTCATCTTAGAAAGATGGCCAATGATGAACACCTGCACAAAGCATTTTCATTTTTCGACAA
AAATCAGAGTGGTTTCATAGAAATCGAAGAGCTTCGTAGTGCTTTGAGGGATGAAGACGACAG
CAACAGCGAGGAAGTCACCAATGCCATTATGCATGACGTTGATACAGACAAGGTCAGTATGTCC
ATTTATGTGCACGTACACCATTTAGTAACTTTTCGTTCTTTGGTTCATACTTCATAGTGCAAATATT
ATGCATTGTTGCTAATGTTTACATCTTAATGAGTTCTCATTTTGAAGTTCTCTTCTGAACCCTCGT
TTGAGTTTAGAGCTCAAATACTCCACCTAGTTGTGACTTATTGTATCTCTAAAGTCCGTCATATTA
TATAAACGTATTATACTTTGATGGCATGCACACTTTCTAGCACATATGTGTTGCTAACGAAACTCT
AGTGCTGTGACAAATGAAATACATGTATACATTGATGGCATCTGTTCTTCCCATCACATATGTACT
ACCGGTCATTACTCTCTAGTGCTGTGACATTATGCTCTCCCTGTTCGCTTCATGAGTTACACGGT
TGGTTAACCTGCTGCTCGTTACATGTCTGCTACAGAATTCAAGTTTTGTTAGCTGTTTTCCATCTC
ATCTCTAACAACATCCCCACTCATTTGTATTAGCTTCTTATCTGGAGTTTTGTGAGCTCAGTCTTG
TATCGTCTTGTGTCAAAACGTGTGCAGGATGGTCGGATTAGTTATGAGGAATTTGCTGCGATGAT
GAAGGCTGGTACGGACTGGAGAAAAGCATCGAGACAGTATTCTCGTGAACGTTTTAACAGTCT
AAGCTTAAAGTTGATGAGGGAAGGCTCATTACAAGTTGAAAACAAAGTCTAG.

The target site is located at positions 179-197 of SEQ ID NO: 9 (CDS sequence) and at positions 179-197 of SEQ ID NO: 11 (genomic sequence), and the target site 2 sequence is TGGGGCATGAGCTCGGAAG (SEQ ID NO: 12, for example, the in-frame sequence in SEQ ID NO: 9 and SEQ ID NO: 11).

The sgRNA sequence designed for the target site is:

(SEQ ID NO: 13)
GUUUUAGAGCUAGAAAUAGCAAGUUAAAAUAAGGCUAGUC
CGUUAUCAACUUGAAAAAGUGGCACCGAGUCGGUGC.

The encoding DNA molecule of this sgRNA is:

(SEQ ID NO: 14)
GTTTTAGAGCTAGAAATAGCAAGTTAAAATAAGGCTAGTC
CGTTATCAACTTGAAAAAGTGGCACCGAGTCGGTGC.

### Step 2: Construction of CRISPR/Cas9 Vector

The original vector contains the sgRNA sequence, and the target site 2 sequence in step 1 was further inserted into the vector to obtain a CRISPR/Cas9 vector.

### Step 3: Obtaining Transgenic Plants

The CRISPR/Cas9 vector obtained in step 2 was transferred to Agrobacterium competent cells EHA105 by heat shock transformation (Agrobacterium EHA105 competent cells were purchased from Shanghai Weidi Biotechnology Co., Ltd., and are available to the public) to obtain recombinant bacteria EHA105/CRISPR/Cas9.

The recombinant bacteria EHA105/CRISPR/Cas9 were then used to transform tomatoes by transformation method of Agrobacterium-infection (the recombinant Agrobacterium was propagated at 28° C., and the propagated bacterial solution was used to infect tomatoes). After kanamycin resistance screening, the T₀ generation transgenic tomato plants were obtained.

### Step 4: Identification of Transgenic Plants with Mutations in the CDPK9 Gene

The leaves of the T₀ generation transgenic tomato plants obtained in step 3 of Example 2 were collected, and genomic DNA was extracted as a template. PCR amplification was performed by using the following primer pairs to obtain PCR amplification products of different strains.

The sequences of primers for detection of the CDPK9 mutation sequence are as follows.

CDPK9-test-F:
(SEQ ID NO: 15)
AGAACAACAGCAAACCTAACCC.
CDPK9-test-R:
(SEQ ID NO: 16)
TCCAGCAGCTGCTCTCTCTG.

The PCR amplification products of different strains were subjected to Sanger sequencing, and the sequencing results were compared with the wild-type CDPK9 gene. The CDPK9 genotypes were identified according to the following principles.

If there is a sequence with a double peak characteristic from the target site sequence, the genotype of the strain is a heterozygous genotype (the CDPK9 gene on one of the two homologous chromosomes is mutated, and the CDPK9 gene on the other chromosome is not mutated), and the strain is a T₀ generation transgenic tomato heterozygous mutant strain.

If there is a double peak sequence starting from the target site sequence and the CDPK9 gene in both homologous chromosomes are mutated, then the strain is a T₀ generation transgenic tomato biallelic mutation strain.

If the sequence with a specific single-peak characteristic starting from the target site sequence is the same as the CDPK9 gene sequence of the wild-type tomato, the genotype of the strain is wild type, that is, the CDPK9 gene sequence has not mutated; if it is different from the sequence of the CDPK9 gene of the wild-type tomato, the genotype of the strain is a homozygous genotype (both the CDPK9 genes on the two homologous chromosomes are mutated), and the strain is a T₀ generation transgenic tomato homozygous mutant strain.

In this case, a homozygous mutant strain of the CDPK9 gene of the T₀ generation was identified (as shown in FIG. 3A and FIG. 3B), which was used to identify the tomato SSC and sugar content phenotypes described below.

FIG. 3B shows the cdpk9-1 genotype of the homozygous mutant strain of the tomato CDPK9 gene; compared with the wild type (WT), the mutant strain has a 5-base deletion mutation, and the CDPK9 protein translation will undergo a frame-shift mutation, so cdpk9-1 is a loss-of-function mutant of the CDPK9 gene.

### Example 3: Determination of SSC, Sugar and Acid Content in Fruits of CDPK8 and CDPK9 Gene Knockout Tomato Mutants

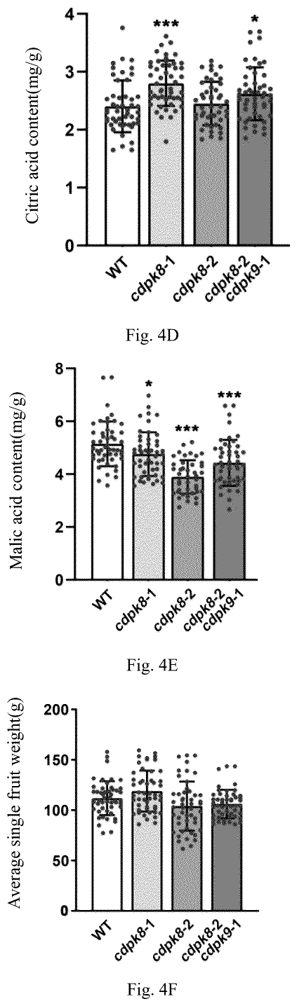

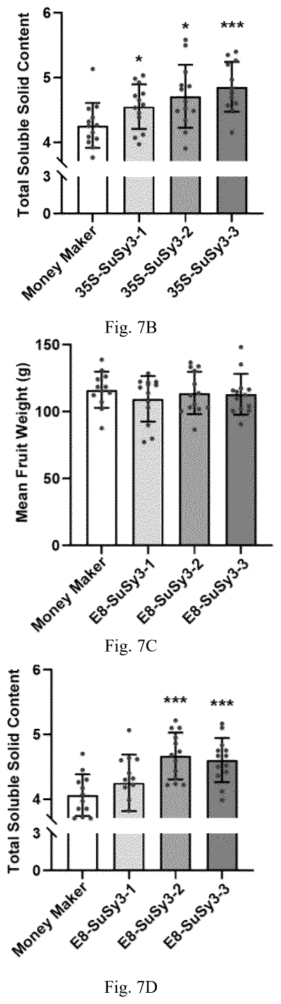

The T₂ generation CDPK8 homozygous mutant strains cdpk8-1 and cdpk8-2, and the CDPK8 and CDPK9 homozygous double gene mutant strains cdpk8-2/cdpk9-1 obtained in Examples 1 and 2 were planted in the greenhouse of the Shouguang Experimental Base of the Institute of Vegetables and Flowers, Chinese Academy of Agricultural Sciences in the autumn of 2021. The detection results of SSC, sugar and acid content and average single fruit weight of wild-type plants and mutants are shown in FIGS. 4A-4F. Particularly, in FIGS. 4A-4F and 7A-7D: * indicates P-Value <0.05; ** indicates P-Value <0.01; *** indicates P-Value <0.001.

Through systematic identification of the SSC, sugar and acid contents of the mutants, it was found that: the fruit SSC, glucose, fructose and citric acid contents of the homozygous mutant of CDPK8 and the homozygous double gene mutant strains of CDPK8 and CDPK9 are increased, the malic acid content is decreased, and the single fruit weight does not change significantly, indicating that the CDPK8 and CDPK9 genes play a key role in regulating the contents of SSC, glucose, fructose, citric acid and malic acid in tomato fruits.

### Example 4: CDPK Protein can Phosphorylate SuSy3 Protein and Promote its Degradation

In vitro phosphorylation experiments and protein degradation experiments demonstrated that, CDPK protein can phosphorylate tomato sucrose synthase SuSy3 and promote its degradation. The specific steps are as follows.

### Step 1. In Vitro Expression of SuSy3 and CDPK8 Proteins

The SuSy3-HIS and CDPK8-HIS expression vectors were constructed, and the SuSy3-HIS and CDPK8-HIS proteins were induced to express in vitro and were purified.

SEQ ID NO: 17 (SuSy3-CDS):
ATGGCTCAACGTGTTCTAACTCGTGTTCACAGTCTTCGTGAACGTCTTGA
TGCTACTTTGGATGCTCATCGCAATGAAATTTTGCTCTTTCTTTCAAGGA
TCGAAAGCCACGGGAAAGGGATCTTGAAACCTCACCAGCTACTGGCTGAG
TTTGAATCAATTCAGAAAGAAGACAAAGACAAACTGAATGATCATGCCTT
TGAAGAAGTCCTGAAATCCACTCAGGAAGCAATTGTTTTGCCCCCATGGG
TTGCACTTGCTATTCGTTTGAGGCCCGGTGTGTGGGAATATGTCCGTGTG
AATGTTAATGCTCTTAGTGTTGAGGAGCTGACTGTGCCTGAGTTTTTGCA
ATTCAAGGAAGAACTTGTTAACGGAACTTCCAGTGATAACTTTGTTCTTG
AATTGGATTTTGAGCCCTTCACTGCATCATTTCCAAAACCAACCCTCACG
AAATCAATTGGAAATGGAGTTGAATTCCTCAACAGGCACCTCTCTGCTAA
AATGTTCCATGACAAGGAAAGCATGACCCCTCTTCTCGAGTTTCTTCGAG
TTCACCACTACAATGGAAAGTCAATGATGCTGAATGATAGAATTCAGAAT
TTGTATACTCTCCAAAAAGTCCTGAGGAAGGCCGAGGAATACCTCACCAC
CCTTTCGCCAGAAACTTCATACTCCTCATTTGAGCACAAGTTCCAAGAAA
TTGGCTTGGAGAGAGGTTGGGGTGACACCGCAGAGCGTGTTCTAGAGATG
ATCTGCATGCTCCTGGATCTCCTTGAGGCTCCTGACTCATGTACTCTTGA
GAAGTTCCTTAGTAGAATTCCTATGGTTTTCAATGTAGTTATACTTTCAC
CTCATGGATATTTCGCCCAGGAAAATGTCTTGGGTTACCCCGACACTGGT
GGTCAGGTTGTCTATATTTTGGATCAAGTTCCTGCCTTGGAGCGTGAGAT
GCTCAAGCGCATAAAGGAGCAAGGACTTGATATCAAACCGCGTATTCTTA
TTGTTACTCGGCTTCTCCCTGATGCAGTTGGTACCACTTGTGGTCAGCGA
CTCGAGAAGGTATTTGGAACTGAGCATTCACATATTCTTAGGGTCCCCTT
TAGGACTGAAAAGGGCATTGTTCGCAAATGGATCTCTCGTTTTGAAGTCT
GGCCATACATGGAGACTTTCATTGAGGATGTGGGGAAAGAAATAACCGCA
GAACTGCAAGCTAAGCCAGATCTTATTATTGGAAACTATAGTGAGGGAAA
CCTTGCAGCCTCCTTGTTGGCTCACAAGTTAGGTGTAACACAGTGCACCA
TTGCTCATGCATTGGAGAAAACCAAATATCCTGATTCTGACATTTACTTG
AACAAATTTGACGAGAAATACCACTTCTCAGCTCAGTTCACAGCTGATCT
TATAGCAATGAATCATACTGATTTCATTATCACCAGCACCTTCCAGGAGA
TAGCAGGAAGCAAGGACACTGTTGGACAGTATGAGAGCCACATGGCCTTC
ACAATGCCTGGATTGTATAGAGTTGTTCATGGCATTGATGTGTTCGACCC
CAAATTCAACATTGTGTCACCAGGAGCTGATGTGAATCTCTATTTCCCAT
ACTCCGAAAAGGAAAAGAGATTGACAACTTTTCACCCTGAAATTGAAGAC
TTGCTGTTTAGCGATGTTGAGAACGAAGAACACCTGTGTGTGTTGAAGGA
CAGGAATAAGCCCATCATATTCACCATGGCAAGATTGGACCGAGTGAAGA
ACTTAACTGGACTTGTCGAGTGGTATGCTAAGAATCCACGACTAAGGGAG
TTGGTTAACCTTGTAGTGGTTGGTGGAGACCGAAGAAAGGAATCCAAAGA
CTTGGAAGAGCAGGCAGAGATGAAGAAGATGTATGAACTTATAAAGACTC
ACAATTTGAATGGCCAGTTCCGATGGATTTCTTCCCAGATGAACCGCGTG
AGGAATGGGGAACTCTACAGGTACATTGCTGACACAAGGGGAGCTTTCGT
GCAGCCTGCATTCTACGAGGCTTTCGGTCTGACTGTTGTTGAGGCCATGA
GCTGCGGTTTGCCTACATTTGCAACTAATCAAGGTGGTCCAGCTGAGATC
ATCGTTCATGGAAAGTCTGGTTTCCAAATTGATCCATACCATGGCGAGCA
GGCTGCTGATCTCCTCGCTGAGTTCTTCGAGAAATGTAAGGTAGACCCTT
CACATTGGGAAGCCATTTCCAAGGGTGGCCTTAAGCGTATACAGGAGAAG
TACACATGGCAAATCTACTCCGACCGGCTGTTGACACTAGCTGCTGTTTA
CGGGTTCTGGAAGCACGTTTCCAAGCTTGATCGTCTTGAAATTCGTCGTT
ATCTTGAGATGTTTTACGCTCTCAAATTCCGCAAGCTGGCTGAACTTGTC
CCATTGGCTGTTGAGTAA.
SEQ ID NO: 18 (SuSy3-protein):
MAQRVLTRVHSLRERLDATLDAHRNEILLFLSRIESHGKGILKPHQLLAE
FESIQKEDKDKLNDHAFEEVLKSTQEAIVLPPWVALAIRLRPGVWEYVRV
NVNALSVEELTVPEFLQFKEELVNGTSSDNFVLELDFEPFTASFPKPTLT
KSIGNGVEFLNRHLSAKMFHDKESMTPLLEFLRVHHYNGKSMMLNDRIQN
LYTLQKVLRKAEEYLTTLSPETSYSSFEHKFQEIGLERGWGDTAERVLEM
ICMLLDLLEAPDSCTLEKFLSRIPMVFNVVILSPHGYFAQENVLGYPDTG
GQVVYILDQVPALEREMLKRIKEQGLDIKPRILIVTRLLPDAVGTTCGQR
LEKVFGTEHSHILRVPFRTEKGIVRKWISRFEVWPYMETFIEDVGKEITA
ELQAKPDLIIGNYSEGNLAASLLAHKLGVTQCTIAHALEKTKYPDSDIYL
NKFDEKYHFSAQFTADLIAMNHTDFIITSTFQEIAGSKDTVGQYESHMAF
TMPGLYRVVHGIDVFDPKFNIVSPGADVNLYFPYSEKEKRLTTFHPEIED
LLFSDVENEEHLCVLKDRNKPIIFTMARLDRVKNLTGLVEWYAKNPRLRE
LVNLVVVGGDRRKESKDLEEQAEMKKMYELIKTHNLNGQFRWISSQMNRV
RNGELYRYIADTRGAFVQPAFYEAFGLTVVEAMSCGLPTFATNQGGPAEI
IVHGKSGFQIDPYHGEQAADLLAEFFEKCKVDPSHWEAISKGGLKRIQEK
YTWQIYSDRLLTLAAVYGFWKHVSKLDRLEIRRYLEMFYALKFRKLAELV
PLAVE.
SEQ ID NO: 19 (SuSy3-genome):
ATGGCTCAACGTGTTCTAACTCGTGTTCACAGTCTTCGTGAACGTCTTGA
TGCTACTTTGGATGCTCATCGCAATGAAATTTTGCTCTTTCTTTCAAGGT
ATGGTCTTACAGCCATGTTTTCGCTTTTTGTAATTTTCGTTTGGTATGTT
TCGCAAGCCTAAATAGTTTTGGGGATACAAACTTAGTACTGCCTTATTCC
TTTGTTTGGTTATTGATCTCGGAATAAGTTATATCAGAATTAGTAATTAG
TACCTCGATAAGTTATCTCTGATGGAATAATAGTACTGTTAACTAGTAAT
CCTGGAACAATACAATGAAAATGACAAAAATCCCCCAGAGGTCCCTTAGA
TCCTTTTTTACTTAATAAGGTGGAAGGTATTCTCGTAAACAAATAACTTT
CGTTCTTAAAAATTATGCAATGAATGTTTGTCCTAGTAAAACTTGTCTTC
AAACCAAACGACCCCTAATTGACGGACAAATATATGGTTCCATCAAGTAC
ATCATGAGTTATTCTTGCTGTTTTTGGCTGTATCAGGATCGAAAGCCACG
GGAAAGGGATCTTGAAACCTCACCAGCTACTGGCTGAGTTTGAATCAATT
CAGAAAGAAGACAAAGACAAACTGAATGATCATGCCTTTGAAGAAGTCCT
GAAATCCACTCAGGTAACTTGGTTTTGTTGTTAGGTTGATTATATTTAAT
TTTCTTACTAAGCTAACATGTGTATCTGTTTGTACTTGCATTTGAAATTT
TCTTTTGCGTTGTTCGTCATACTAGGAAGCAATTGTTTTGCCCCCATGGG
TTGCACTTGCTATTCGTTTGAGGCCCGGTGTGTGGGAATATGTCCGTGTG
AATGTTAATGCTCTTAGTGTTGAGGAGCTGACTGTGCCTGAGTTTTTGCA
ATTCAAGGAAGAACTTGTTAACGGAACGTAAGTTTTACGTTTGAATTTGA
TGATGAGTTATCTAATCAACATGTTCTTAGAATCTTTTTGATTAATGTTG
TGATTTTCCCTGATGCAGTTCCAGTGATAACTTTGTTCTTGAATTGGATT
TTGAGCCCTTCACTGCATCATTTCCAAAACCAACCCTCACGAAATCAATT
GGAAATGGAGTTGAATTCCTCAACAGGCACCTCTCTGCTAAAATGTTCCA
TGACAAGGAAAGCATGACCCCTCTTCTCGAGTTTCTTCGAGTTCACCACT
ACAATGGAAAGGTAAATTTAGTGTTATGGTGGTTTTTCTCCGATAAATTT
CGAGTATGGAGTTACTGATTTTTGCATCCAACAAATGATTAACATGTTCG
AACAATCATTTTCTGTGCAGTCAATGATGCTGAATGATAGAATTCAGAAT
TTGTATACTCTCCAAAAAGTCCTGAGGAAGGCCGAGGAATACCTCACCAC
CCTTTCGCCAGAAACTTCATACTCCTCATTTGAGCACAAGTTCCAAGAAA
TTGGCTTGGAGAGAGGTTGGGGTGACACCGCAGAGCGTGTTCTAGAGATG
ATCTGCATGCTCCTGGATCTCCTTGAGGCTCCTGACTCATGTACTCTTGA
GAAGTTCCTTAGTAGAATTCCTATGGTTTTCAATGTAGTTATACTTTCAC
CTCATGGATATTTCGCCCAGGAAAATGTCTTGGGTTACCCCGACACTGGT
GGTCAGGTGCATTGCTTATTTGTGATCACTTTGTCTTACTCCTTAGAAAC
CTATTGGGGCAAGTGCTGAGTTCCCGTTCCTTTAATTTGCAGGTTGTCTA
TATTTTGGATCAAGTTCCTGCCTTGGAGCGTGAGATGCTCAAGCGCATAA
AGGAGCAAGGACTTGATATCAAACCGCGTATTCTTATTGTTAGTATTCTC
AGAGATTGTGCTTATAGATTATGATTATGCAGGATTTTGATTTGTTCTAA
TGCAACATCTGATCTTTGTTTAAATTCTCAGGTTACTCGGCTTCTCCCTG
ATGCAGTTGGTACCACTTGTGGTCAGCGACTCGAGAAGGTATTTGGAACT
GAGCATTCACATATTCTTAGGGTCCCCTTTAGGACTGAAAAGGGCATTGT
TCGCAAATGGATCTCTCGTTTTGAAGTCTGGCCATACATGGAGACTTTCA
TTGAGGTGAAGCAACTTTTCTGTATTCATTTTTCAGTCTTCTAGTTGATT
TTTGCAGCAATTTTCTACTTACACTAAAACTGTGACTTTAATACATTAGG
ATGTGGGGAAAGAAATAACCGCAGAACTGCAAGCTAAGCCAGATCTTATT
ATTGGAAACTATAGTGAGGGAAACCTTGCAGCCTCCTTGTTGGCTCACAA
GTTAGGTGTAACACAGGTCTGTAACATTGGTCACATGTATAAGATTGACT
TTGCATTTCCTTTCATTTGGAACTCGGAGTTATTAAGAATTCTCTTTTGC
GTTGATCTGCAGTGCACCATTGCTCATGCATTGGAGAAAACCAAATATCC
TGATTCTGACATTTACTTGAACAAATTTGACGAGAAATACCACTTCTCAG
CTCAGTTCACAGCTGATCTTATAGCAATGAATCATACTGATTTCATTATC
ACCAGCACCTTCCAGGAGATAGCAGGAAGGTATGACATCAATTCGCTCTA
TGATTAAATTGTTCTTCTTCCTGTTTCATTGTGTTTGATCCTAAAACATT
TTTCAATTTTCTTTAGCAAGGACACTGTTGGACAGTATGAGAGCCACATG
GCCTTCACAATGCCTGGATTGTATAGAGTTGTTCATGGCATTGATGTGTT
CGACCCCAAATTCAACATTGTGTCACCAGGAGCTGATGTGAATCTCTATT
TCCCATACTCCGAAAAGGAAAAGAGATTGACAACTTTTCACCCTGAAATT
GAAGACTTGCTGTTTAGCGATGTTGAGAACGAAGAACACCTGTATGTTTC
TATAATAGTGCAACATTGTGTTTACTGATATAAACAAGCTTGTACTGAAA
TAGATTTGTGTTCCTATATCAGGTGTGTGTTGAAGGACAGGAATAAGCCC
ATCATATTCACCATGGCAAGATTGGACCGAGTGAAGAACTTAACTGGACT
TGTCGAGTGGTATGCTAAGAATCCACGACTAAGGGAGTTGGTTAACCTTG
TAGTGGTTGGTGGAGACCGAAGAAAGGAATCCAAAGACTTGGAAGAGCAG
GCAGAGATGAAGAAGATGTATGAACTTATAAAGACTCACAATTTGAATGG
CCAGTTCCGATGGATTTCTTCCCAGATGAACCGCGTGAGGAATGGGGAAC
TCTACAGGTACATTGCTGACACAAGGGGAGCTTTCGTGCAGCCTGCATTC
TACGAGGCTTTCGGTCTGACTGTTGTTGAGGCCATGAGCTGCGGTTTGCC
TACATTTGCAACTAATCAAGGTGGTCCAGCTGAGATCATCGTTCATGGAA
AGTCTGGTTTCCAAATTGATCCATACCATGGCGAGCAGGCTGCTGATCTC
CTCGCTGAGTTCTTCGAGAAATGTAAGGTAGACCCTTCACATTGGGAAGC
CATTTCCAAGGGTGGCCTTAAGCGTATACAGGAGAAGTAAGCTGCTCATC
TTTTCGTCCTATCACGTGATCACTATTGAGTGCATTAATTCAAAGTGATT
CTAATCTCCTGTTGCTGTAGGTACACATGGCAAATCTACTCCGACCGGCT
GTTGACACTAGCTGCTGTTTACGGGTTCTGGAAGCACGTTTCCAAGCTTG
ATCGTCTTGAAATTCGTCGTTATCTTGAGATGTTTTACGCTCTCAAATTC
CGCAAGCTGGTATGTCTCTCTGCTTTCTGCACTTGTCCAATAGTCTAAAG
CAACTGTTAATGGAGCTTCTGTATTTTGTTTTGATTCAGGCTGAACTTGT
CCCATTGGCTGTTGAGTAA.

### Step 2: In Vitro Phosphorylation Experiments Prove that CDPK8 can Phosphorylate SuSy3 Protein

The in vitro expressed CDPK8-HIS protein or mCDPK8-His protein without kinase activity was incubated with SuSy3-HIS protein in kinase reaction buffer (50 mM Tris-HCl, pH 7.5, 10 mM MgCl₂, 1 mM DTT, 5 μCi γ-32P-ATP) at room temperature for 30 minutes. Subsequently, 5× SDS loading buffer (250 mM Tris-HCl, pH 6.8, 10% [w/v] SDS, 0.5% bromophenyl blue, 50 mM DTT) was added to terminate the reaction, and the samples were separated by 10% SDS-PAGE gel. After electrophoresis, Coomassie Brilliant Blue R250 was used for staining as a protein loading control, and phosphorylated proteins were observed by autoradiography. In vitro phosphorylation experiments demonstrated that: the CDPK8 protein itself has kinase activity and can phosphorylate the SuSy3 protein, while the mCDPK8 protein lacking two key amino acids has no kinase activity and cannot phosphorylate the SuSy3 protein (FIG. 5A). The products of co-incubation of SuSy3-HIS and CDPK8-HIS proteins, and the products of incubation of SuSy3-HIS protein alone were used as negative controls. Mass spectrometry analysis showed that: 14 amino acid residues on the SuSy3 protein can be phosphorylated by CDPK8 protein, and the sites are T7, S11, T19, S152, S165, S174, T176, S191, S219, S360, T430, S535, T543, and S706 in sequence.

### Step 3: In Vitro Protein Degradation Experiments Proved that Phosphorylation of SuSy3 by CDPK Promotes its Degradation

The total protein of wild-type tomato and cdpk8-1 mutant fruits at the red-ripe stage was extracted by using degradation buffer (300 mM Tris-HCl pH 8.0, 600 mM NaCl, 4 mM MgCl₂, 20 mM DTT) respectively, and then equal amounts of SuSy3-His recombinant protein and 5 mM ATP were added to incubate at room temperature (25° C.) for 0 h, 1 h, 2 h and 4 h. Then 5×SDS loading buffer was added to stop the reaction, and the changes in the content of SuSy3-His protein were detected by immunoblotting using His antibody. It was found that the protein content of SuSy3 protein in the extract of wild-type fruits is gradually decreased with the passage of time; while in the mutant cdpk8-1, the rate of protein degradation is significantly slowed down, indicating that phosphorylation of SuSy3 by CDPK8 promotes its protein degradation (FIG. 5B).

### Step 4: In Vitro Phosphorylation Experiments Proved that CDPK8 can Phosphorylate Serine 11 of SuSy3 Protein

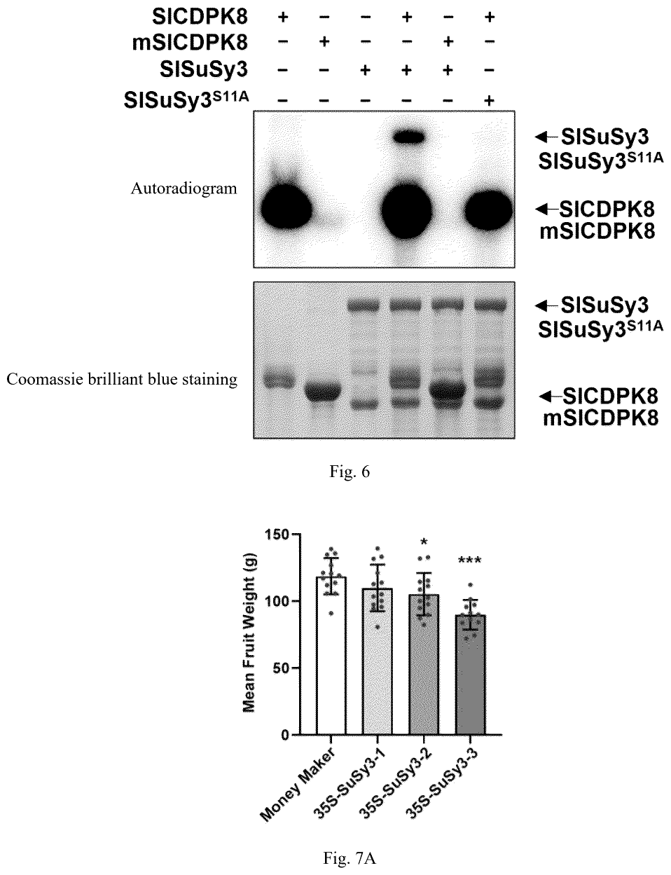

FIG. 6 shows an in vitro phosphorylation assay proving that CDPK8 can phosphorylate the serine at position 11 (S11) of the SuSy3 protein. When the serine at position 11 mutates to alanine (SISuSy3^{S11A}), there is no autoradiographic band, indicating that CDPK8 cannot phosphorylate SISuSy3^{S11A}, S11 is the modification site for CDPK8 to phosphorylate SuSy3.

### Example 5: Specific Overexpression of SuSy3 Gene in Fruits by Transgenic Technology

Agrobacterium-mediated transgenic technology is used to specifically over-express the SuSy3 gene in fruits, thereby obtaining transgenic plants in which the SuSy3 gene is specifically over-expressed in fruits. The specific steps are as follows.

### Step 1: Cloning SuSy3 Genomic Sequence

Primers were designed on the genome of SuSy3 gene, and the SuSy3 genomic sequence was cloned.

### Step 2: Construction of E8-SuSy3 Overexpression Vector

The original vector contains the E8 promoter sequence, which is a tomato fruit-specific expression promoter. The SuSy3 genomic sequence cloned in step 1 was further inserted into the vector to obtain the E8-SuSy3 overexpression vector.

### Step 3: Obtaining Transgenic Plants

The E8-SuSy3 overexpression vector obtained in step 2 was transferred to Agrobacterium competent cells EHA105 by heat shock transformation (Agrobacterium EHA105 competent cells were purchased from Shanghai Weidi Biotechnology Co., Ltd., and are available to the public) to obtain the recombinant bacteria EHA105/E8-SuSy3.

The recombinant bacteria EHA105/E8-SuSy3 was then transformed by Agrobacterium-infected tomato transformation method (the recombinant Agrobacterium was propagated at 28° C., and the propagated bacterial solution was used to infect tomatoes). After kanamycin resistance screening, the T₀ generation transgenic tomato plants were obtained.

### Step 4: Identification of Transgenic Plants Overexpressing SuSy3 Gene

The leaves of the T₀ generation transgenic tomato plants obtained in step 3 of Example 5 were collected, and genomic DNA was extracted as a template. PCR amplification was performed by using the following primer pairs, then detecting the PCR amplification products of different strains.

The sequences of the primers for detecting overexpression of the SuSy3 gene are as follows.

E8-test-F:
(SEQ ID NO: 20)
ATGGCATCCTCATATTGAGAT.
SuSy3-OE-R:
(SEQ ID NO: 21)
TATTCCTCGGCCTTCCTCAG.

Using the leaves of non-transgenic tomato plants as the control, the PCR amplification products of different strains were analyzed by agarose gel electrophoresis. The control leaves should have no bands, while some TO generation transgenic tomato plant leaves had bands. The strains with bands were the TO generation SuSy3 gene overexpression transgenic strains.

The SuSy3 gene overexpression transgenic line TO identified in this case was used for the identification of tomato SSC and fruit weight phenotypes as described below.

### Example 6: Phenotypic Identification of SuSy3 Overexpression Transgenic Lines and Fruit-Specifically Expressing Transgenic Lines

The same method as in Example 5 was used to prepare a SuSy3 overexpression transgenic lines (35S-SuSy3-1/2/3).

The SuSy3 overexpression transgenic lines (35S-SuSy3-1/2/3) and the fruit-specific expression transgenic lines (E8-SuSy3-1/2/3) obtained in Example 5 were subjected to SSC and fruit weight phenotype identification.

FIGS. 7A-7D shows the results of phenotypic identification of SuSy3 overexpression transgenic lines (35S-SuSy3-1/2/3) and fruit-specific expression transgenic lines (E8-SuSy3-1/2/3): overexpression of SuSy3 can significantly increase the SSC (FIG. 7B), and the SSC is inversely proportional to the fruit weight (FIG. 7A); specific expression of SuSy3 in fruits can significantly increase the SSC (FIG. 7D) and has no effect on the fruit weight (FIG. 7C).

The above are only preferred embodiments of the present disclosure. It should be pointed out that for a person skilled in the art, several improvements and modifications can be made without departing from the principle of the present disclosure. These improvements and modifications should also be regarded as falling in the protection scope of the present disclosure.

What is claimed is:

1. A plant or part thereof, wherein CDPK8 gene and/or CDPK9 gene of the plant or part thereof are knocked out;
wherein a polynucleotide of CDPK8 gene is at least one of the following nucleotide sequences:
(1) a sequence having at least 90% sequence identity to any one of the sequences set forth in SEQ ID NOs: 3 and 23-36; and
(2) a sequence encoding a protein comprising a sequence having at least 90% sequence identity and the same function as any one of the sequences set forth in SEQ ID NOs: 2 and 66-94; and
a polynucleotide of CDPK9 gene is at least one of the following nucleotide sequences:
(1) a sequence having at least 90% sequence identity to any one of the sequences set forth in SEQ ID NOs: 11 and 95-108; and
(2) a sequence encoding a protein comprising a sequence having at least 90% sequence identity and the same function as any one of the sequences set forth in SEQ ID NOs: 10 and 153-196.

2. The plant or part thereof of claim 1, wherein the polynucleotide of CDPK8 gene is at least one of the following nucleotide sequences:
(1) a sequence having at least 95% sequence identity to any one of the sequences set forth in SEQ ID NOs: 3 and 23-36; and
(2) a sequence encoding a protein comprising a sequence having at least 95% sequence identity and the same function as any one of the sequences set forth in SEQ ID NOs: 2 and 66-94; and/or
the polynucleotide of the CDPK9 gene is at least one of the following nucleotide sequences:
(1) a sequence having at least 95% sequence identity to any one of the sequences set forth in SEQ ID NOs: 11 and 95-108; and
(2) a sequence encoding a protein comprising a sequence having at least 95% sequence identity and the same function as any one of the sequences set forth in SEQ ID NOs: 10 and 153-196.

3. The plant or part thereof of claim 1, wherein the polynucleotide of CDPK8 gene is at least one of the following nucleotide sequences:
(1) a sequence having at least 98% sequence identity to any one of the sequences set forth in SEQ ID NOs: 3 and 23-36; and
(2) a sequence encoding a protein comprising a sequence having at least 98% sequence identity and the same function as any one of the sequences set forth in SEQ ID NOs: 2 and 66-94; and/or
the polynucleotide of the CDPK9 gene is at least one of the following nucleotide sequences:
(1) a sequence having at least 98% sequence identity to any one of the sequences set forth in SEQ ID NOs: 11 and 95-108; and
(2) a sequence encoding a protein comprising a sequence having at least 98% sequence identity and the same function as any one of the sequences set forth in SEQ ID NOs: 10 and 153-196.

4. The plant or part thereof of claim 1, wherein the polynucleotide of CDPK8 gene is at least one of the following nucleotide sequences:
(1) any one of SEQ ID NOs: 3 and 23-36; and
(2) a sequence encoding any one of SEQ ID NOs: 2 and 66-94; and/or
the polynucleotide of the CDPK9 gene is at least one of the following nucleotide sequences:
(1) any one of SEQ ID NOs: 11 and 95-108; and
(2) a sequence encoding any one of SEQ ID NOs: 10 and 153-196.

5. The plant or part thereof of claim 1, wherein the part thereof is protoplast, plant cell, tissue culture, fruit, pollen, seed, leaf, or flower.

6. A method for improving a quality of a plant fruit, wherein a phosphorylation level of SuSy3 protein is reduced by at least one of the following methods:
S1. inhibiting an expression of CDPK or inactivating a phosphorylase of CDPK; a polynucleotide of CDPK gene is a polynucleotide of CDPK8 gene and/or CDPK9 gene, a polynucleotide of CDPK8 gene is at least one of the following nucleotide sequences:
(1) a sequence having at least 90% sequence identity to any one of the sequences set forth in SEQ ID NOs: 3 and 23-36; and
(2) a sequence encoding a protein comprising a sequence having at least 90% sequence identity and the same function as any one of the sequences set forth in SEQ ID NOs: 2 and 66-94; and
a polynucleotide of the CDPK9 gene is at least one of the following nucleotide sequences:
(1) a sequence having at least 90% sequence identity to any one of the sequences set forth in SEQ ID NOs: 11 and 95-108; and
(2) a sequence encoding a protein comprising a sequence having at least 90% sequence identity and the same function as any one of the sequences set forth in SEQ ID NOs: 10 and 153-196; or
an amino acid sequence of CDPK protein is at least one of the following sequences:
(21) a sequence as set forth in any one of SEQ ID NOs: 2 and 66-94;
(22) a fusion protein obtained by connecting a tag to N-terminus and/or C-terminus of any of the sequences set forth in SEQ ID NOs: 2 and 66-94;
(23) a protein having the same function obtained by substituting and/or deleting and/or adding one or more amino acid residues in any of the sequences set forth in SEQ ID NOs: 2 and 66-94;
(24) a protein having at least 90% identity to any one of the sequences set forth in SEQ ID NOs: 2 and 66-94 and having the same function;
(25) a sequence as set forth in any one of SEQ ID NOs: 10 and 153-196;
(26) a fusion protein obtained by connecting a tag to the N-terminus and/or C-terminus of any of the sequences set forth in SEQ ID NOs: 10, and 153-196;
(27) a protein having the same function obtained by substituting and/or deleting and/or adding one or more amino acid residues in any of the sequences set forth in SEQ ID NOs: 10 and 153-196; and
(28) a protein having at least 90% identity with the sequence set forth in any one of SEQ ID NOs: 10 and 153-196 and having the same function;
S2. mutating one or more amino acids in SuSy3 protein, wherein a mutation site is a phosphorylation site mediated by CDPK, without altering the glycosyltransferase activity of the SuSy3 protein; or
S3. specifically overexpressing the SuSy3 protein in the fruit.

7. The method of claim 6, wherein the polynucleotide of CDPK8 gene is at least one of the following nucleotide sequences:
(1) a sequence having at least 95% sequence identity to any one of the sequences set forth in SEQ ID NOs: 3 and 23-36;
(2) a sequence encoding a protein comprising a sequence having at least 95% sequence identity and the same function as any one of the sequences set forth in SEQ ID NOs: 2 and 66-94; and/or
the polynucleotide of the CDPK9 gene is at least one of the following nucleotide sequences:
(1) a sequence having at least 95% sequence identity to any one of the sequences set forth in SEQ ID NOs: 11 and 95-108;
(2) a sequence encoding a protein comprising a sequence having at least 95% sequence identity and the same function as any one of the sequences set forth in SEQ ID NOs: 10 and 153-196; and/or
the amino acid sequence of CDPK protein is at least one of the following sequences:
(21) any one of SEQ ID NOs: 2 and 66-94;
(22) a fusion protein obtained by connecting a tag to N-terminus and/or C-terminus of any of the sequences as set forth in SEQ ID NOs: 2 and 66-94;
(23) a protein having the same function obtained by substituting and/or deleting and/or adding one or more amino acid residues in any of the sequences as set forth in SEQ ID NOs: 2 and 66-94;
(24) a protein having at least 95% identity to any one of the sequences set forth in SEQ ID NOs: 2 and 66-94 and having the same function;
(25) any one of SEQ ID NOs: 10 and 153-196;
(26) a fusion protein obtained by connecting a tag to the N-terminus and/or C-terminus of any of the sequences as set forth in SEQ ID NOs: 10, and 153-196;
(27) a protein having the same function obtained by substituting and/or deleting and/or adding one or more amino acid residues in any of the sequences as set forth in SEQ ID NOs: 10 and 153-196; and
(28) a protein having at least 95% identity with any one of the sequences set forth in SEQ ID NOs: 10 and 153-196 and having the same function.

8. The method of claim 6, wherein the polynucleotide of CDPK8 gene is at least one of the following nucleotide sequences:
(1) a sequence having at least 98% sequence identity to any one of the sequences set forth in SEQ ID NOs: 3 and 23-36;
(2) a sequence encoding a protein comprising a sequence having at least 98% sequence identity and the same function as any one of the sequences set forth in SEQ ID NOs: 2 and 66-94; and/or
the polynucleotide of the CDPK9 gene is at least one of the following nucleotide sequences:
(1) a sequence having at least 98% sequence identity to any one of the sequences set forth in SEQ ID NOs: 11 and 95-108;
(2) a sequence encoding a protein comprising a sequence having at least 98% sequence identity and the same function as any one of the sequences set forth in SEQ ID NOs: 10 and 153-196; and/or
the amino acid sequence of CDPK protein is at least one of the following sequences:
(21) any one of SEQ ID NOs: 2 and 66-94;
(22) a fusion protein obtained by connecting a tag to N-terminus and/or C-terminus of any of the sequences as set forth in SEQ ID NOs: 2 and 66-94;
(23) a protein having the same function obtained by substituting and/or deleting and/or adding one or more amino acid residues in any of the sequences as set forth in SEQ ID NOs: 2 and 66-94;
(24) a protein having at least 98% identity to any one of the sequences set forth in SEQ ID NOs: 2 and 66-94 and having the same function;
(25) any one of SEQ ID NOs: 10 and 153-196;
(26) a fusion protein obtained by connecting a tag to the N-terminus and/or C-terminus of any of the sequences as set forth in SEQ ID NOs: 10, and 153-196;
(27) a protein having the same function obtained by substituting and/or deleting and/or adding one or more amino acid residues in any of the sequences as set forth in SEQ ID NOs: 10 and 153-196; and
(28) a protein having at least 98% identity with any one of the sequences set forth in SEQ ID NOs: 10 and 153-196 and having the same function.

9. The method of claim 6, wherein the polynucleotide of CDPK8 gene is at least one of the following nucleotide sequences:
(1) any one of SEQ ID NOs: 3 and 23-36;
(2) a sequence encoding any one of SEQ ID NOs: 2 and 66-94; and/or
the polynucleotide of the CDPK9 gene is at least one of the following nucleotide sequences:
(21) any one of SEQ ID NOs: 2 and 66-94;
(2) a sequence encoding any one of SEQ ID NOs: 10 and 153-196; and/or
the amino acid sequence of CDPK protein is at least one of the following sequences:
(21) any one of SEQ ID NOs: 2 and 66-94;
(25) any one of SEQ ID NOs: 10 and 153-196.

10. The method of claim 6, wherein the plant is a berry plant.

11. The method of claim 6, wherein an improvement of the quality of plant fruits comprises: increasing the content of soluble solids, sugar, and citric acid in the plant fruits, reducing the content of malic acid, and maintaining the weight of the fruits; and the sugar comprises one or more of glucose, fructose and sucrose.

12. The method of claim 6, wherein in the above method S1, the method for inhibiting the expression of CDPK or inactivating the phosphorylase of CDPK comprises regulating a promoter and/or changing a coding region.

13. The method of claim 6, wherein in the above method S2, the mutation site is serine at position 11 of the SuSy3 protein.

14. The method of claim 6, wherein in the above method S3, the promoter for specifically overexpressing the SuSy3 protein in the fruit is E8 promoter.

15. An improved tomato plant or seed, comprising one or more genetic modifications and is capable of producing tomato fruits at maturity, and it comprises:
(i) at least one of the following: an average total soluble solids content which is at least 5% higher than that of a control tomato plant or seed, an average glucose level which is at least 5% higher than that of a control tomato plant or seed, and an average fructose level which is at least 5% higher than that of a control tomato plant or seed; and
(ii) an average fresh fruit weight substantially similar to that of a control tomato plant and seed;
wherein both the control tomato plant or seed and the modified tomato plant or seed comprise substantially the same genetic background, except for the one or more genetic modifications, and the one or more genetic modifications are located in a gene selected from the group consisting of: CDPK8, CDPK9, SuSy3, and a homolog thereof.

16. The improved tomato plant or seed of claim 15, wherein the one or more genetic modifications comprise at least one of the following: a CDPK8 deletion mutation, a CDPK9 deletion mutation, a double deletion mutation of CDPK8 and CDPK9, and an insertion of a sequence as set forth in SEQ ID NO: 22 in the CDPK8 promoter present in Hap2.

17. A commercial plant product, wherein it is a commercial plant product made from the plant or part thereof of claim 1; the commercial plant product comprises food, medicine, cosmetics/skin care product, feed or other products.

18. A method for producing a commercial plant product, comprising obtaining the plant or part thereof of claim 1 to produce the commercial plant product.
